SEQUENCE LISTING (41 sequences)

SEQ ID NO: 1 - protein, 17 aa, Artificial Sequence
  16B5 CDRL1
KSSQSLLNSRTRKNYLA

SEQ ID NO: 2 - protein, 7 aa, Artificial Sequence
  16B5 CDRL2
WASTRES

SEQ ID NO: 3 - protein, 8 aa, Artificial Sequence
  16B5 CDRL3
KQSYNLWT

SEQ ID NO: 4 - protein, 10 aa, Artificial Sequence
  16B5 CDRH1-V1
GFNIKDIYMH

SEQ ID NO: 5 - protein, 17 aa, Artificial Sequence
  16B5 CDRH2-V1
RIDPAYGNTKYDPKFQG

SEQ ID NO: 6 - protein, 8 aa, Artificial Sequence
  16B5 CDRH3-V1
RYDTAMDY

SEQ ID NO: 7 - protein, 112 aa, Artificial Sequence
  Murine 16B5 VL
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLWTFGGGTKLEFK

SEQ ID NO: 8 - protein, 117 aa, Artificial Sequence
  Murine 16B5 VH
EVQLQQSGAELVKPGASVRLSCTTSGFNIKDIYMHWVKQRPEQGLEWIGRIDPAYGNTKYDPKFQGKATITADTSSNTAYLQLSSLTSEDTAVYYCARRYDTAMDYWGQGTSVTVSS

SEQ ID NO: 9 - protein, 112 aa, Artificial Sequence
  Humanized 16B5 VL
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLWTFGQGTKLEIK

SEQ ID NO: 10 - protein, 117 aa, Artificial Sequence
  Humanized 16B5 VH
QVQLVQSGAEVKKPGATVKISCKVSGFNIKDIYMHWVQQAPGKGLEWMGRIDPAYGNTKYDPKFQGRVTITADTSTDTAYMELSSLRSEDTAVYYCARRYDTAMDYWGQGTLVTVSS

SEQ ID NO: 11 - protein, 217 aa, Artificial Sequence
  Humanized 16B5 light chain
DIVMTQSPDSLAVSLGERATINCKSSQSLLNSRTRKNYLAWYQQKPGQPPKLLIYWASTRESGVPDRFSGSGSGTDFTLTISSLQAEDVAVYYCKQSYNLWTFGQGTKLEIKVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 443 aa, Artificial Sequence
  Humanized 16B5 heavy chain
QVQLVQSGAEVKKPGATVKISCKVSGFNIKDIYMHWVQQAPGKGLEWMGRIDPAYGNTKYDPKFQGRVTITADTSTDTAYMELSSLRSEDTAVYYCARRYDTAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - DNA, 720 bp, Artificial Sequence
  Humanized 16B5 light chain nucleic acid
atggtgctgcagacccaggtgttcatctccctgctgctgtggatctccggcgcctacggcgacatcgtgatgacccagtcccccgactccctggccgtgtccctgggcgagagagccaccatcaactgcaagtcctcccagtccctgctgaactcccggacccggaagaactacctggcctggtatcagcagaagcctggccagcctcctaagctgctgatctactgggcctccacccgggagtccggcgtgcctgaccggttctccggctccggcagcggcaccgacttcaccctgaccatcagctccctgcaggccgaggacgtggccgtgtactactgcaagcagtcctacaacctgtggaccttcggccagggcaccaagctggagatcaagcggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 14 - DNA, 1389 bp, Artificial Sequence
  Humanized 16B5 heavy chain nucleic acid
atggactggacctggcggatcctgttcctggtggccgctgctaccggcacccacgcccaggtgcagctggtgcagtctggcgccgaggtgaagaagcctggcgccaccgtcaagatcagctgcaaggtgtccggcttcaacatcaaggacatctacatgcactgggtgcagcaggctccaggcaagggactggagtggatgggccggatcgaccctgcctacggcaacaccaagtacgaccctaagttccagggccgggtgaccatcaccgccgacacctccaccgacaccgcctacatggaactgtcctccctgcggtccgaggacaccgccgtgtactactgcgcccggagatacgacaccgccatggattactggggccagggcaccctggtgaccgtgtcctccgcttccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 15 - protein, 17 aa, Artificial Sequence
  21B12 CDRL1
KSSQSLLYSSNQKNYLA

SEQ ID NO: 16 - protein, 7 aa, Artificial Sequence
  21B12 CDRL2
WASTRES

SEQ ID NO: 17 - protein, 9 aa, Artificial Sequence
  21B12 CDRL3
QQYYIYPRT

SEQ ID NO: 18 - protein, 10 aa, Artificial Sequence
  21B12 CDRH1-v1
GYTFTNYGMH

SEQ ID NO: 19 - protein, 17 aa, Artificial Sequence
  21B12 CDRH2-v1
WINTYTGEPTYADDFKG

SEQ ID NO: 20 - protein, 9 aa, Artificial Sequence
  21B12 CDRH3-v1
DGFLYFFDY

SEQ ID NO: 21 - protein, 113 aa, Artificial Sequence
  Murine 21B12 VL
DIVMSQSPSSLAVSVGEKVTMSCKSSQSLLYSSNQKNYLAWYQQRPGQSPKLLIYWASTRESGVPDRFTGSGSGTDFTLTISSVKAEDLAVYYCQQYYIYPRTFGGGTKLEIK

SEQ ID NO: 22 - protein, 118 aa, Artificial Sequence
  Murine 21B12 VH
QIQLVQSGPELKKPGETVKISCKASGYTFTNYGMHWVKQAPGKGLKWMGWINTYTGEPTYADDFKGRFAFSLETSASTAYLQINNLKNEDTATYFCARDGFLYFFDYWGQGTTLTVSS

SEQ ID NO: 23 - protein, 113 aa, Artificial Sequence
  Humanized 21B12 VL
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYIYPRTFGQGTKLEIK

SEQ ID NO: 24 - protein, 118 aa, Artificial Sequence
  Humanized 21B12 VH
QVQLVQSGSELKKPGASVKVSCKASGYTFTNYGMHWVRQAPGQGLEWMGWINTYTGEPTYADDFKGRFVFSLDTSVSTAYLQISSLKAEDTAVYYCARDGFLYFFDYWGQGTLVTVSS

SEQ ID NO: 25 - protein, 220 aa, Artificial Sequence
  Humanized 21B12 light chain
DIVMTQSPDSLAVSLGERATINCKSSQSLLYSSNQKNYLAWYQQKPGQPPKLLIYWASTRESGVPDRFSGSGSGTDFTLTISSLQAEDVAVYYCQQYYIYPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 444 aa, Artificial Sequence
  Humanized 21B12 heavy chain
QVQLVQSGSELKKPGASVKVSCKASGYTFTNYGMHWVRQAPGQGLEWMGWINTYTGEPTYADDFKGRFVFSLDTSVSTAYLQISSLKAEDTAVYYCARDGFLYFFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 27 - DNA, 723 bp, Artificial Sequence
  Humanized 21B12 light chain nucleic acid
atggtgctgcagacccaggtgttcatctccctgctgctgtggatctctggcgcctacggcgacatcgtgatgacccagtcccccgactctctggctgtgtccctgggcgagcgggccaccatcaactgcaagtcctcccagtccctgctgtactcctccaaccagaagaactacctggcctggtatcagcagaagcctggccagcctcctaagctgctgatctactgggcctccacccgggaatctggcgtgcctgaccggttctccggctctggctccggcaccgacttcaccctgaccatcagctccctgcaggccgaggacgtggccgtgtactactgccagcagtactacatctaccctcggaccttcggccagggcaccaagctggaaatcaagcggaccgtggccgctccttccgtgttcatcttccccccttccgacgagcagctgaagtccggcaccgcctctgtggtgtgcctgctgaacaacttctacccccgggaggccaaggtgcagtggaaggtggacaacgccctgcagtccggcaactcccaggaatccgtcaccgagcaggactccaaggactctacctactccctgtcctccaccctgaccctgtccaaggccgactacgagaagcacaaggtgtacgcctgcgaagtgacccaccagggcctgtcctctcccgtgaccaagtccttcaaccggggcgagtgctga

SEQ ID NO: 28 - DNA, 1392 bp, Artificial Sequence
  Humanized 21B12 heavy chain nucleic acid
atggactggacctggcggatcctgtttctggtggccgctgctaccggcacacacgcccaggtgcagctggtgcagtccggctccgagctgaagaaacctggcgcctccgtgaaggtgtcctgcaaggcctccggctacaccttcaccaactacggcatgcactgggtgcgccaggcacctggacagggactggaatggatgggctggatcaacacctacaccggcgagcctacctacgccgacgacttcaagggcagattcgtgttctccctggacacctccgtgtccaccgcctacctgcagatctcctccctgaaggccgaggacaccgccgtgtactactgcgccagggacggcttcctgtacttcttcgactactggggccagggcaccctggtgaccgtgtcctctgcctccaccaagggcccttccgtgttccctctggccccttgctcccggtccacctctgagtctaccgccgctctgggctgcctggtgaaggactacttccctgagcctgtgacagtgtcctggaactctggcgccctgacctctggcgtgcacaccttccctgccgtgctgcagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgtcctgctcctcctgtggctggccctagcgtgttcctgttccctcctaagcctaaggacaccctgatgatctcccggacccctgaagtgacctgcgtggtggtggacgtgtcccacgaggaccctgaggtgcagttcaattggtacgtggacggcgtggaggtgcacaacgccaagaccaagcctcgggaggaacagttcaactccaccttccgggtggtgtccgtgctgaccgtggtgcaccaggactggctgaacggcaaagaatacaagtgcaaggtgtccaacaagggcctgcctgcccctatcgaaaagaccatctctaagaccaagggccagcctcgcgagcctcaggtgtacaccctgcctccctcccgcgaggaaatgaccaagaaccaggtgtccctgacctgtctggtgaagggcttctacccttccgatatcgccgtggagtgggagtctaacggccagcctgagaacaactacaagaccacccctcctatgctggactccgacggctccttcttcctgtacagcaagctgacagtggacaagtcccggtggcagcagggcaacgtgttctcctgctccgtgatgcacgaggccctgcacaaccactacacccagaagtccctgtccctgtctcctggcaagtga

SEQ ID NO: 29 - protein, 111 aa, Artificial Sequence
  20E11 variable light chain
DIVLTLSPASLAVSLGQRATISCRASQSVNSSNYSYMHWYQQKPGQPPKLLIKYASNLESGVPARFSGSGSGTHFTLNIHPVEEEDTATYYCQHSWEIPWTFGGGTKLEIK

SEQ ID NO: 30 - protein, 118 aa, Artificial Sequence
  20E11 variable heavy chain
QIQLVQSGPELKKPGETVKISCKASGYTFTDYSMHWVKQAPGKGLKWMGWINTETGEPTYADDFKGRFAFSLETSASTAYLQINNLKNEDTATYFCARTGSSGYFDCWGQGTTLTVSS

SEQ ID NO: 31 - protein, 106 aa, Artificial Sequence
  11E2 variable light chain Gr1
ENVLTQSPAIMSASPGEKVTMTCSASSSVSYMHWYQQKSSTSPKLWIYDTSKLASGVPGRFSGSGSGNSYSLTISSMEAEDVATYYCFQGSGYPFTFGSGTKLEIK

SEQ ID NO: 32 - protein, 107 aa, Artificial Sequence
  11E2 variable light chain Gr2
DIQMTQSPSSLSASLGGKVTITCKASQDINKYIAWYQHKPGKGPRLLIHYTSTLQPGIPSRFSGSGSGRDYSFSISNLEPEDIATYYCLQYDNLLRTFGGGTKLEIK

SEQ ID NO: 33 - protein, 120 aa, Artificial Sequence
  11E2 variable heavy chain
EVQLQQSGPELEKPGASVKISCKASGYSFTGYNMNWVKQNNGKSLEWIGNIDPYYGTPNYNQKFKGKATLTVDKSSSTAYMQLKSLTSEDSAVYYCALNSLLRLNAMDYWGQGTSVTVSS

SEQ ID NO: 34 - protein, 118 aa, Artificial Sequence
  16C11 variable heavy chain
EVQLQQSGPELGKPGASVKISCKASGYSFTGYNMYWVKQSHRKSLEWIGYIDPYNGDTSYNQKSKGKATLTADRSSSTAYMHLNSLTSEDSGIYYCARGAYGSSYAYWGQGTLVAVSA

SEQ ID NO: 35 - protein, 8 aa, Artificial Sequence
  16B5 CDRH1-v2
GFNIKDIY

SEQ ID NO: 36 - protein, 8 aa, Artificial Sequence
  16B5 CDRH2-v2
IDPAYGNT

SEQ ID NO: 37 - protein, 10 aa, Artificial Sequence
  16B5 CDRH3-v2
ARRYDTAMDY

SEQ ID NO: 38 - protein, 8 aa, Artificial Sequence
  21B12 CDRH1-v2
GYTFTNYG

SEQ ID NO: 39 - protein, 8 aa, Artificial Sequence
  21B12 CDRH2-v2
INTYTGEP

SEQ ID NO: 40 - protein, 9 aa, Artificial Sequence
  21B12 CDRH3-v2
DGFLYFFDY

SEQ ID NO: 41 - protein, 75 aa, Artificial Sequence
  amino acids 421-443 of C-terminal portion of beta subunit of human clusterin
LTQGEDQYYLRVTTVASHTSDSDVPSGVTEVVVKLFDSDPITVTVPVEVSRKNPKFMETVAEKALQEYRKKHREE

(19) United States
(12) Patent Application Publication
(10) Pub. No.: US 2024/0317884 A1
(43) Pub. Date: Sep. 26, 2024

(54) METHOD FOR ALLOWING IMMUNE CELLS INFILTRATION IN TUMORS

(71) Applicant: Alethia Biotherapeutics Inc., Montréal (CA)

(72) Inventor: Mario FILION, Longueuil (CA)

(73) Assignee: Alethia Biotherapeutics Inc., Montréal, QC (CA)

(21) Appl. No.: 18/287,331

(22) PCT Filed: Apr. 26, 2022
(86) PCT No.: PCT/CA2022/050632

§ 371 Date: Oct. 18, 2023

(30) Foreign Application Priority Data
Apr. 27, 2021 (WO) PCT/CA2021/050572

(51) Int. Cl. C07K 16/30; A61K 31/337; A61P 35/04; A61K 39/00

(52) U.S. Cl. CPC C07K 16/30; A61K 31/337; A61P 35/04; A61K 2039/505; A61K 2039/545; C07K 2317/24; C07K 2317/565; C07K 2317/90

(57) ABSTRACT

The present disclosure generally relates to a method for treating a subject having cancer. The method of the present disclosure allows infiltration of immune cells into the tumor microenvironment and may be used to modulate an antitumor immune response. The method of the present disclosure is based on the administration of an anti-cancer therapy comprising an anti-clusterin antibody or antigen binding fragment thereof. The anti-cancer therapy of the present disclosure may be used as an adjuvant or neoadjuvant therapy. Combination therapy, medicament and kits for such use are also provided.

### TECHNICAL FIELD

The present disclosure generally relates to a method for treating a subject having cancer. The method of the present disclosure allows infiltration of immune cells into the tumor microenvironment and may be used to modulate an antitumor immune response. The method of the present disclosure is based on the administration of an anti-cancer therapy comprising an anti-clusterin antibody or antigen binding fragment thereof. The anti-cancer therapy of the present disclosure may be used as an adjuvant or neoadjuvant therapy. Combination therapy, medicament and kits for such use are also provided.

### BACKGROUND

The molecular mechanisms responsible for the occurrence of metastatic carcinomas are beginning to be elucidated with the identification of key regulators. Increasing evidence points to tumor cell epithelial-to-mesenchymal (EMT) as an important contributing process to metastatic evolution. The occurrence of EMT during tumor progression permits epithelial tumor cells, that are non-invasive and non-metastatic, to move from the primary tumor, invade the surrounding tissue, enter the bloodstream and finally disseminate to, and proliferate at secondary sites. In addition, epithelial cancer cells that undergo EMT adopt a behavior that is very similar to cancer stem cells (CSCs) including an inherent resistance to chemotherapy and immune evasion (Shibue et al., 2017; Terry et al. 2017).

Despite the short-term effectiveness of first- and second-line therapies such as chemotherapeutic agents and immune checkpoint inhibitors in cancer therapy, a high proportion of subjects become refractory to these therapies due to the resistance of tumor cells to anti-cancer agents and the survival of tumor-initiating cells, two events that ultimately result in an increase in metastasis and poor subject survival.

The mechanism underlying resistance to immune checkpoint inhibitors can vary. However, it is generally accepted that checkpoint inhibitors work best against so-called immunologically hot tumors that is tumors that have been invaded by T cells creating an inflamed tumor. In contrast, immunologically cold tumors are poorly responsive to immunotherapy because these tumors haven't been recognized or haven't provoke a strong immune response and therefore T cells have not penetrated to tumor or its microenvironment.

Patients who have received a prior first line immune checkpoint inhibitor as a single agent are offered platinum-based chemotherapy in second line. Single agent docetaxel can be administered as second- or third-line therapy following failure of immune checkpoint inhibition and platinum doublet chemotherapy administered simultaneously or consecutively. Since most patients eventually progress following immunochemotherapy and since docetaxel has very limited efficacy in this setting, novel therapies are urgently needed.

### SUMMARY

The Applicant came to the unexpected discovery that treatment with an anti-clusterin antibody or antigen binding fragment thereof such as AB-16B5 leads to increased intra-tumor immune infiltration (see international patent application No. PCT/CA2021/050572 filed on Apr. 27, 2021, the entire content of which is incorporated herein by reference).

Preliminary data of a phase II clinical trial reported herein show similar findings. This phase II clinical trial is evaluating a combination treatment comprising an anti-clusterin antibody (AB-16B5, a.k.a., humanized 16B5) and docetaxel in subjects with metastatic non-small cell lung cancer who previously experienced disease progression after receiving therapy with an anti-programmed death 1 (PD-1) or PD-L1 immune checkpoint antibody and a platinum-containing doublet treatment, administered simultaneously or sequentially (NCT04364620).

The anti-cancer therapy of the present disclosure may thus be used to modulate the antitumor immune response. Anti-clusterin antibodies or antigen binding fragments thereof may thus potentiate the therapeutic effect of other anti-cancer agents or may help overcoming resistance.

The present disclosure therefore relates in some aspects and embodiments, to an anti-cancer therapy that comprises an anti-clusterin antibody or antigen binding fragment thereof.

The present disclosure also relates to a method of treating a subject having cancer comprising administering the anti-cancer therapy of the present disclosure to the subject.

The present disclosure also relates to a method of modulating an antitumor immune response by administering the anti-cancer therapy of the present disclosure to a subject need.

For example, the present disclosure encompasses treating a subject having cancer by a method that comprises modulating an antitumor immune response by administering an anti-cancer therapy as disclosed herein to a subject as disclosed herein.

In another example, the present disclosure encompasses treating a subject having cancer by a method that comprises promoting infiltration of immune cells in the tumor microenvironment by administering an anti-cancer therapy as disclosed herein to a subject as disclosed herein.

In some embodiments, the subject may have one or more lesions that are characterized as immunologically cold.

In other embodiments, the subject may have one or more lesions showing signs of an epithelial to mesenchymal transition (EMT) signature.

In accordance with the present disclosure, the anti-cancer therapy consists of an anti-clusterin antibody or antigen binding fragment thereof provided as a single anti-cancer agent.

In accordance with the present disclosure, the anti-cancer therapy comprises an anti-clusterin antibody or antigen binding fragment thereof and another anti-cancer agent. Accordingly, the anti-cancer therapy may be a combination therapy.

In some embodiments, the combination therapy comprises the anti-clusterin antibody or antigen binding fragment thereof and radiation therapy.

In other embodiments, the combination therapy comprises the anti-clusterin antibody or antigen binding fragment thereof and chemotherapy.

Exemplary embodiments of chemotherapy include an alkylating agent, an anti-metabolite, an alkaloid, an anti-tumor antibiotic or combination thereof.

In some instances, the alkylating agent may be selected, for example, from altretamine, busulfan, carboplatin, carmustine, cisplatin, cyclophosphamide, dacarbazine, ifosfamide, lomustine, melphalan, temozolomide, trabectedin or derivatives or analogs thereof.

In some instances, the anti-metabolite may be selected, for example, 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, trifluridine, tipiracil or derivatives or analogs thereof.

In some instances, the alkaloid may be selected, for example, from vincristine, vinblastine, vinorelbine, taxanes, etoposide, teniposide, irinotecan, topotecan or derivatives or analogs thereof.

Exemplary embodiments of taxane includes docetaxel, paclitaxel and derivatives or analogues including for example and without limitations, Abraxane®, Cabazitaxel, larotaxel, milataxel, ortataxel, tesetaxel and others described in Ojima et al., Expert Opin Ther Pat. 2016: 26(1): 1-20, the entire content of which is incorporated herein by reference.

In some instance, the taxane is docetaxel.

In some instance, the taxane is paclitaxel.

In some instances, the anti-tumor antibiotic may be selected, for example, from daunorubicin, doxorubicin, doxorubicin liposomal, epirubicin, idarubicin, valrubicin, derivatives or analogs thereof.

The present disclosure therefore provides a method for allowing infiltration of immune cells in a tumor (e.g., a solid tumor) microenvironment, which comprises a step of administering to a subject in need thereof an anti-clusterin antibody or an antigen binding fragment thereof.

In accordance with the present disclosure anti-clusterin antibody or an antigen binding fragment thereof may be used in a subject in need for allowing infiltration of immune cells in a tumor (e.g., solid tumor) microenvironment or in the manufacture of a medicament for allowing infiltration of immune cells in a tumor (e.g., solid tumor) microenvironment.

In some embodiments, the method of the present disclosure encompasses administering the anti-cancer therapy disclosed herein as a neo-adjuvant therapy.

In some embodiments, the method of the present disclosure encompasses administering the anti-cancer therapy disclosed herein as an adjuvant therapy.

In some embodiments, the method of the present disclosure encompasses administering the anti-cancer therapy prior to tumor resection or surgery.

In some embodiments, the method of the present disclosure encompasses administering the anti-cancer therapy after tumor resection or surgery.

In some embodiments, the method of the present disclosure encompasses administering the anti-cancer therapy both prior to and after tumor resection or surgery.

The term “tumor” as used herein refers to a primary tumor or to a tumor lesion (lesion). Accordingly, in some instances, the method of the present disclosure is for allowing infiltration of immune cells in a primary tumor microenvironment. In other instances, the method of the present disclosure is for allowing infiltration of immune cells in the microenvironment of one or more lesions.

The present disclosure also provides a method for treating a subject having cancer (e.g., a solid tumor), which comprises a step of administering to a subject in need thereof an anti-clusterin antibody or an antigen binding fragment thereof.

In accordance with the present disclosure the anti-clusterin antibody or an antigen binding fragment thereof may be used for treating a subject having cancer or in the manufacture of a medicament for treating a subject having cancer.

In an exemplary embodiment, the anti-clusterin antibody or antigen binding fragment thereof is administered at a dose and/or an administration interval and/or for a treatment period sufficient to result in infiltration of immune cells in the tumor (e.g., solid tumor) microenvironment.

The method of the present disclosure may also comprise a step of administering chemotherapy to the subject in need.

In an exemplary embodiment, the chemotherapy is docetaxel and is administered at a dose and/or an administration interval and/or for a treatment period sufficient to allow chemotherapy-induced immunogenic modulation of tumor.

In an exemplary embodiment, both the anti-clusterin antibody or antigen binding fragment thereof and docetaxel are each administered at a dose and/or an administration interval and/or for a treatment period sufficient to allow infiltration of immune cells in a tumor microenvironment and/or chemotherapy-induced immunogenic modulation of tumor.

In accordance with the present disclosure, the subject in need is a subject having a tumor or having cancer and a functional immune system.

In accordance with the present disclosure, the subject in need is a subject having a tumor or having cancer and an adequate organ and immune function.

The present disclosure therefore provides a method of treating a subject having cancer, that comprises a step of administering a combination therapy comprising an anti-clusterin antibody or antigen binding fragment thereof and docetaxel, wherein the subject has a functional immune system or an adequate organ and immune function.

In accordance with the present disclosure, the anti-clusterin antibody or an antigen binding fragment thereof and docetaxel combination therapy may be used for treating a subject having cancer or in the manufacture of a medicament for treating a subject having cancer, wherein the subject has a functional immune system or an adequate organ and immune function.

In accordance with the present disclosure, the method may result in an increase (in the presence or in the amount) of immune cells in the tumor microenvironment.

In accordance with the present disclosure, the method may result in an increase in the activity of immune cells in the tumor microenvironment.

In accordance with the present disclosure, the method may result in modulation of an immune response towards tumor cells.

In accordance with the present disclosure, the anti-clusterin antibody or antigen binding fragment thereof or combination therapy may result in a less immune-refractory tumor microenvironment.

In accordance with the present disclosure, the anti-clusterin antibody or antigen binding fragment thereof and docetaxel combination therapy may contribute to the creation of a more favorable immune environment with an increased anti-tumor cytotoxic T cell activity.

In accordance with the present disclosure, the cell killing activity of specific CD8⁺ cytotoxic T cells may be enhanced after treatment with the combination therapy.

In accordance with the present disclosure, the method may result in the tumor being more susceptible to a chemotherapeutic usually associated with resistance mediated by MDR1/P-glycoprotein.

In accordance with the present disclosure, the method may result in the tumor being more susceptible to treatment by immunotherapy.

The method of the present disclosure may therefore also include a step of administering immunotherapy after treatment with the anti-cancer therapy disclosed herein.

In some embodiments, the immunotherapy comprises cellular immunotherapy (CAR-T, TILs, etc.).

In some embodiments, the immunotherapy comprises an immune checkpoint inhibitor. An exemplary embodiment of an immune checkpoint inhibitor is an immune checkpoint antibody.

In other aspects and embodiments, method of the present disclosure may result, for example, in regression of a lesion. In some instances, the regression may be complete. In other instances, the regression may be partial. For example, the treatment may result, for example, in a decrease in the size of a lesion.

In yet other aspects and embodiments, the treatment may result, for example, in stabilization in the growth of a lesion.

It is to be understood herein that a lesion is considered to decrease in size when measurements indicates that it is smaller than previous measurements or than baseline measurement.

It is also to be understood herein that an increase of less than approximately 20% in the size of a lesion, in comparison with previous measurements or baseline measurements is considered as a stabilization in the growth of a lesion.

In some embodiments, the method of the present disclosure may result in an increase of, or less than approximately 20% in the size of a lesion in comparison with baseline measurements. In other embodiments, the method of the present disclosure may result in an increase of, or less than approximately 15% in the size of a lesion in comparison with baseline measurements. In other embodiments, the method of the present disclosure may result in an increase of, or less than approximately 10% in the size of a lesion in comparison with baseline measurements. In yet other embodiments, the method of the present disclosure may result in an increase of, or less than approximately 5% in the size of a lesion in comparison with baseline measurements.

In some instances, the size of a lesion may increase or may remain stable while the number of tumor cells within the lesion decreases. For example, tumor cells might be replaced by fibrotic tissue and inflammatory cells and as such no decrease in the size of a lesion is detected. Accordingly, in some instances, the number of live tumor cells may decrease, and the number of immune cells may increase thereby resulting in pseudogrogression.

It is also to be understood herein that an increase in the size of a lesion conjugated with a decrease in the number of tumor cells within that lesion is also considered as a stabilization in the growth of a lesion.

It is also to be understood herein that a reduction of the metabolic activity of a lesion (with or without growth) is also considered as a stabilization in the growth of a lesion.

The size or metabolic activity of a lesion may be assessed by several methods known to a person skilled in the art, including for example and without limitations, by CT scan or positron emission tomography (PET).

In accordance with the present disclosure, the anti-clusterin antibody or antigen binding fragment thereof comprises a light chain variable region comprising the complementarity determining regions (CDRs) of the light chain variable region set forth in SEQ ID NO:9 and a heavy chain variable region comprising the CDRs of the heavy chain variable region set forth in SEQ ID NO:10.

In accordance with the present disclosure, the anti-clusterin antibody or antigen binding fragment thereof comprises a light chain variable region having an amino acid sequence having at least 80%, at least 85%, at least 90%, at least 95%, at least 99% identity with the amino acid sequence set forth in SEQ ID NO:9 or is identical to or comprises the amino acid sequence set forth in SEQ ID NO:9 and a heavy chain variable region having an amino acid sequence at least 80%, at least 85%, at least 90%, at least 95%, at least 99% identity with the amino acid sequence set forth in SEQ ID NO:10 or is identical to or comprises the amino acid sequence set forth in SEQ ID NO:10.

In accordance with the present disclosure, the anti-clusterin antibody or antigen binding fragment thereof comprises a light chain having an amino acid sequence having at least 80%, at least 85%, at least 90%, at least 95%, at least 99% identity with the amino acid sequence set forth in SEQ ID NO:11 or is identical to or comprises the amino acid sequence set forth in SEQ ID NO:11 and a heavy chain having an amino acid sequence having at least 80%, at least 85%, at least 90%, at least 95%, at least 99% identity with the amino acid sequence set forth in SEQ ID NO:12 or is identical to or comprises the amino acid sequence set forth in SEQ ID NO:12.

In accordance with the present disclosure, the antibody or antigen binding fragment thereof is capable of competing with an antibody comprising a light chain variable region having an amino acid sequence set forth in SEQ ID NO:9 and a heavy chain variable region having an amino acid sequence set forth in SEQ ID NO: 10 for the binding of clusterin (e.g., secreted clusterin (sCLU) or tumor-associated sCLU (TA-sCLU)) or for binding to a polypeptide comprising the amino acid sequence set forth in SEQ ID NO:41.

In accordance with the present disclosure, the anti-clusterin antibody or antigen binding fragment thereof comprises the amino acid sequence of CDRs, light chain and heavy chain variable regions or light chain and heavy chain set forth in Table 9.

In accordance with the present disclosure, the method may result in infiltration of immune cells in a primary tumor microenvironment.

In accordance with the present disclosure, the method may result in infiltration of plasmocytes in a tumor microenvironment.

In accordance with the present disclosure, the method may result in infiltration of T cells in a tumor microenvironment. In some embodiments, the T cells comprise CD4⁺ T cells. In some embodiments, the T cells comprise CD8⁺ T cells. In other embodiments, T cells comprises both CD4⁺ T cells and CD8⁺ T cells.

In accordance with the present disclosure, the method may result in infiltration of B cells in a tumor microenvironment.

In accordance with the present disclosure, the method may result in infiltration of T cells and B cells in a tumor microenvironment.

In accordance with the present disclosure, the method may result in necrosis of a tumor.

In some embodiments, the anti-clusterin antibody or antigen binding fragment thereof is administered once weekly.

In some embodiments, the anti-clusterin antibody or antigen binding fragment thereof is administered twice weekly.

In some embodiments, the anti-clusterin antibody or antigen binding fragment thereof is administered once every two weeks.

In some embodiments, the anti-clusterin antibody or antigen binding fragment thereof is administered once every three weeks.

In some embodiments, the anti-clusterin antibody or antigen binding fragment thereof is administered once every four weeks.

In some embodiments, docetaxel is administered once every week.

In some embodiments, docetaxel is administered once every two weeks.

In some embodiments, docetaxel is administered once every three weeks.

In some embodiments, docetaxel is administered once every four weeks.

In accordance with the present disclosure, the anti-clusterin antibody or antigen binding fragment thereof is administered at a dose of between approximately 3 mg/kg and approximately 20 mg/kg, such as for example, between approximately 4 mg/kg and approximately 20 mg/kg, between approximately 5 mg/kg and approximately 20 mg/kg, between approximately 6 mg/kg and approximately 20 mg/kg, between approximately 6, mg/kg and approximately 18 mg/kg, between approximately 6 mg/kg and approximately 17 mg/kg, between approximately 6 mg/kg and approximately 16 mg/kg, between approximately 6 mg/kg and approximately 15 mg/kg, between approximately 6 mg/kg and approximately 14 mg/kg, between approximately 6 mg/kg and approximately 13 mg/kg, between approximately 6 mg/kg and approximately 12 mg/kg, between approximately 7 mg/kg and approximately 18 mg/kg, between approximately 7 mg/kg and approximately 17 mg/kg, between approximately 7 mg/kg and approximately 16 mg/kg, between approximately 7 mg/kg and approximately 15 mg/kg, between approximately 7 mg/kg and approximately 14 mg/kg, between approximately 7 mg/kg and approximately 13 mg/kg, between approximately 7 mg/kg and approximately 12 mg/kg, between approximately 8 mg/kg and approximately 18 mg/kg, between approximately 8 mg/kg and approximately 17 mg/kg, between approximately 8 mg/kg and approximately 16 mg/kg, between approximately 8 mg/kg and approximately 15 mg/kg, between approximately 8 mg/kg and approximately 14 mg/kg, between approximately 8 mg/kg and approximately 13 mg/kg, between approximately 8 mg/kg and approximately 12 mg/kg, between approximately 9 mg/kg and approximately 18 mg/kg, between approximately 9 mg/kg and approximately 17 mg/kg, between approximately 9 mg/kg and approximately 16 mg/kg, between approximately 9 mg/kg and approximately 15 mg/kg, between approximately 9 mg/kg and approximately 14 mg/kg, between approximately 9) mg/kg and approximately 13 mg/kg, between approximately 9 mg/kg and approximately 12 mg/kg, between approximately 10 mg/kg and approximately 18 mg/kg, between approximately 10 mg/kg and approximately 17 mg/kg, between approximately 10 mg/kg and approximately 16 mg/kg, between approximately 10 mg/kg and approximately 15 mg/kg, between approximately 10 mg/kg and approximately 14 mg/kg, between approximately 10 mg/kg and approximately 13 mg/kg, or between approximately 10 mg/kg and approximately 12 mg/kg. In some embodiments, the anti-clusterin antibody or antigen binding fragment thereof is administered at a dose of approximately 6 mg/kg.

In some embodiments, the anti-clusterin antibody or antigen binding fragment thereof is administered at a dose of approximately 9 mg/kg.

In some embodiments, the anti-clusterin antibody or antigen binding fragment thereof is administered at a dose of approximately 12 mg/kg.

In accordance with the present disclosure docetaxel is administered at a dose of between approximately 60 mg/m² to approximately 100 mg/m² such as for example, between approximately 60 mg/m² to approximately 95 mg/m², between approximately 60 mg/m² to approximately 90 mg/m², between approximately 60 mg/m² to approximately 85 mg/m², between approximately 60 mg/m² to approximately 80 mg/m², between approximately 60 mg/m² to approximately 75 mg/m², between approximately 75 mg/m² to approximately 95 mg/m², between approximately 75 mg/m² to approximately 90 mg/m², between approximately 75 mg/m² to approximately 85 mg/m², between approximately 75 mg/m² to approximately 80 mg/m², between approximately 70 mg/m² to approximately 95 mg/m², between approximately 70 mg/m² to approximately 90 mg/m², between approximately 70 mg/m² to approximately 85 mg/m², between approximately 70 mg/m² to approximately 80 mg/m², or between approximately 70 mg/m² to approximately 75 mg/m².

In some embodiments, docetaxel is administered at a dose of approximately 60 mg/m².

In some embodiments, docetaxel is administered at a dose of approximately 75 mg/m².

In some embodiments, the subject is treated with the anti-clusterin antibody or antigen binding fragment thereof at a dose of approximately 12 mg/kg once weekly and docetaxel at a dose of approximately 75 mg/m² once every three weeks.

In some embodiments, the subject is treated with the anti-clusterin antibody or antigen binding fragment thereof at a dose of approximately 12 mg/kg once weekly and docetaxel at a dose of approximately 60 mg/m² once every three weeks.

In some embodiments, the subject is treated with the anti-clusterin antibody or antigen binding fragment thereof at a dose of approximately 9 mg/kg once weekly and docetaxel at a dose of approximately 75 mg/m² once every three weeks.

In some embodiments, the subject is treated with the anti-clusterin antibody or antigen binding fragment thereof at a dose of approximately 9 mg/kg once weekly and docetaxel at a dose of approximately 60 mg/m² once every three weeks.

In some embodiments, the subject is treated with the anti-clusterin antibody or antigen binding fragment thereof at a dose of approximately 6 mg/kg once weekly and docetaxel at a dose of approximately 75 mg/m² once every three weeks.

In some embodiments, the subject is treated with the anti-clusterin antibody or antigen binding fragment thereof at a dose of approximately 6 mg/kg once weekly and docetaxel at a dose of approximately 60 mg/m² once every three weeks.

In some embodiments, the subject is treated with the anti-clusterin antibody or antigen binding fragment thereof at a dose of approximately 3 mg/kg once weekly and docetaxel at a dose of approximately 75 mg/m² once every three weeks.

In some embodiments, the subject is treated with the anti-clusterin antibody or antigen binding fragment thereof at a dose of approximately 3 mg/kg once weekly and docetaxel at a dose of approximately 60 mg/m² once every three weeks.

In some embodiments, the anti-clusterin antibody or antigen binding fragment thereof and docetaxel may be administered on same day.

In some embodiments, the anti-clusterin antibody or antigen binding fragment thereof and docetaxel may be administered on same day and separately.

In some embodiments, the anti-clusterin antibody or antigen binding fragment thereof and/or docetaxel may be administered by infusion over approximately a 1-hour time frame.

In some embodiments, the anti-clusterin antibody or antigen binding fragment thereof and docetaxel are both administered over the entire course of the treatment period.

In some embodiments, the anti-clusterin antibody or antigen binding fragment thereof and docetaxel are both essentially administered over the entire course of the treatment period.

In some embodiments, the anti-clusterin antibody or antigen binding fragment thereof and docetaxel are both administered during the first cycles of the treatment period and then are both essentially administered during the remaining cycles of the treatment period.

In some embodiments, the anti-clusterin antibody or antigen binding fragment thereof and docetaxel are both administered during the first one, the first two, the first three, the first four or the first five cycles of the treatment period and then are both essentially administered during the remaining cycles of the treatment period.

In some embodiments, the anti-clusterin antibody or antigen binding fragment thereof and docetaxel are both administered at each cycle of treatment.

In some embodiments, the subject in need does not receive concurrent treatment other than the anti-clusterin antibody or antigen binding fragment thereof and/or docetaxel.

In some embodiments, the subject in need does not require concurrent treatment other than the anti-clusterin antibody or antigen binding fragment thereof and/or docetaxel.

In accordance with the present disclosure, the subject in need is a human in need.

In accordance with the present disclosure, the subject in need is a subject having a tumor characterized as metastatic.

In accordance with the present disclosure, the subject in need is a subject having a carcinoma.

In accordance with the present disclosure, the subject in need is a subject having a metastatic carcinoma.

In accordance with the present disclosure, the subject in need is a subject having or selected for having a tumor characterized as immunologically cold.

In some embodiments, the subject in need is a subject having or selected for having a tumor characterized as immunologically warm or hot that is non-responsive to immunotherapy.

In some embodiments the subject has or is selected for having a carcinoma that has failed prior anti-cancer treatment.

In other embodiments, the subject in need is a subject having or selected for having a carcinoma that progressed after a first line immune checkpoint therapy.

In some exemplary embodiments, the subject is a subject that has not received prior treatment comprising a PD-1 or PD-L1 immune checkpoint inhibitor.

In some exemplary embodiments, the subject is a subject that has received prior treatment comprising a PD-1 or PD-L1 immune checkpoint inhibitor.

In some exemplary embodiments, the subject is a subject that has not received prior treatment comprising an anti-PD-1 or anti-PD-L1 immune checkpoint antibody.

In other exemplary embodiments, the subject is a subject that has received or is selected for having received prior treatment comprising an anti-PD-1 or anti-PD-L1 immune checkpoint antibody.

In further embodiments, the subject has received or is selected for having received prior treatment comprising an anti-PD-1 or anti-PD-L1 immune checkpoint antibody and chemotherapy.

In yet further embodiments, the subject has received or is selected for having received prior treatment comprising an anti-PD-1 or anti-PD-L1 immune checkpoint antibody and a chemotherapeutic doublet treatment.

In other embodiments, the subject has or is selected for having a carcinoma that progressed after treatment comprising an anti-PD-1 or anti-PD-L1 immune checkpoint antibody.

In other embodiments, the subject has or is selected for having a carcinoma that progressed after chemotherapy.

In additional embodiments, the subject has or is selected for having a carcinoma that progressed after anti-PD-1 or anti-PD-L1 immune checkpoint antibody and chemotherapy doublet treatment.

In yet other embodiments, the subject has or is selected for having a carcinoma that has failed prior treatment with anti-PD-1 or anti-PD-L1 immune checkpoint antibody and a platinum-containing doublet treatment.

In some embodiments, the subject has or is selected for having a carcinoma that has failed prior treatment comprising an anti-PD-1 immune checkpoint antibody.

In some embodiments, the subject has or is selected for having a carcinoma that has failed prior treatment comprising an PD-L1 immune checkpoint antibody.

In some embodiments, the subject has or is selected for having a carcinoma that has failed prior treatment comprising CTLA-4 immune checkpoint antibody.

In some embodiments, the subject has or is selected for having a carcinoma that has failed prior treatment comprising a combination of CTLA-4 and PD-1 immune checkpoint antibody.

In some embodiments, the subject has or is selected for having a carcinoma that has failed prior treatment with an anti-PD-1 immune checkpoint antibody and a platinum-containing doublet treatment.

In some embodiments, the subject has or is selected for having a carcinoma that has failed prior treatment with an anti-PD-L1 immune checkpoint antibody and a platinum-containing doublet treatment.

In additional embodiments, the subject has or is selected for having a carcinoma that has failed prior treatment with an anti-PD-1 or PD-L1 immune checkpoint antibody and a platinum-containing doublet treatment.

In some embodiments, the subject has or is selected for having metastatic a carcinoma that has failed prior treatment with an anti-PD-1 or PD-L1 immune checkpoint antibody and a platinum-containing doublet treatment.

In some embodiment the doublet treatment may be provided simultaneously.

In some embodiment the doublet treatment may be provided sequentially.

In some embodiments, the PD-L1 tumor proportion score (TPS) is assessed prior to administration of the anti-cancer therapy of the present disclosure. For example, the PD-L1 TPS may be determined in one or more tumor lesions. In some embodiments, the anti-cancer therapy is administered to a subject having low expression of programmed death ligand 1 (PD-L1), no evidence of PD-L1 expression or that is not treatable with an anti-PD-1 or anti-PD-L1 immune checkpoint antibody.

In yet other embodiments, the subject in need is a subject having or selected for having a carcinoma that has failed prior treatment with an immune checkpoint therapy and a platinum-containing doublet treatment (e.g., simultaneously or sequentially).

In accordance with the present disclosure, the subject in need has a tumor that expresses or secrete clusterin.

In exemplary embodiments, the subject in need may have for example, endometrial cancer, breast cancer, liver cancer, prostate cancer, renal cancer, bladder cancer, cervical cancer, ovarian cancer, colorectal cancer, pancreatic cancer, lung cancer, gastric cancer, head and neck cancer, thyroid cancer, cholangiocarcinoma, mesothelioma, melanoma.

In accordance with the present disclosure, the subject in need is a subject having non-small cell lung cancer (NSCLC).

In some embodiments, the subject in need is a subject having metastatic NSCLC.

In some embodiments, the subject in need is a subject having stage III to IV NSCLC.

In accordance with the present disclosure, the subject in need is a subject having breast cancer.

In some embodiments, the subject in need is a subject having metastatic breast cancer.

In accordance with the present disclosure, the subject in need is a subject having prostate cancer.

In some embodiments, the subject in need is a subject having metastatic prostate cancer.

In accordance with the present disclosure, the subject in need is a subject having gastric cancer.

In some embodiments, the subject in need is a subject having metastatic gastric cancer.

In accordance with the present disclosure, the subject in need is a subject having head and neck cancer.

In some embodiments, the subject in need is a subject having metastatic head and neck cancer.

In accordance with the present disclosure, the subject in need is a subject having thyroid cancer.

In some embodiments, the subject in need is a subject having metastatic thyroid cancer.

In accordance with the present disclosure, the subject in need is a subject having ovarian cancer.

In some embodiments, the subject in need is a subject having metastatic ovarian cancer.

In accordance with the present disclosure, the subject in need is a subject having endometrial cancer.

In some embodiments, the subject in need is a subject having metastatic endometrial cancer.

In accordance with the present disclosure, the subject in need is a subject having liver cancer.

In some embodiments, the subject in need is a subject having metastatic liver cancer.

In accordance with the present disclosure, the subject in need is a subject having colorectal cancer.

In some embodiments, the subject in need is a subject having metastatic colorectal cancer.

In accordance with the present disclosure, the subject in need is a subject having pancreatic cancer.

In some embodiments, the subject in need is a subject having metastatic pancreatic cancer.

In accordance with the present disclosure, the subject in need is a subject having cholangiocarcinoma.

In some embodiments, the subject in need is a subject having metastatic cholangiocarcinoma.

In accordance with the present disclosure, the subject in need is a subject having mesothelioma.

In some embodiments, the subject in need is a subject having metastatic mesothelioma.

In accordance with the present disclosure, the subject in need is a subject having melanoma.

In some embodiments, the subject in need is a subject having metastatic melanoma.

In accordance with the present disclosure, the subject in need is a subject having bladder cancer.

In accordance with the present disclosure, the subject in need is a subject having metastatic bladder cancer.

In accordance with the present disclosure, the subject in need is a subject having cervical cancer.

In accordance with the present disclosure, the subject in need is a subject having metastatic cervical cancer.

In accordance with the present disclosure, the subject in need is a subject that is not immunosuppressed or has not received an immunosuppressive medication within 14, 13, 12, 11, 10, 9, 8, 7, 6, 5, 4, 3, 2 days or 1 day prior to treatment.

In accordance with the present disclosure, the subject in need is a subject that has not received prior treatment with docetaxel.

In accordance with the present disclosure the subject is treated for one or more cycles of treatment. In some embodiments, one cycle of treatment is approximately 21 days.

In an exemplary embodiment, the subject is treated for at least one cycle of treatment.

In another exemplary embodiment, the subject is treated for at least two cycles of treatment.

In an additional exemplary embodiment, the subject is treated for at least three cycles of treatment.

In yet an additional exemplary embodiment, the subject is treated for at least four cycles of treatment.

In other exemplary embodiments, the subject is treated or receives four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more treatment cycles.

In some embodiments, the treatment cycles are consecutive.

In some embodiments, the treatment cycles are interrupted for a period of time (ranging from one day to several weeks or months). In some embodiments, at least one treatment cycle is interrupted. In other embodiments, more than one treatment cycles are interrupted. In other embodiments, treatment is interrupted after a certain period of time determined by a physician or clinician.

In accordance with the present disclosure, the infiltration of immune cells in the tumor microenvironment is confirmed by biopsy.

In accordance with the present disclosure, the infiltration of immune cells in the tumor microenvironment is confirmed by imaging (e.g., magnetic resonance imaging).

In accordance with the present disclosure, the method comprises a step of administering immunotherapy (immune checkpoint inhibitors, cellular immunotherapy etc.) after one or more cycles of the anti-cancer therapy.

For example, the method comprises a step of administering immunotherapy (immune checkpoint inhibitors, cellular immunotherapy) after one or more cycles of anti-clusterin antibody or antigen binding fragment thereof as a single agent or in combination therapy with docetaxel.

In some embodiments, the immunotherapy comprises cellular immunotherapy (CAR-T, TILs, etc.).

In some embodiments, the immunotherapy comprises an immune checkpoint inhibitor.

In some embodiments, the method comprises a step of administering ipilimumab, nivolumab, pembrolizumab, cemiplimab, atezolizumab, avelumab, or durvalumab after one or more cycles of anti-clusterin antibody or antigen binding fragment thereof as a single agent or in combination therapy with docetaxel.

In some embodiments, the subject is treated with one or more cycles of the anti-cancer therapy and is subsequently treated with an immune checkpoint inhibitor that he has not previously received.

For example, the subjects is treated with one or more cycles of anti-clusterin antibody or antigen binding fragment thereof as a single agent or in combination therapy with docetaxel and is subsequently treated with an immune checkpoint inhibitor that he has not previously received.

The present disclosure also provides an anti-cancer therapy comprising an anti-clusterin antibody or antigen binding fragment thereof.

In some embodiments, the anti-cancer therapy is for use in a method as described herein.

In some embodiments, the anti-cancer therapy is for use in treating subjects as described herein.

For example, the anti-cancer therapy of the present disclosure may be used for treating a subject having an early-stage cancer.

In another example, the anti-cancer therapy of the present disclosure may be used for treating a subject having a late-stage cancer.

In some embodiments, the anti-cancer therapy may be used for allowing infiltration of immune cells in a tumor (e.g., a solid tumor) microenvironment in a subject having cancer.

The anti-cancer therapy may be used for example, in a method of treating a subject having cancer by promoting infiltration of immune cells in the tumor microenvironment.

In some embodiments, the anti-cancer therapy of the present disclosure may be used as a neoadjuvant therapy. For example, the anti-cancer therapy may be used prior to tumor resection.

The anti-cancer therapy of the present disclosure may be used as an adjuvant therapy. For example, the anti-cancer therapy of the present disclosure may be used after tumor resection.

In some embodiments, the anti-cancer therapy may be used prior to and after tumor resection.

Accordingly, the anti-cancer therapy may be used in a subject having a resectable tumor. In some embodiments, the anti-cancer therapy is a combination therapy as described herein.

In some embodiments, the anti-cancer therapy comprises an anti-clusterin antibody or antigen binding fragment thereof in combination with docetaxel for the treatment of a subject having cancer wherein the subject has a functional immune system or an adequate organ and immune function.

In some embodiments, the anti-clusterin antibody or antigen binding fragment thereof is formulated as an injectable solution at a concentration of approximately 10 mg/mL.

In some embodiments, the anti-clusterin antibody or antigen binding fragment thereof is formulated as an intravenous infusion for delivery of a dose of between approximately 3 mg/kg and approximately 20 mg/kg.

In some embodiments, docetaxel is formulated is formulated as an injectable solution at a concentration of between approximately 10 mg/mL to approximately 40 mg/mL.

In some embodiments, docetaxel is formulated as an intravenous infusion for delivery of a dose of between approximately 60 mg/m² to approximately 100 mg/m².

The present disclosure also provides a combination therapy comprising a pharmaceutical composition comprising an anti-clusterin antibody or antigen binding fragment thereof formulated for administration at a dose of between approximately 3 mg/kg and approximately 20 mg/kg and a pharmaceutical composition comprising docetaxel formulated for administration at a dose of between approximately 60 mg/m² to 100 mg/m².

In accordance with the present disclosure, the anti-clusterin antibody or antigen binding fragment thereof comprises CDRs, variable regions or light chain and heavy chains as described herein.

In accordance with the present disclosure, the combination therapy is used or is for use in treating a subject in need.

In accordance with the present disclosure, the combination therapy is used or is for use in treating a subject having cancer as described herein.

In some exemplary embodiments, the combination therapy is used or is for used in treating a subject having a carcinoma.

In some exemplary embodiments, the combination therapy is used or is for use in treating a subject having a metastatic carcinoma.

In accordance with the present disclosure, the combination therapy is used or is for used in treating a subject having or selected for having a carcinoma that progressed after a first line immune checkpoint therapy.

In accordance with the present disclosure, the combination therapy is used or is for use in treating a subject having or selected for having a carcinoma that has failed prior treatment with a platinum-containing doublet treatment and immune checkpoint therapy (e.g., simultaneously or sequentially).

In accordance with the present disclosure, the combination therapy is used or is for use in treating a subject having or selected for having a carcinoma that has failed prior treatment with a platinum-containing doublet treatment and an anti-PD1 or PD-L1 immune checkpoint antibody (e.g., simultaneously or sequentially).

In some exemplary embodiments, the combination therapy is used or is for use in treating a subject having non-small cell lung cancer. In some embodiments, the subject has metastatic NSCLC or stage III to IV NSCLC.

In some exemplary embodiments, the combination therapy is used or is for use in treating a subject having breast cancer, prostate cancer, gastric cancer, head and neck cancer, thyroid cancer or ovarian cancer.

In other exemplary embodiments, the combination therapy is used or is for use in treating a subject having metastatic breast cancer, metastatic prostate cancer, metastatic gastric cancer, metastatic head and neck cancer, metastatic thyroid cancer or metastatic ovarian cancer.

In some exemplary embodiments, the combination therapy is used or is for use in a subject that is not immunosuppressed or that has not received an immunosuppressive medication within 7 days prior to treatment.

In some exemplary embodiments, the combination therapy is used or is for use in a subject that has not received prior treatment with docetaxel.

In some embodiments, the pharmaceutical composition comprising the anti-clusterin antibody or antigen binding fragment thereof and the pharmaceutical composition comprising docetaxel are both administered over the entire course of the treatment period.

In accordance with the present disclosure, the anti-clusterin antibody or antigen binding fragment thereof is used or is for use at a dosage disclosed herein.

In accordance with the present disclosure, docetaxel is used or is for use at a dosage disclosed herein.

In exemplary embodiments, the anti-clusterin antibody or antigen binding fragment thereof is used or is for use at a dose of 12 mg/kg once weekly and docetaxel is used at a dose of 75 mg/m² once every three weeks.

In exemplary embodiments the anti-clusterin antibody or antigen binding fragment thereof is used or is for use at a dose of 12 mg/kg once weekly and docetaxel is used at a dose of 60 mg/m² once every three weeks.

In exemplary embodiments the anti-clusterin antibody or antigen binding fragment thereof is used or is for use at a dose of 9 mg/kg once weekly and docetaxel is used at a dose of 75 mg/m² once every three weeks.

In exemplary embodiments the anti-clusterin antibody or antigen binding fragment thereof is used or is for use at a dose of 9 mg/kg once weekly and docetaxel is used at a dose of 60 mg/m² once every three weeks.

In exemplary embodiments the anti-clusterin antibody or antigen binding fragment thereof is used or is for use at a dose of 6 mg/kg once weekly and docetaxel is used at a dose of 75 mg/m² once every three weeks.

In exemplary embodiments the anti-clusterin antibody or antigen binding fragment thereof is used or is for use at a dose of 6 mg/kg once weekly and docetaxel is used at a dose of 60 mg/m² once every three weeks.

In exemplary embodiments the anti-clusterin antibody or antigen binding fragment thereof is used or is for use at a dose of 3 mg/kg once weekly and docetaxel is used at a dose of 75 mg/m² once every three weeks.

In exemplary embodiments the anti-clusterin antibody or antigen binding fragment thereof is used or is for use at a dose of 3 mg/kg once weekly and docetaxel is used at a dose of 60 mg/m² once every three weeks.

The present disclosure also provides a kit comprising an anti-cancer therapy for use in a method as described herein and for treating a subject as described herein.

In some embodiments, the kit comprises one or more containers comprising at least one dose of an anti-clusterin antibody or antigen binding fragment thereof, one or more containers comprising at least one dose of a chemotherapeutic for use in combination therapy and a package insert comprising instructions for treating a subject in need.

The present disclosure also provides a kit comprising one or more containers comprising at least one dose of an anti-clusterin antibody or antigen binding fragment thereof, one or more containers comprising at least one dose of docetaxel for use in combination therapy and a package insert comprising instructions for treating a subject in need.

The kit of the present disclosure comprises the anti-clusterin antibody or antigen binding fragment thereof disclosed herein.

In some embodiments, the package insert states that the combination therapy is intended for treatment of a subject having a carcinoma.

In some embodiments, the package insert states that the combination therapy is intended for treatment of a subject having a carcinoma that is metastatic.

In some embodiments, the package insert states that the combination therapy is intended for treatment of a subject in need as disclosed herein.

In some embodiments, the package insert states that the combination therapy is intended for treatment of a subject having a carcinoma that progressed after a first line immune checkpoint therapy.

In some embodiments, the package insert states that the combination therapy is intended for treatment of a subject having a carcinoma that has failed prior treatment with an immune checkpoint therapy and a platinum-containing doublet treatment (e.g., simultaneously or sequentially).

In some embodiments, the package insert states that the combination therapy is intended for treatment of a subject having a carcinoma that has failed prior treatment with an anti-PD1 or PD-L1 immune checkpoint antibody and a platinum-containing doublet treatment (e.g., simultaneously or sequentially).

In some embodiments, the package insert states that the combination therapy is intended for treatment of a subject having non-small cell lung cancer (NSCLC), such as advanced NSCLC, stage III NSCLC and/or stage IV NSCLC.

In some embodiments, the package insert states that the combination therapy is intended for treatment of a subject having breast cancer.

In some embodiments, the package insert states that the combination therapy is intended for treatment of a subject having metastatic breast cancer.

In some embodiments, the package insert states that the combination therapy is intended for treatment of a subject having prostate cancer.

In some embodiments, the package insert states that the combination therapy is intended for treatment of a subject having metastatic prostate cancer.

In some embodiments, the package insert states that the combination therapy is intended for treatment of a subject having gastric cancer.

In some embodiments, the package insert states that the combination therapy is intended for treatment of a subject having metastatic gastric cancer.

In some embodiments, the package insert states that the combination therapy is intended for treatment of a subject having head and neck cancer.

In some embodiments, the package insert states that the combination therapy is intended for treatment of a subject having metastatic head and neck cancer.

In some embodiments, the package insert states that the combination therapy is intended for treatment of a subject having thyroid cancer.

In some embodiments, the package insert states that the combination therapy is intended for treatment of a subject having metastatic thyroid cancer.

In some embodiments, the package insert states that the combination therapy is intended for treatment of a subject having ovarian cancer.

In some embodiments, the package insert states that the combination therapy is intended for treatment of a subject having metastatic ovarian cancer.

In some embodiments, the package insert states that the combination therapy is intended for treatment of a subject having endometrial cancer.

In some embodiments, the package insert states that the combination therapy is intended for treatment of a subject having metastatic endometrial cancer.

In some embodiments, the package insert states that the combination therapy is intended for treatment of a subject having liver cancer.

In some embodiments, the package insert states that the combination therapy is intended for treatment of a subject having metastatic liver cancer.

In some embodiments, the package insert states that the combination therapy is intended for treatment of a subject having colorectal cancer.

In some embodiments, the package insert states that the combination therapy is intended for treatment of a subject having metastatic colorectal cancer.

In some embodiments, the package insert states that the combination therapy is intended for treatment of a subject having pancreatic cancer.

In some embodiments, the package insert states that the combination therapy is intended for treatment of a subject having metastatic pancreatic cancer.

In some embodiments, the package insert states that the combination therapy is intended for treatment of a subject having cholangiocarcinoma.

In some embodiments, the package insert states that the combination therapy is intended for treatment of a subject having metastatic cholangiocarcinoma.

In some embodiments, the package insert states that the combination therapy is intended for treatment of a subject having mesothelioma.

In some embodiments, the package insert states that the combination therapy is intended for treatment of a subject having metastatic mesothelioma.

In some embodiments, the package insert states that the combination therapy is intended for treatment of a subject having melanoma.

In some embodiments, the package insert states that the combination therapy is intended for treatment of a subject having metastatic melanoma.

In some embodiments, the package insert states that the combination therapy is intended for treatment of a subject having bladder cancer.

In some embodiments, the package insert states that the combination therapy is intended for treatment of a subject having metastatic bladder cancer.

In some embodiments, the package insert states that the combination therapy is intended for treatment of a subject having cervical cancer.

In some embodiments, the package insert states that the combination therapy is intended for treatment of a subject having metastatic cervical cancer.

In some embodiments, the package insert states that the combination therapy is intended for treatment of a subject that is not immunosuppressed has not received an immunosuppressive medication within 7 days prior to treatment.

In some embodiments, the package insert states that the combination therapy is intended for treatment of a subject that has not received prior treatment with docetaxel.

In some embodiments, the package insert states that the combination therapy is for administration essentially over the entire course of the treatment period (e.g., throughout the treatment period).

The present disclosure also relates to a kit comprising one or more containers comprising at least one dose of the medicament disclosed herein and a package insert as disclosed herein comprising instructions for treating a subject in need, wherein the anti-clusterin antibody or antigen binding fragment thereof and docetaxel are provided in separate containers.

### BRIEF DESCRIPTION OF THE DRAWINGS

FIG. 1: 4T1 Lung Metastases Are Immunologically “Cold” Which Prevents Immune Lymphocyte Infiltration. CD3+ and CD8+ T cells are present in the margins of 4T1 lung metastases resulting from the creation of a restrictive tumor microenvironment as a consequence of the epithelial to mesenchymal transitions that prevents lymphocytic infiltration.

FIG. 2A: Inhibition of EMT with the 16B5 Anti-sCLU mAb Results in B (B220) and T (CD3, CD4, CD8) Lymphocytes Infiltration in 4T1 Lung Metastases.

FIG. 2B: Picture of human tumor biopsies of patients treated with AB-16B5 as single agent.

FIG. 3: Graph of the number of lung nodules in 4T1-implanted animals treated with AB-16B5 in monotherapy or in combination with docetaxel.

FIG. 4A and FIG. 4B: 4T1 lung metastases from animals treated with AB-16B5 in monotherapy or in combination with docetaxel are infiltrated by B and T lymphocytes. 4T1 lung metastases were dissected at Day 36 post-implantation and processed with collagenase and hyaluronidase for immunophenotyping by flow cytometry.

FIG. 5: chart showing the duration of patients on treatment as the best response as per RECIST 1.1 (PT: Patient, BR: Best response, PD-L1: PD-L1 tumor proportion score, PR; Partial response, SD: Stable disease, PD: progressive disease, AE; adverse event).

FIG. 6: histology analyses of pre-(a and c) and on-treatment (b and d) biopsies from two patients under study. Tumor sections were stained with hematoxylin and eosin.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### DETAILED DESCRIPTION

### Definitions

Unless indicated otherwise, the amino acid numbering indicated for the dimerization domain are in accordance with the EU numbering system.

The use of the terms “a” and “an” and “the” and similar referents in the context of describing embodiments (especially in the context of the claims) are to be construed to cover both the singular and the plural, unless otherwise indicated herein or clearly contradicted by context.

Unless specifically stated or obvious from context, as used herein the term “or” is understood to be inclusive and covers both “or” and “and”.

The term “and/or” where used herein is to be taken as specific disclosure of each of the specified features or components with or without the other.

The terms “comprising”, “having”, “including”, and “containing” are to be construed as open-ended terms (i.e., meaning “including, but not limited to”) unless otherwise noted. The term “consisting of” is to be construed as close-ended.

The term “treatment” for purposes of this disclosure refers to both therapeutic treatment and prophylactic or preventative measures. Those in need of treatment include those already with the disorder as well as those prone to have the disorder or those in whom the disorder is to be prevented.

The term “EMT signature” as used herein refers to changes that are indicative of a loss of epithelial phenotype and/or acquisition of a mesenchymal phenotype that are observable at the cellular level and/or observable or measurable at the genetic level or protein level.

The term “about” or “approximately” with respect to a given value means that variation in the value is contemplated. In some embodiments, the term “about” or “approximately” shall generally mean a range within +/−20 percent, within +/−10 percent, within +/−5 percent, within +/−4 percent, within +/−3 percent, within +/−2 percent or within +/−1 percent of a given value or range.

The expression “over the entire course of the treatment period” means that both the anti-clusterin antibody or antigen binding fragments and docetaxel are administered at each treatment cycle.

The term “essentially” is used to characterize an action that is carried out most of the time or a state that occurs most of the time. For example, the expression “essentially over the entire course of the treatment period” means that both the anti-clusterin antibody or antigen binding fragments and docetaxel are administered at each treatment cycle and during the entire treatment period but occasionally a dose of either of the anti-clusterin antibody or antigen binding fragments or docetaxel or a dose of each may be intentionally or non-intentionally missed.

The term “functional immune system” with respect to a subject means that the immune system of the subject is essentially not affected by cancer or by medication or that the subject is not immunosuppressed.

The expression “treatment comprising an immune checkpoint antibody” refers to treatment with an immune checkpoint antibody as monotherapy or in combination therapy.

The expression “adequate organ and immune function” refers to one or more of the parameters provided in Table 7.

### Methods and Uses

In some aspects and embodiments, the present disclosure provides a method for allowing infiltration of immune cells in the tumor microenvironment.

In some embodiments, the method of the present disclosure is used for treating a subject having cancer.

In vet other aspects and embodiments, the present disclosure provides a method for treating a subject having cancer that comprises promoting infiltration of immune cells in the tumor microenvironment by administering an anti-cancer therapy that comprises an anti-clusterin antibody or an antigen binding fragment thereof to a subject in need thereof.

In exemplary embodiments, the anti-clusterin antibody or an antigen binding fragment thereof is administered as a single agent to a subject in need thereof.

In other exemplary embodiments, the anti-clusterin antibody or an antigen binding fragment thereof may be used in combination therapy as described herein.

The method of the present disclosure may also comprise administering other therapy for improving the antitumor immune response. In some exemplary embodiments, the method may comprise administering cytokines and/or chemokines.

It is to be understood herein, that the anti-clusterin antibody or an antigen binding fragment thereof may be used as a single agent for a desired period of time and then used in combination therapy.

In some embodiments, the method of the present disclosure is for treating a subject as disclosed herein.

For example, the method is for treating a human subject. In some instances, the method is used for example, for treating an adult (i.e., ≥18 years of age).

In some instances, the method comprises administering an anti-cancer therapy that comprises the anti-clusterin antibody or antigen binding fragment thereof in combination with an anti-cancer agent.

In exemplary embodiments, the method comprises administering an anti-clusterin antibody or antigen binding fragment thereof in conjunction with radiation therapy or chemotherapy.

In particular embodiments, the method comprises administering an anti-clusterin antibody or antigen binding fragment thereof in combination with a chemotherapeutic.

In some instances, the method of the present disclosure may be used to overcome or lessen chemotherapeutic resistance mediated by MDR1/P-glycoprotein. Accordingly, the combination therapy may comprise a chemotherapeutic associated with resistance mediated by MDR1/P-glycoprotein.

In some exemplary embodiments, the method may comprise administering an anti-clusterin antibody or antigen binding fragment thereof and a chemotherapeutic including for example, an alkylating agent, an anti-metabolite, an alkaloid, an anti-tumor antibiotic or combination thereof.

In some instances, the method may comprise administering an anti-clusterin antibody or antigen binding fragment thereof and an alkylating agent selected, for example, from altretamine, busulfan, carboplatin, carmustine, cisplatin, cyclophosphamide, dacarbazine, ifosfamide, lomustine, melphalan, temozolomide, trabectedin or derivatives or analogs thereof.

In other instances, the method may comprise administering an anti-clusterin antibody or antigen binding fragment thereof and an anti-metabolite selected, for example, 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, trifluridine, tipiracil or derivatives or analogs thereof.

In yet other instances, the method may comprise administering an anti-clusterin antibody or antigen binding fragment thereof and an alkaloid selected, for example, from vincristine, vinblastine, vinorelbine, taxanes, etoposide, teniposide, irinotecan, topotecan or derivatives or analogs thereof.

In some instances, the method may comprise administering an anti-clusterin antibody or antigen binding fragment thereof and a taxane selected from docetaxel, paclitaxel and derivatives or analogues including for example and without limitations, Abraxane®, Cabazitaxel, larotaxel, milataxel, ortataxel, tesetaxel and others.

In an exemplary embodiment, the method may comprise administering an anti-clusterin antibody or antigen binding fragment thereof and docetaxel.

In another exemplary embodiment, the method may comprise administering an anti-clusterin antibody or antigen binding fragment thereof and paclitaxel.

In other instances, the method may comprise administering an anti-clusterin antibody or antigen binding fragment thereof and an anti-tumor antibiotic selected, for example, from daunorubicin, doxorubicin, doxorubicin liposomal, epirubicin, idarubicin, valrubicin, derivatives or analogs thereof.

For example, the anti-clusterin antibody or an antigen binding fragment thereof may be used in combination with docetaxel such as to generate chemotherapy-induced immunogenic modulation.

In some embodiments, the method of the present disclosure more particularly comprises administering an anti-clusterin antibody or an antigen binding fragment thereof in combination with docetaxel to a subject in need thereof.

In some exemplary embodiments, the method is for treating carcinoma including for example and without limitations, endometrial cancer, breast cancer, liver cancer, prostate cancer, renal cancer, bladder cancer, cervical cancer, ovarian cancer, colorectal cancer, pancreatic cancer, lung cancer, gastric cancer, head and neck cancer, thyroid cancer, cholangiocarcinoma, mesothelioma or melanoma.

In further exemplary embodiments, the method is for treating metastatic carcinoma including for example and without limitations, metastatic endometrial cancer, metastatic breast cancer, metastatic liver cancer, metastatic prostate cancer, metastatic renal cancer, metastatic bladder cancer, cervical cancer, metastatic ovarian cancer, metastatic colorectal cancer, metastatic pancreatic cancer, metastatic lung cancer, metastatic gastric cancer, metastatic head and neck cancer, metastatic thyroid cancer, metastatic cholangiocarcinoma, metastatic mesothelioma or metastatic melanoma.

In some embodiment, the method is for treating a subject having carcinoma or metastatic carcinoma associated with one or more lesions having low expression of PD-L1, no evidence of PD-L1 expression or that is not eligible to treatment comprising an anti-PD-1 or anti-PD-L1 immune checkpoint antibody.

In some embodiments the method is for treating a subject that is not eligible to treatment comprising an anti-PD-1 or anti-PD-L1 immune checkpoint inhibitor or that would unlikely benefit from treatment comprising an anti-PD-1 or anti-PD-L1 immune checkpoint inhibitor.

Accordingly, the method of the present disclosure is for treating a subject having carcinoma or metastatic carcinoma characterized by one or more lesions with low expression of PD-L1, no evidence of PD-L1 expression or that is not eligible to treatment comprising an anti-PD-1 or anti-PD-L1 immune checkpoint antibody and wherein the carcinoma or method are as described herein and/or exemplified below.

The present disclosure also relates, in further aspect thereof, to a method of treating a subject having a carcinoma that would unlikely benefit from treatment comprising an anti-PD-1 or anti-PD-L1 immune checkpoint antibody.

The present disclosure also relates to a method for allowing infiltration of immune cells in one or more lesions characterized as immunologically cold and further characterized as having low expression of programmed death ligand 1 (PD-L1), no evidence of PD-L1 expression or that is not treatable with an anti-PD-1 or anti-PD-L1 immune checkpoint antibody (e.g., monotherapy, in combination with chemotherapy etc.), the method comprising administering an anti-cancer therapy that comprises an anti-clusterin antibody or antigen binding fragment thereof to a subject having the one or more lesions.

In some embodiments, the method of the present disclosure is for treating a subject having a carcinoma with one or more lesions having a PD-L1 tumor proportion score (TPS) of ≥50%.

In some embodiments, the method of the present disclosure is for treating a subject having a carcinoma with one or more lesions having a PD-L1 tumor proportion score (TPS) of <50%.

In some embodiments, the method of the present disclosure is for treating a subject having a carcinoma with one or more lesions having a PD-L1 tumor proportion score (TPS) of between 1 to 49%.

In some aspects, the method of the present disclosure is for treating a subject having carcinoma having one or more lesions having a PD-L1 tumor proportion score (TPS) of ≤15%.

In other aspects, the method of the present disclosure for treating a subject having carcinoma having one or more lesions have a PD-L1 tumor proportion score (TPS) of <5%.

In some aspects, the method of the present disclosure is for treating a subject having carcinoma having one or more lesions having a PD-L1 tumor proportion score (TPS) of ≤1%.

In additional aspects, the method of the present disclosure is for treating a subject having carcinoma having one or more lesions have a PD-L1 tumor proportion score (TPS) of <1%.

In other aspects, the method of the present disclosure is for treating a subject having carcinoma having one or more lesions have a PD-L1 combined positive score (CPS) of <10%.

In other aspects, the method of the present disclosure is for treating a subject having carcinoma having one or more lesions have a PD-L1 combined positive score (CPS) of <5%.

In yet other aspects, the method of the present disclosure is for treating a subject having carcinoma having one or more lesions have a PD-L1 combined positive score (CPS) of ≤1%.

In yet other aspects, the method of the present disclosure is for treating a subject having carcinoma having one or more lesions have a PD-L1 combined positive score (CPS) of <1%.

In further aspects, the method of the present disclosure is for treating a subject having carcinoma in which PD-L1 expression is undetectable or non-evaluable.

In some aspects and embodiments, the method of the present disclosure may promote infiltration of immune cells in the tumor microenvironment.

In some instances, the method of the present disclosure may be used to modulate an antitumor immune response.

It some embodiments, the modulation of an antitumor immune response may include an increase in the immune response against tumor cells. In some instances, the method of the present disclosure may induce tumor regression. In other instances, the method of the present disclosure may induce stabilization of tumor growth.

In some embodiments, the method of the present disclosure is for treating a subject having a carcinoma characterized by having one or more lesions that are immunologically cold.

In some instances, the lesions that are immunologically cold may also have low expression of PD-L1 or no evidence of PD-L1 expression.

In other embodiments, the method of the present disclosure is for treating a subject having a carcinoma characterized by having one or more lesions that have an epithelial to mesenchymal transition (EMT) signature.

In other embodiments, the method of the present disclosure is for treating a subject having a carcinoma characterized by having one or more lesions that show signs of an EMT signature.

In yet other embodiments, the method of the present disclosure is for treating a subject having a carcinoma characterized by having one or more lesions carrying a KRAS mutation.

In some embodiments, the method is for treating a subject having non-small cell lung cancer (NSCLC).

In exemplary embodiments, the method is for treating a subject having metastatic NSCLC.

In other exemplary embodiments, the method is for treating a subject having stage III NSCLC.

In other exemplary embodiments, the method is for treating a subject having stage IV NSCLC.

In other exemplary embodiments, the method is for treating a subject having stage III to IV NSCLC.

In some embodiments, the method is for treating a subject having lung adenocarcinoma.

In some embodiments, the method is for treating a subject having squamous cell lung cancer.

In some embodiments, the method is for treating a subject having large cell lung carcinoma.

In some embodiments, the method is for treating a subject having adenosquamous lung carcinoma.

In some embodiments, the method is for treating a subject having non-small cell lung cancer (e.g., metastatic non-small cell lung cancer) characterized has having a KRAS mutation.

In other embodiments, the method is for treating a subject having breast cancer.

In yet other embodiments, the method is for treating a subject having metastatic breast cancer.

In further embodiments, the method is for treating a subject having prostate cancer.

In other embodiments, the method is for treating subject having metastatic prostate cancer.

In additional embodiments, the method is for treating subject having bladder cancer.

In yet additional embodiments, the method is for treating subject having metastatic bladder cancer.

In exemplary embodiments, the method is for treating subject having cervical cancer.

In other exemplary embodiments, the method is for treating subject having metastatic cervical cancer.

In some embodiments, the method is for treating a subject having gastric cancer.

In other embodiments, the method is for treating a subject having metastatic gastric cancer.

In additional embodiments, the method is for treating a subject having head and neck cancer.

In further embodiments, the method is for treating a subject having metastatic head and neck cancer.

In yet further embodiments, the method is for treating a subject having thyroid cancer.

In some embodiments, the method is for treating a subject having metastatic thyroid cancer.

In some embodiments, the method is for treating a subject having ovarian cancer.

In other embodiments, the method is for treating a subject having metastatic ovarian cancer.

In further embodiments, the method is for treating a subject having endometrial cancer.

In yet further embodiments, the method is for treating a subject having metastatic endometrial cancer.

In some embodiments, the method is for treating a subject having liver cancer.

In other embodiments, the method is for treating a subject having metastatic liver cancer.

In some embodiments, the method is for treating a subject having colorectal cancer.

In additional embodiments, the method is for treating a subject having metastatic colorectal cancer.

In other embodiments, the method is for treating a subject having pancreatic cancer.

In other embodiments, the method is for treating a subject having metastatic pancreatic cancer.

In some embodiments, the method is for treating a subject having cholangiocarcinoma.

In further embodiments, the method is for treating a subject having metastatic cholangiocarcinoma.

In yet further embodiments, the method is for treating a subject having mesothelioma.

In some embodiments, the method is for treating a subject having metastatic mesothelioma.

In exemplary embodiments, the method is for treating a subject having melanoma.

In other exemplary embodiments, the method is for treating a subject having metastatic melanoma.

In some exemplary embodiments, the method is for treating a subject that has not received prior treatment comprising an anti-PD-1 or anti-PD-L1 immune checkpoint antibody.

In other exemplary embodiments, the method is for treating a subject that has received prior treatment comprising an anti-PD-1 or anti-PD-L1 immune checkpoint antibody.

In other exemplary embodiments, the method is for treating a subject that has received prior treatment comprising an anti-PD-1 or anti-PD-L1 immune checkpoint antibody and chemotherapy.

In other exemplary embodiments, the method is for treating a subject that has received prior treatment comprising an anti-PD-1 or anti-PD-L1 immune checkpoint antibody and a chemotherapeutic doublet treatment.

In accordance with the present disclosure, the doublet treatment may be provided simultaneously.

In accordance with the present disclosure, the doublet treatment may be provided sequentially.

In accordance with the present disclosure the method is for treating a subject having a carcinoma that progressed after a prior treatment.

For example, the method is for treating a subject having a carcinoma that progressed after a prior treatment that comprises an anti-PD-1 or anti-PD-L1 immune checkpoint antibody.

In accordance with the present disclosure the method is for treating a subject having a carcinoma that progressed after a prior treatment that comprises an anti-PD-1 or anti-PD-L1 immune checkpoint antibody.

In other exemplary embodiments, the method is for treating a subject that has failed prior treatment comprising an anti-PD-1 or anti-PD-L1 immune checkpoint antibody.

In other exemplary embodiments, the method is for treating a subject that has failed prior treatment comprising an anti-PD-1 or anti-PD-L1 immune checkpoint antibody and chemotherapy.

In other exemplary embodiments, the method is for treating a subject for which prior treatment comprises an anti-PD-1 immune checkpoint antibody.

In other exemplary embodiments, the method is for treating a subject for which prior treatment comprises an anti-PD-L1 immune checkpoint antibody.

In other exemplary embodiments, the method is for treating a subject for which prior treatment comprises, for example and without limitations, from ipilimumab, nivolumab, pembrolizumab, cemiplimab, atezolizumab, avelumab, or durvalumab.

In other exemplary embodiments, the method is for treating a subject for which prior treatment comprises pembrolizumab.

In other exemplary embodiments, the method is for treating a subject that is not eligible to treatment with ipilimumab, nivolumab, pembrolizumab, cemiplimab, atezolizumab, avelumab, or durvalumab.

In particular embodiments, the method is for treating a subject that is not eligible to treatment with pembrolizumab.

In other exemplary embodiments, the method is for treating a subject that has an adequate organ and/or immune function.

In other exemplary embodiments, the method is for treating a subject that has a functional immune system.

In some embodiments, the method comprises administering an anti-clusterin antibody or antigen binding fragment thereof that is able to inhibit epithelial to mesenchymal transition of carcinoma cells.

In some embodiments, the method comprises administering an anti-clusterin antibody or antigen binding fragment thereof that has the amino acid sequence set forth in Table 9.

In some embodiments, the method comprises administering an anti-clusterin antibody or antigen binding fragment thereof that has the variable regions of the humanized 16B5 antibody. In accordance with the present disclosure, the method comprises administering an anti-cancer therapy comprising the anti-clusterin antibody or antigen binding fragment thereof as a first-line therapy.

In accordance with the present disclosure, the method comprises administering an anti-cancer therapy comprising the anti-clusterin antibody or antigen binding fragment thereof as a second-line therapy.

In accordance with the present disclosure, the method comprises administering an anti-cancer therapy comprising the anti-clusterin antibody or antigen binding fragment thereof as a subsequent therapy (e.g., third-line or more).

In some embodiments, the method of the present disclosure may comprise treating a subject having an early-stage cancer.

In other embodiments, the method of the present disclosure may comprise treating a subject having a late-stage cancer.

The present disclosure also relates to a method of treating cancer by administering the anti-cancer therapy as a neoadjuvant therapy. In accordance with the present disclosure, the method comprises administering the anti-cancer therapy prior to tumor-resection.

The present disclosure also relates to a method of treating cancer by administering the anti-cancer therapy as an adjuvant therapy. In accordance with the present disclosure, the method comprises administering the anti-cancer therapy after to tumor-resection.

The method of the present disclosure may comprise administering the anti-cancer therapy of the present disclosure prior to and after tumor resection.

In other aspects and embodiments, the present disclosure relates to the use of an anti-clusterin antibody or an antigen binding fragment thereof for allowing infiltration of immune cells in a tumor (e.g., solid tumor) microenvironment in a subject in need thereof.

In yet other aspects and embodiments, the present disclosure relates to the use of an anti-clusterin antibody or an antigen binding fragment thereof in the manufacture of a medicament or kit for allowing infiltration of immune cells in a tumor (e.g., solid tumor) microenvironment in a subject in need thereof.

In additional aspects and embodiments, the present disclosure relates to the use of an anti-clusterin antibody or antigen binding fragment thereof in the treatment of a subject having cancer (e.g., a solid tumor).

In yet additional aspects and embodiments, the present disclosure relates to the use of an anti-clusterin antibody or antigen binding fragment thereof in the manufacture of a medicament or kit for the treatment of a subject having cancer (e.g., a solid tumor).

In additional aspects and embodiments, the present disclosure relates to the use of a combination therapy comprising an anti-clusterin antibody or antigen binding fragment thereof and docetaxel in the treatment of a subject having cancer (e.g., a solid tumor).

In yet additional aspects and embodiments, the present disclosure relates to the use of an anti-clusterin antibody or antigen binding fragment thereof and docetaxel in the manufacture of a medicament or kit for the treatment of a subject having cancer (e.g., a solid tumor).

In some embodiments, the subject in need is a subject having cancer and a functional immune system.

In some embodiments, the subject in need is a subject having cancer and an adequate organ and immune function.

In some embodiments, the method of the present disclosure may result in an increase (in the presence or in the quantity) of immune cells in the tumor microenvironment.

In some embodiments, the method or use of the present disclosure may result in infiltration of immune cells in a primary tumor microenvironment.

In some embodiments, the tumor microenvironment may be infiltrated with immune cells such as plasmocytes.

In some embodiments, method or use of the present disclosure may result in infiltration of T cells in the tumor microenvironment.

In some embodiments, method or use of the present disclosure may result in infiltration of CD4⁺ T cells in the tumor microenvironment.

In some embodiments, method or use of the present disclosure may result in infiltration of CD8⁺ T cells in the tumor microenvironment.

In some embodiments, method or use of the present disclosure may result in infiltration of B cells in the tumor microenvironment.

In some embodiments, the absence or presence of immune cells in the tumor microenvironment may be confirmed by tumor biopsy.

In other embodiments, the absence or presence of immune cells in the tumor microenvironment may be confirmed by in vivo imaging (e.g., magnetic resonance imaging, e.g., see Jiang X. et al., 2020).

A “cold tumor” includes for example, a tumor that is not likely to trigger a strong immune response. Cold tumors tend to be surrounded by cells that are able to suppress the immune response and keep T cells from attacking the tumor cells and killing them. Cold tumors usually do not respond to immunotherapy (National Cancer Institute website).

A tumor may be characterized as “immunologically cold” when the tumor microenvironment is not sufficiently infiltrated by immune cells (especially by lymphocytes) or when the tumor microenvironment is not inflamed. In contrast, a tumor may be characterized as “immunologically warm” or “immunologically hot” when infiltration of immune cells (especially by lymphocytes) in the tumor microenvironment is observed or when the tumor shows sign of inflammation.

Generally, a pathologist, a technologist, a trained scientist or trained technician equipped with proper reagents and/or apparatus may be able to determine the absence or presence of immune cells in the tumor microenvironment and may thus be able to evaluate whether a tumor is “immunologically cold”, “immunologically warm” or “immunologically hot”.

Liquid biopsy may also represent a valuable approach to determine tumor immunogenicity. For example, low tumor immunogenicity may provide indications that the patient carries an immunologically cold tumor.

Liquid biopsies offer the possibility to assess overall tumor burden by monitoring the levels of circulating tumor DNA (ctDNA) or by quantifying the circulating tumor cells (CTCs). Both approaches allow assessments of tumor genome instability through determination of microsatellite stability or by identifying driver mutations in oncogenes. Tumor cells with microsatellite stability predict lower numbers of neoantigens that contribute to lower immunogenicity. It is also possible to monitor gene signatures in RNA isolated from CTCs. CTCs with partial EMT or EMT-high signatures may predict worse outcomes for patients including those treated with immunotherapies as EMT contributes to the generation of a “cold” or restrictive tumor microenvironment.

Single agent or combination therapy may thus be administered subsequent to confirmation that the subject has a “immunologically cold tumor”.

In addition, detection of immune cells in the tumor microenvironment may reveal that treatment with anti-clustering antibody or antigen binding fragment thereof as a single agent or in combination therapy with docetaxel effectively allows infiltration of immune cells in a the tumor microenvironment.

The method or use of the present disclosure may result in the tumor being more susceptible to treatment by immunotherapy. The present disclosure therefore includes a step of administering immunotherapy after one or more cycle of the anti-clusterin antibody or antigen binding fragment thereof as single agent or in combination therapy with docetaxel.

The immunotherapy includes for example, immune checkpoint inhibitors (anti-PD1 or anti-PDL-1 antibodies, anti-CTL-A4 antibodies) and cellular immunotherapy (e.g., CAR-T cells, TILs).

Exemplary embodiments of FDA approved immune checkpoint inhibitors include ipilimumab, nivolumab, pembrolizumab, cemiplimab, atezolizumab, avelumab, and durvalumab.

In some embodiments, the method or use of the present disclosure may result in modulation of an immune response towards tumor cells.

The method or use of the present disclosure may result in an increased immune response towards tumor cells.

In some embodiments, the method or use of the present disclosure may result in necrosis of a tumor.

In other aspects and embodiments, the present disclosure relates to a method of treating a subject having cancer by administering an anti-clusterin antibody or antigen binding fragment thereof. The subject may have a functional immune system.

In yet other aspects and embodiments, the present disclosure relates to a method of treating a subject having cancer by administering a combination therapy comprising an anti-clusterin antibody or antigen binding fragment thereof and docetaxel. The subject may have a functional immune system. The subject may have an adequate organ and immune function.

In some embodiments, the method or use of the present disclosure does not require concurrent anti-cancer treatment during the treatment period.

In some embodiments, the method or use of the present disclosure does not involve concurrent anti-cancer treatment during the treatment period.

In accordance with the present disclosure the method or use comprises administering an anti-clusterin antibody or antigen binding fragment thereof at a dose of between approximately 3 mg/kg to approximately 20 mg/kg.

Also, in accordance with the present disclosure the method or use comprises administering an anti-clusterin antibody or antigen binding fragment thereof at a dose of between approximately 3 mg/kg to approximately 20 mg/kg and docetaxel at a dose of between approximately 60 mg/m² to approximately 100 mg/m².

The anti-clusterin antibody or antigen binding fragment thereof and docetaxel are generally administered on same day. However, it is possible that they be administered on a different day.

The anti-clusterin antibody or antigen binding fragment thereof and docetaxel are both administered over the entire course of the treatment period. In some embodiments, the anti-clusterin antibody or antigen binding fragment thereof and docetaxel are both administered at each cycle.

The anti-clusterin antibody or antigen binding fragment thereof and docetaxel are both essentially administered over the entire course of the treatment period. In some embodiments, the anti-clusterin antibody or antigen binding fragment thereof and docetaxel are administered at all cycles.

The anti-clusterin antibody or antigen binding fragment thereof and docetaxel are both generally administered at each treatment cycle. However, it is possible that one or more doses of the anti-clusterin antibody or antigen binding fragment thereof and/or docetaxel is missed without negatively impacting the treatment. It is also possible that one or more additional doses of the anti-clusterin antibody or antigen binding fragment thereof and/or docetaxel is administered without negatively impacting the treatment.

The method or use may also involve interrupting treatment (single agent or combination therapy) for a period of time (e.g., for one week, two weeks, three weeks, four weeks, five weeks, six weeks, seven weeks, eight weeks, nine weeks, ten weeks, at least ten weeks, one month, two months, three months, four months, five months, six months, seven months, eight months, nine months, ten months, eleven months, at least six months, at least ten months, at least one year, once cycle, two cycles, three cycles, four cycles, five cycles, six cycles, seven cycles, eight cycles, nine cycles, ten cycles, at least ten cycles). Treatment may be reinitiated afterward.

In some embodiments, at least one treatment cycle is interrupted.

In other embodiments, more than one treatment cycles are interrupted.

In some embodiments the interruption lasts from one day to one week. In some embodiments the interruption lasts from one day to two weeks. In some embodiments the interruption lasts from one day to three weeks. In some embodiments the interruption lasts from one day to one month. In some embodiments the interruption lasts from one day to two months. In some embodiments the interruption lasts from one day to three months. In some embodiments the interruption lasts from one day to four months. In some embodiments the interruption lasts from one day to five months. In some embodiments the interruption lasts from one day to six months. In some embodiments the interruption lasts from one day to more than six months.

In some embodiments, the method or use may involve treating a subject in need with the anti-clusterin antibody or antigen binding fragment thereof and docetaxel combination therapy for one or more cycles and subsequently treating the subject with the anti-clusterin antibody or antigen binding fragment thereof as a single agent.

In some embodiments, the method or use may involve treating a subject in need with the anti-clusterin antibody or antigen binding fragment thereof as a single agent and subsequently treating the subject with the anti-clusterin antibody or antigen binding fragment thereof and docetaxel combination therapy for one or more cycles.

In accordance with the present disclosure, the method or use is for the treatment of a carcinoma in a subject in need thereof.

In accordance with the present disclosure, the method or use is for the treatment of a metastatic carcinoma in a subject in need thereof.

In some embodiments, the cancer is non-small cell lung cancer (NSCLC). In an exemplary embodiment, the cancer is metastatic NSCLC such as stage III to IV NSCLC.

In some embodiments, the cancer is breast cancer. In an exemplary embodiment, the cancer is metastatic breast cancer.

In some embodiments, the cancer is prostate cancer. In an exemplary embodiment, the cancer is metastatic prostate cancer.

In some embodiments, the cancer is gastric cancer. In an exemplary embodiment, the cancer is metastatic gastric cancer.

In some embodiments, the cancer is head and neck cancer. In an exemplary embodiment, the cancer is metastatic head and neck cancer.

In some embodiments, the cancer is thyroid cancer. In an exemplary embodiment, the cancer is metastatic thyroid cancer.

In some embodiments, the cancer is ovarian cancer. In an exemplary embodiment, the cancer is metastatic ovarian cancer.

In other embodiments, the present disclosure relates to the use of the anti-cancer therapy disclosed herein as an adjuvant therapy.

In yet other embodiments, the present disclosure relates to the use of the anti-cancer therapy disclosed herein as a neo-adjuvant therapy.

### Dosages, Treatment Regimens and Schedules

### Anti-Clusterin Antibody

In accordance with the present disclosure, the anti-clusterin antibody or antigen binding fragment thereof is administered at a dose sufficient to result in infiltration of immune cells in the tumor microenvironment.

In some embodiments, the dose of the anti-clusterin antibody or antigen binding fragment thereof is a therapeutically effective and safe dose.

In accordance with the present disclosure, the anti-clusterin antibody or antigen binding fragment thereof is administered at an administration interval sufficient to result in infiltration of immune cells in the tumor microenvironment.

In accordance with the present disclosure, the anti-clusterin antibody or antigen binding fragment thereof is administered for a treatment period sufficient to result in infiltration of immune cells in the tumor microenvironment.

In some embodiments, the anti-clusterin antibody or antigen binding fragment thereof is administered at a dose, administration interval and/or treatment period sufficient to result in infiltration of immune cells in the tumor microenvironment.

In accordance with an exemplary embodiment of the disclosure, the anti-clusterin antibody or antigen binding fragment thereof is administered once weekly.

In accordance with another exemplary embodiment of the disclosure, the anti-clusterin antibody or antigen binding fragment thereof is administered twice weekly.

In accordance with yet another exemplary embodiment of the disclosure, the anti-clusterin antibody or antigen binding fragment thereof is administered thrice weekly.

In accordance with a further exemplary embodiment of the disclosure, the anti-clusterin antibody or antigen binding fragment thereof is administered once every two weeks.

In accordance with yet a further exemplary embodiment of the disclosure, the anti-clusterin antibody or antigen binding fragment thereof is administered once every three weeks.

In accordance with an additional exemplary embodiment of the disclosure, the anti-clusterin antibody or antigen binding fragment thereof is administered once every four weeks.

In accordance with the present disclosure, the anti-clusterin antibody or antigen binding fragment thereof is administered at a dose of between approximately 3 mg/kg and approximately 20 mg/kg.

In some embodiments, the anti-clusterin antibody or antigen binding fragment thereof is administered at a dose of approximately 3.0 mg/kg.

In some embodiments, the anti-clusterin antibody or antigen binding fragment thereof is administered at a dose of approximately 4.0 mg/kg.

In some embodiments, the anti-clusterin antibody or antigen binding fragment thereof is administered at a dose of approximately 5.0 mg/kg.

In some embodiments, the anti-clusterin antibody or antigen binding fragment thereof is administered at a dose of approximately 6.0 mg/kg.

In some embodiments, the anti-clusterin antibody or antigen binding fragment thereof is administered at a dose of approximately 7.0 mg/kg.

In some embodiments, the anti-clusterin antibody or antigen binding fragment thereof is administered at a dose of approximately 8.0 mg/kg.

In some embodiments, the anti-clusterin antibody or antigen binding fragment thereof is administered at a dose of approximately 9.0 mg/kg.

In some embodiments, the anti-clusterin antibody or antigen binding fragment thereof is administered at a dose of approximately 10.0 mg/kg.

In some embodiments, the anti-clusterin antibody or antigen binding fragment thereof is administered at a dose of approximately 11.0 mg/kg.

In some embodiments, the anti-clusterin antibody or antigen binding fragment thereof is administered at a dose of approximately 12.0 mg/kg.

In some embodiments, the anti-clusterin antibody or antigen binding fragment thereof is administered at a dose of approximately 13.0 mg/kg.

In some embodiments, the anti-clusterin antibody or antigen binding fragment thereof is administered at a dose of approximately 14.0 mg/kg.

In some embodiments, the anti-clusterin antibody or antigen binding fragment thereof is administered at a dose of approximately 15.0 mg/kg.

In some embodiments, the anti-clusterin antibody or antigen binding fragment thereof is administered at a dose of approximately 16.0 mg/kg.

In some embodiments, the anti-clusterin antibody or antigen binding fragment thereof is administered at a dose of approximately 17.0 mg/kg.

In some embodiments, the anti-clusterin antibody or antigen binding fragment thereof is administered at a dose of approximately 18.0 mg/kg.

In some embodiments, the anti-clusterin antibody or antigen binding fragment thereof is administered at a dose of approximately 19.0 mg/kg.

In some embodiments, the anti-clusterin antibody or antigen binding fragment thereof 0 is administered at a dose of approximately 20.0 mg/kg.

In accordance with the present disclosure, the anti-clusterin antibody or antigen binding fragment thereof is humanized 16B5 and is administered at a dose of between approximately 3 mg/kg and approximately 20 mg/kg.

In accordance with the present disclosure, humanized 16B5 is administered at a dose of between approximately 4 mg/kg and approximately 20 mg/kg.

In accordance with the present disclosure, the humanized 16B5 is administered at a dose of between approximately 5 mg/kg and approximately 20 mg/kg.

In accordance with the present disclosure, humanized 16B5 is administered at a dose of between approximately 6 mg/kg and approximately 20 mg/kg.

In accordance with the present disclosure, humanized 16B5 is administered at a dose of between approximately 6 mg/kg and approximately 18 mg/kg.

In accordance with the present disclosure, humanized 16B5 is administered at a dose of between approximately 6 mg/kg and approximately 17 mg/kg.

In accordance with the present disclosure, humanized 16B5 is administered at a dose of between approximately 6 mg/kg and approximately 16 mg/kg.

In accordance with the present disclosure, humanized 16B5 administered at a dose of between approximately 6 mg/kg and approximately 15 mg/kg.

In accordance with the present disclosure, humanized 16B5 is administered at a dose of between approximately 6 mg/kg and approximately 14 mg/kg.

In accordance with the present disclosure, humanized 16B5 is administered at a dose of between approximately 6 mg/kg and approximately 13 mg/kg.

In accordance with the present disclosure, humanized 16B5 is administered at a dose of between approximately 6 mg/kg and approximately 12 mg/kg.

In accordance with the present disclosure, humanized 16B5 is administered at a dose of between approximately 7 mg/kg and approximately 12 mg/kg.

In accordance with the present disclosure, humanized 16B5 is administered at a dose of between approximately 8 mg/kg and approximately 12 mg/kg.

In accordance with the present disclosure, humanized 16B5 is administered at a dose of between approximately 9 mg/kg and approximately 12 mg/kg.

In some embodiments, the anti-clusterin antibody or antigen binding fragment thereof is humanized 16B5 and is administered at a dose of approximately 3.0 mg/kg.

In some embodiments, the anti-clusterin antibody or antigen binding fragment thereof is humanized 16B5 and is administered at a dose of approximately 4.0 mg/kg.

In some embodiments, the anti-clusterin antibody or antigen binding fragment thereof is humanized 16B5 and is administered at a dose of approximately 5.0 mg/kg.

In some embodiments, the anti-clusterin antibody or antigen binding fragment thereof is humanized 16B5 and is administered at a dose of approximately 6.0 mg/kg.

In some embodiments, the anti-clusterin antibody or antigen binding fragment thereof is humanized 16B5 and is administered at a dose of approximately 7.0 mg/kg.

In some embodiments, the anti-clusterin antibody or antigen binding fragment thereof is humanized 16B5 and is administered at a dose of approximately 8.0 mg/kg.

In some embodiments, the anti-clusterin antibody or antigen binding fragment thereof is humanized 16B5 and is administered at a dose of approximately 9.0 mg/kg.

In some embodiments, the anti-clusterin antibody or antigen binding fragment thereof is humanized 16B5 and is administered at a dose of approximately 10.0 mg/kg.

In some embodiments, the anti-clusterin antibody or antigen binding fragment thereof is humanized 16B5 and is administered at a dose of approximately 11.0 mg/kg.

In some embodiments, the anti-clusterin antibody or antigen binding fragment thereof is humanized 16B5 and is administered at a dose of approximately 12.0 mg/kg.

In some embodiments, the anti-clusterin antibody or antigen binding fragment thereof is humanized 16B5 and is administered at a dose of approximately 13.0 mg/kg.

In some embodiments, the anti-clusterin antibody or antigen binding fragment thereof is humanized 16B5 and is administered at a dose of approximately 14.0 mg/kg.

In some embodiments, the anti-clusterin antibody or antigen binding fragment thereof is humanized 16B5 and is administered at a dose of approximately 15.0 mg/kg.

In some embodiments, the anti-clusterin antibody or antigen binding fragment thereof is humanized 16B5 and is administered at a dose of approximately 16.0 mg/kg.

In some embodiments, the anti-clusterin antibody or antigen binding fragment thereof is humanized 16B5 and is administered at a dose of approximately 17.0 mg/kg.

In some embodiments, the anti-clusterin antibody or antigen binding fragment thereof is humanized 16B5 and is administered at a dose of approximately 18.0 mg/kg.

In some embodiments, the anti-clusterin antibody or antigen binding fragment thereof is humanized 16B5 and is administered at a dose of approximately 19.0 mg/kg.

In some embodiments, the anti-clusterin antibody or antigen binding fragment thereof is humanized 16B5 and is administered at a dose of approximately 20.0 mg/kg.

### Docetaxel

In accordance with the present disclosure, docetaxel is administered at a dose sufficient to allow chemotherapy-induced immunogenic modulation of tumor.

In some embodiments, the dose of the docetaxel is a therapeutically effective and safe dose.

In accordance with the present disclosure, docetaxel is administered at an administration interval sufficient to allow chemotherapy-induced immunogenic modulation of tumor.

In accordance with the present disclosure, docetaxel is administered for a treatment period sufficient to allow chemotherapy-induced immunogenic modulation of tumor.

In some embodiments, docetaxel is administered at a dose and/or an administration interval and/or for a treatment period sufficient to allow chemotherapy-induced immunogenic modulation of tumor.

In accordance with an exemplary embodiment of the disclosure, docetaxel is administered once every week.

In accordance with another exemplary embodiment of the disclosure, docetaxel is administered once every two weeks.

In accordance with yet another exemplary embodiment of the disclosure, docetaxel is administered once every three weeks.

In accordance with a further exemplary embodiment of the disclosure, docetaxel is administered once every four weeks.

In accordance with a further exemplary embodiment of the disclosure, docetaxel is administered once every five weeks.

In accordance with a further exemplary embodiment of the disclosure, docetaxel is administered once every six weeks.

In accordance with the present disclosure, docetaxel is administered at a dose of between approximately 60 mg/m² to approximately 100 mg/m².

In accordance with the present disclosure docetaxel is administered at a dose of between approximately 60 mg/m² to approximately 95 mg/m².

In accordance with the present disclosure docetaxel is administered at a dose of between approximately 60 mg/m² to approximately 90 mg/m².

In accordance with the present disclosure docetaxel is administered at a dose of between approximately 60 mg/m² to approximately 85 mg/m².

In accordance with the present disclosure docetaxel is administered at a dose of between approximately 60 mg/m² to approximately 80 mg/m².

In accordance with the present disclosure docetaxel is administered at a dose of between approximately 60 mg/m² to approximately 75 mg/m².

In accordance with the present disclosure docetaxel is administered at a dose of between approximately 70 mg/m² to approximately 75 mg/m².

In some embodiments, docetaxel is administered at a dose of approximately 60 mg/m².

In some embodiments, docetaxel is administered at a dose of approximately 65 mg/m².

In some embodiments, docetaxel is administered at a dose of approximately 70 mg/m².

In some embodiments, docetaxel is administered at a dose of approximately 75 mg/m².

In some embodiments, docetaxel is administered at a dose of approximately 80 mg/m².

In some embodiments, docetaxel is administered at a dose of approximately 85 mg/m².

In some embodiments, docetaxel is administered at a dose of approximately 90 mg/m².

In some embodiments, docetaxel is administered at a dose of approximately 95 mg/m².

In some embodiments, docetaxel is administered at a dose of approximately 100 mg/m².

### Combinations

The present disclosure provides in some aspects and embodiments, an anti-cancer therapy comprising an anti-clusterin antibody or antigen binding fragment thereof for allowing infiltration of immune cells in a tumor (e.g., solid tumor) microenvironment in a subject having cancer.

In some embodiments, the anti-cancer therapy comprises an anti-clusterin antibody or antigen binding fragment thereof and an anti-cancer agent.

In some instances, the anti-cancer therapy is a combination therapy.

The combination therapy may thus comprise an anti-clusterin antibody or antigen binding fragment thereof and an anti-cancer agent such as, for example, and without limitations, radiation therapy or chemotherapy.

Accordingly, in some embodiments, the combination therapy comprises an anti-clusterin antibody or antigen binding fragment thereof and radiation therapy.

In other embodiments, the combination therapy comprises an anti-clusterin antibody or antigen binding fragment thereof and chemotherapy.

In exemplary embodiments, the anti-clusterin antibody or antigen binding fragment thereof is used to overcome or lessen chemotherapeutic resistance mediated by MDR1/P-glycoprotein.

Accordingly, the combination therapy may comprise a chemotherapeutic that is associated with resistance mediated by MDR1/P-glycoprotein.

Exemplary embodiments of chemotherapy include an alkylating agent, an anti-metabolite, an alkaloid, an anti-tumor antibiotic or combination thereof.

In some instances, the alkylating agent may be selected, for example, from altretamine, busulfan, carboplatin, carmustine, cisplatin, cyclophosphamide, dacarbazine, ifosfamide, lomustine, melphalan, temozolomide, trabectedin or derivatives or analogs thereof.

In some instances, the anti-metabolite may be selected, for example, 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, trifluridine, tipiracil or derivatives or analogs thereof.

In some instances, the alkaloid may be selected, for example, from vincristine, vinblastine, vinorelbine, taxanes, etoposide, teniposide, irinotecan, topotecan or derivatives or analogs thereof.

Exemplary embodiments of taxane includes docetaxel, paclitaxel and derivatives or analogues including for example and without limitations, Abraxane®, Cabazitaxel, larotaxel, milataxel, ortataxel, tesetaxel and others.

In some instance, the taxane is docetaxel.

In some instance, the taxane is paclitaxel.

In some instances, the anti-tumor antibiotic may be selected, for example, from daunorubicin, doxorubicin, doxorubicin liposomal, epirubicin, idarubicin, valrubicin, derivatives or analogs thereof.

The present disclosure provides in other aspects and embodiments, a combination therapy which comprises a pharmaceutical composition comprising an anti-clusterin antibody or antigen binding fragment thereof formulated for administration at a dose of between approximately 3 mg/kg and approximately 20 mg/kg and a pharmaceutical composition comprising docetaxel formulated for administration at a dose of approximately 60 mg/m² to 100 mg/m².

In some embodiments, the combination therapy is for use in allowing infiltration of immune cells in a tumor (e.g., solid tumor) microenvironment.

In other embodiments, the combination therapy is for use in treating a subject having cancer.

In yet other embodiments, the combination therapy is for use in treating a subject having cancer and a functional immune system.

In yet other embodiments, the combination therapy is for use in treating a subject having cancer and an adequate organ and immune function.

In accordance with the present disclosure, the anti-clusterin antibody or antigen binding fragment thereof and docetaxel are each administered at a dose a sufficient to allow infiltration of immune cells in a tumor microenvironment and/or chemotherapy-induced immunogenic modulation of tumor.

In accordance with the present disclosure, the anti-clusterin antibody or antigen binding fragment thereof and docetaxel are each administered at administration interval sufficient to allow infiltration of immune cells in a tumor microenvironment and/or chemotherapy-induced immunogenic modulation of tumor.

In accordance with the present disclosure, the anti-clusterin antibody or antigen binding fragment thereof and docetaxel are each administered for a treatment period sufficient to allow infiltration of immune cells in a tumor microenvironment and/or chemotherapy-induced immunogenic modulation of tumor.

In accordance with the present disclosure, the anti-clusterin antibody or antigen binding fragment thereof and docetaxel are each administered at a dose, administration interval and/or for a treatment period sufficient to allow infiltration of immune cells in a tumor microenvironment and/or chemotherapy-induced immunogenic modulation of tumor.

In some embodiments, the anti-clusterin antibody or antigen binding fragment thereof is administered at a dose of approximately 12 mg/kg once weekly, and docetaxel is administered at a dose of approximately 75 mg/m² once every three weeks.

In some embodiments, the anti-clusterin antibody or antigen binding fragment thereof is administered at a dose of approximately 12 mg/kg once weekly, and docetaxel is administered at a dose of approximately 60 mg/m² once every three weeks.

In some embodiments, the anti-clusterin antibody or antigen binding fragment thereof is administered at a dose of approximately 9 mg/kg once weekly, and docetaxel is administered at a dose of approximately 75 mg/m² once every three weeks.

In some embodiments, the anti-clusterin antibody or antigen binding fragment thereof is administered at a dose of approximately 9 mg/kg once weekly, and docetaxel is administered at a dose of approximately 60 mg/m² once every three weeks.

In some embodiments, the anti-clusterin antibody or antigen binding fragment thereof is administered at a dose of approximately 6 mg/kg once weekly, and docetaxel is administered at a dose of approximately 75 mg/m² once every three weeks.

In some embodiments, the anti-clusterin antibody or antigen binding fragment thereof is administered at a dose of approximately 6 mg/kg once weekly, and docetaxel is administered at a dose of approximately 60 mg/m² once every three weeks.

In some embodiments, the anti-clusterin antibody or antigen binding fragment thereof is administered at a dose of approximately 3 mg/kg once weekly, and docetaxel is administered at a dose of approximately 75 mg/m² once every three weeks.

In some embodiments, the anti-clusterin antibody or antigen binding fragment thereof is administered at a dose of approximately 3 mg/kg once weekly, and docetaxel is administered at a dose of approximately 60 mg/m² once every three weeks.

The anti-clusterin antibody or antigen binding fragment thereof used in the combination therapy is as described herein.

For example, the anti-clusterin antibody or antigen binding fragment thereof that is used in the combination therapy may have a light chain variable region comprising the complementarity determining regions (CDRs) of the light chain variable region set forth in SEQ ID NO:9 and a heavy chain variable region comprising the CDRs of the heavy chain variable region set forth in SEQ ID NO:10.

In an exemplary embodiment, the anti-clusterin antibody or antigen binding fragment thereof that is used in the combination therapy may have a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3 and a heavy chain variable region comprising a CDRH1 having the amino acid sequence set forth in SEQ ID NO:4, a CDRH2 having the amino acid sequence set forth in SEQ ID NO:5, a CDRH3 having the amino acid sequence set forth in SEQ ID NO:6.

In another exemplary embodiment, the anti-clusterin antibody or antigen binding fragment thereof that is used in the combination therapy may have a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3 and a heavy chain variable region comprising a CDRH1 having the amino acid sequence set forth in SEQ ID NO:35, a CDRH2 having the amino acid sequence set forth in SEQ ID NO:36, a CDRH3 having the amino acid sequence set forth in SEQ ID NO:37.

In yet another exemplary embodiment, the anti-clusterin antibody or antigen binding fragment thereof that is used in the combination therapy may have a light chain variable region having an amino acid sequence having at least 80%, at least 85%, at least 90%, at least 95%, at least 99% identity with the amino acid sequence set forth in SEQ ID NO:7 and a heavy chain variable region having an amino acid sequence at least 80%, at least 85%, at least 90%, at least 95%, at least 99% identity with the amino acid sequence set forth in SEQ ID NO:8.

In some embodiments, the anti-clusterin antibody or antigen binding fragment thereof comprises a light chain variable region having an amino acid sequence identical to the amino acid sequence set forth in SEQ ID NO:7 and a heavy chain variable region having an amino acid sequence identical to the amino acid sequence set forth in SEQ ID NO:8.

In some embodiments, the anti-clusterin antibody or antigen binding fragment thereof comprises a light chain variable region having an amino acid sequence having at least 80%, at least 85%, at least 90%, at least 95%, at least 99% identity with the amino acid sequence set forth in SEQ ID NO:9 and a heavy chain variable region having an amino acid sequence at least 80%, at least 85%, at least 90%, at least 95%, at least 99% identity with the amino acid sequence set forth in SEQ ID NO:10.

In some embodiments, the anti-clusterin antibody or antigen binding fragment thereof comprises a light chain variable region having an amino acid sequence identical to the amino acid sequence set forth in SEQ ID NO:9 and a heavy chain variable region having an amino acid sequence identical to the amino acid sequence set forth in SEQ ID NO: 10.

In some embodiments, the anti-clusterin antibody or antigen binding fragment thereof comprises a light chain having an amino acid sequence having at least 80%, at least 85%, at least 90%, at least 95%, at least 99% identity with the amino acid sequence set forth in SEQ ID NO:11 and a heavy chain having an amino acid sequence having at least 80%, at least 85%, at least 90%, at least 95%, at least 99% identity with the amino acid sequence set forth in SEQ ID NO:12.

In some embodiments, the anti-clusterin antibody or antigen binding fragment thereof comprises a light chain having an amino acid sequence identical the amino acid sequence set forth in SEQ ID NO:11 and a heavy chain having an amino acid sequence identical to the amino acid sequence set forth in SEQ ID NO:12.

In some embodiments, the anti-clusterin antibody or antigen binding fragment thereof is humanized 16B5 and is administered at a dose of 12 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 12 mg/kg once weekly, and docetaxel is administered at a dose of 60 mg/m² once every three weeks.

In some embodiments, the anti-clusterin antibody or antigen binding fragment thereof is humanized 16B5 and is administered at a dose of 9 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 9 mg/kg once weekly, and docetaxel is administered at a dose of 60 mg/m² once every three weeks.

In some embodiments, the anti-clusterin antibody or antigen binding fragment thereof is humanized 16B5 and is administered at a dose of 6 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 6 mg/kg once weekly, and docetaxel is administered at a dose of 60 mg/m² once every three weeks.

In some embodiments, the anti-clusterin antibody or antigen binding fragment thereof is humanized 16B5 and is administered at a dose of 3 mg/kg once weekly, and docetaxel is administered at a dose of 75 mg/m² once every three weeks.

In some embodiments, the anti-clusterin antibody or antigen binding fragment thereof is humanized 16B5 and is administered at a dose of 3 mg/kg once weekly, and docetaxel is administered at a dose of 60 mg/m² once every three weeks.

In some embodiments, the anti-clusterin antibody or antigen binding fragment thereof is humanized 16B5 and is administered at a dose of approximately 3 mg/kg to approximately 20 mg/kg once weekly, and docetaxel is administered at a dose of between approximately 60 mg/m² to approximately 100 mg/m² once every three weeks and both are essentially administered over the entire course of the treatment period.

In some embodiments, the anti-clusterin antibody or antigen binding fragment thereof is humanized 16B5 and is administered at a dose of approximately 4 mg/kg to approximately 18 mg/kg once weekly, and docetaxel is administered at a dose of between approximately 60 mg/m² to approximately 100 mg/m² once every three weeks and both are essentially administered over the entire course of the treatment period.

In some embodiments, the anti-clusterin antibody or antigen binding fragment thereof is humanized 16B5 and is administered at a dose of approximately 5 mg/kg to approximately 16 mg/kg once weekly, and docetaxel is administered at a dose of between approximately 60 mg/m² to approximately 100 mg/m² once every three weeks and both are essentially administered over the entire course of the treatment period.

In some embodiments, the anti-clusterin antibody or antigen binding fragment thereof is humanized 16B5 and is administered at a dose of approximately 6 mg/kg to approximately 15 mg/kg once weekly, and docetaxel is administered at a dose of between approximately 60 mg/m² to approximately 100 mg/m² once every three weeks and both are essentially administered over the entire course of the treatment period.

In some embodiments, the anti-clusterin antibody or antigen binding fragment thereof is humanized 16B5 and is administered at a dose of approximately 6 mg/kg to approximately 12 mg/kg once weekly, and docetaxel is administered at a dose of between approximately 60 mg/m² to approximately 100 mg/m² once every three weeks and both are essentially administered over the entire course of the treatment period.

In some embodiments, the anti-clusterin antibody or antigen binding fragment thereof is humanized 16B5 and is administered at a dose of 12 mg/kg once weekly, and docetaxel is administered at a dose of 60 mg/m² once every three weeks and both are essentially administered over the entire course of the treatment period.

In some embodiments, the anti-clusterin antibody or antigen binding fragment thereof is humanized 16B5 and is administered at a dose of 9 mg/kg once weekly, and docetaxel is administered at a dose of 75 mg/m² once every three weeks and both are essentially administered over the entire course of the treatment period.

In some embodiments, the anti-clusterin antibody or antigen binding fragment thereof is humanized 16B5 and is administered at a dose of 9 mg/kg once weekly, and docetaxel is administered at a dose of 60 mg/m² once every three weeks and both are essentially administered over the entire course of the treatment period.

In some embodiments, the anti-clusterin antibody or antigen binding fragment thereof is humanized 16B5 and is administered at a dose of 6 mg/kg once weekly, and docetaxel is administered at a dose of 75 mg/m² once every three weeks and both are essentially administered over the entire course of the treatment period.

In some embodiments, the anti-clusterin antibody or antigen binding fragment thereof is humanized 16B5 and is administered at a dose of 6 mg/kg once weekly, and docetaxel is administered at a dose of 60 mg/m² once every three weeks and both are essentially administered over the entire course of the treatment period.

In some embodiments, the anti-clusterin antibody or antigen binding fragment thereof is humanized 16B5 and is administered at a dose of 3 mg/kg once weekly, and docetaxel is administered at a dose of 75 mg/m² once every three weeks and both are essentially administered over the entire course of the treatment period.

In some embodiments, the anti-clusterin antibody or antigen binding fragment thereof is humanized 16B5 and is administered at a dose of 3 mg/kg once weekly, and docetaxel is administered at a dose of 60 mg/m² once every three weeks and both are essentially administered over the entire course of the treatment period.

In some embodiments, a treatment cycle is considered completed after a period of approximately seven days after a subject has received both the anti-clusterin antibody or antigen binding fragment thereof and docetaxel.

For example, when both the anti-clusterin antibody or antigen binding fragment thereof and docetaxel are administered every week, a treatment cycle is considered to be of 7 days.

For example, when the anti-clusterin antibody or antigen binding fragment thereof is administered every week and docetaxel is administered every two weeks, a treatment cycle is considered to of 14 days.

For example, when the anti-clusterin antibody or antigen binding fragment thereof is administered every week and docetaxel is administered every three weeks, a treatment cycle is considered to of 21 days.

In some exemplary embodiments, one treatment cycle is approximately 21 days.

In some exemplary embodiments, essentially all treatment cycles are approximately 21 days.

In some exemplary embodiments, each treatment cycles are approximately 21 days.

In accordance with the present disclosure, the subject may thus receive a new treatment cycle every 21 days.

In accordance with the present disclosure, a subject may receive at least one treatment cycle.

In accordance with the present disclosure, a subject may receive at least two treatment cycles.

In accordance with the present disclosure, a subject may receive at least three treatment cycles.

In accordance with the present disclosure, a subject may receive at least four treatment cycles.

In accordance with the present disclosure, a subject may receive four or more treatment cycles.

In accordance with the present disclosure, a subject may receive at least five treatment cycles.

In accordance with the present disclosure, a subject may receive at least six treatment cycles.

In accordance with the present disclosure, a subject may receive at least seven treatment cycles.

In accordance with the present disclosure, a subject may receive at least eight treatment cycles.

In accordance with the present disclosure, a subject may receive at least nine treatment cycles.

In accordance with the present disclosure, a subject may receive at least ten treatment cycles.

In accordance with the present disclosure, a subject may receive at least eleven treatment cycles.

In accordance with the present disclosure, a subject may receive at least twelve treatment cycles.

In accordance with the present disclosure, a subject may receive at least thirteen treatment cycles.

In accordance with the present disclosure, a subject may receive at least fourteen treatment cycles.

In accordance with the present disclosure, a subject may receive at least fifteen treatment cycles.

In accordance with the present disclosure, a subject may receive at least sixteen treatment cycles.

In accordance with the present disclosure, a subject may receive at least seventeen treatment cycles.

In accordance with the present disclosure, a subject may receive at least eighteen treatment cycles.

In accordance with the present disclosure, a subject may receive at least nineteen treatment cycles.

In accordance with the present disclosure, a subject may receive at least twenty treatment cycles.

In accordance with the present disclosure, a subject may receive more than twenty treatment cycles.

In some embodiments, the anti-clusterin antibody or antigen binding fragment thereof is administered by infusion, such as for example, an intravenous infusion.

In some embodiments, the anti-clusterin antibody or antigen binding fragment thereof is administered by infusion over approximately a 1-hour time frame.

In some embodiments, docetaxel is administered by infusion over approximately a 1-hour time frame.

In accordance with the present disclosure, the anti-clusterin antibody or antigen binding fragment thereof and docetaxel are administered on same day.

The anti-clusterin antibody or antigen binding fragment thereof and docetaxel may be administered separately.

The anti-clusterin antibody or antigen binding fragment thereof and docetaxel may be administered sequentially.

In some embodiments, the anti-clusterin antibody or antigen binding fragment thereof is administered by infusion over approximately a 1-hour time frame and docetaxel is subsequently administered by infusion on same day over approximately a 1-hour time frame.

In some embodiments, docetaxel is administered by infusion over approximately a 1-hour time frame and the anti-clusterin antibody or antigen binding fragment thereof is subsequently administered by infusion on same day over approximately a 1-hour time frame.

In accordance with the present disclosure, the combination therapy may be used for subjects having carcinoma.

In accordance with the present disclosure, the combination therapy may be used for subjects having metastatic carcinoma.

In some embodiment, the combination therapy may be used for subjects having non-small cell lung cancer (NSCLC) such as metastatic NSCLC or stage III to IV NSCLC.

In exemplary embodiments, the combination therapy may be used for subjects having breast cancer, prostate cancer, gastric cancer, head and neck cancer, thyroid cancer or ovarian cancer.

In exemplary embodiments, the combination therapy may be used for subjects having metastatic breast cancer, metastatic prostate cancer, metastatic gastric cancer, metastatic head and neck cancer, metastatic thyroid cancer or metastatic ovarian cancer.

In accordance with the present disclosure, the pharmaceutical composition comprising the anti-clusterin antibody or antigen binding fragment thereof and the pharmaceutical composition comprising docetaxel are both administered essentially over the entire course of the treatment period.

### Anti-Clusterin Antibodies or Antigen Binding Fragments Thereof

The present disclosure relates to the use of an anti-clusterin antibody or antigen binding fragment thereof either alone (single agent) or in combination with a chemotherapeutic that induces immunogenic modulation such as docetaxel.

In some embodiments, the anti-clusterin antibody or antigen binding fragment thereof of the present disclosure is capable of inhibiting epithelial to mesenchymal transition.

In some embodiments, the anti-clusterin antibody or antigen binding fragment thereof of the present disclosure is capable of binding to amino acids 421 and 443 of a C-terminal portion of a ß-subunit of human clusterin (SEQ ID NO: 41 see PCT/CA2006/001505 published under No. WO2007/030930 and international application No. PCT/CA2010/0001882 published under No. WO2011/063523 the entire content of which is incorporated herein by reference).

In some embodiments, the anti-clusterin antibody or antigen binding fragment thereof of the present disclosure is capable of binding to an epitope comprised within amino acids 421 and 443 of a C-terminal portion of a ß-subunit of human clusterin (SEQ ID NO: 41 see PCT/CA2006/001505 published under No. WO2007/030930 and international application No. PCT/CA2010/0001882 published under No. WO2011/063523 the entire content of which is incorporated herein by reference).

In some embodiments, the anti-clusterin antibody or antigen binding fragment thereof comprises the CDRs of an anti-clusterin antibody or antigen binding fragment thereof of the present disclosure.

In some embodiments, the anti-clusterin antibody or antigen binding fragment thereof is an antibody or antigen binding fragment thereof that is capable of competing with an anti-clusterin antibody or antigen binding fragment thereof of the present disclosure for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the CDRs are identified using methods known to a person skilled in the art and which are reviewed in Antibody Engineering Vol. 2, Chapter 3 by Andrew C. R. Martin, the entire content of which is incorporated herein by reference.

In particular embodiments, all CDRs are identified using the Kabat definition which is the most commonly used definition (Wu and Kabat, 1970).

In particular embodiments, all CDRs are identified using the contact definition (MacCallum et al., 1996) which is likely to be the most useful for people wishing to perform mutagenesis to modify the affinity of an antibody since these are residues which take part in interactions with antigen.

In particular embodiments, the anti-clusterin antibody or antigen binding fragment thereof comprises a light chain variable region comprising the complementarity determining regions (CDRs) of the light chain variable region set forth in SEQ ID NO:9 and a heavy chain variable region comprising the CDRs of the heavy chain variable region set forth in SEQ ID NO:10.

In some exemplary embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3.

In some exemplary embodiments, the anti-clusterin antibody or antigen binding fragment thereof comprises a heavy chain variable region comprising a CDRH1 having the amino acid sequence set forth in SEQ ID NO:4, a CDRH2 having the amino acid sequence set forth in SEQ ID NO:5, a CDRH3 having the amino acid sequence set forth in SEQ ID NO:6.

In some exemplary embodiments, the anti-clusterin antibody or antigen binding fragment thereof comprises a heavy chain variable region comprising a CDRH1 having the amino acid sequence set forth in SEQ ID NO:35, a CDRH2 having the amino acid sequence set forth in SEQ ID NO:36, a CDRH3 having the amino acid sequence set forth in SEQ ID NO:37.

In some embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3 and a heavy chain variable region comprising a CDRH1 having the amino acid sequence set forth in SEQ ID NO:4, a CDRH2 having the amino acid sequence set forth in SEQ ID NO:5, a CDRH3 having the amino acid sequence set forth in SEQ ID NO:6.

In some embodiments, the anti-clusterin antibody or antigen binding fragment thereof comprises a light chain variable region comprising a CDRL1 having the amino acid sequence set forth in SEQ ID NO:1, a CDRL2 having the amino acid sequence set forth in SEQ ID NO:2, a CDRL3 having the amino acid sequence set forth in SEQ ID NO:3 and a heavy chain variable region comprising a CDRH1 having the amino acid sequence set forth in SEQ ID NO:35, a CDRH2 having the amino acid sequence set forth in SEQ ID NO:36, a CDRH3 having the amino acid sequence set forth in SEQ ID NO:37.

In some embodiments, the anti-clusterin antibody or antigen binding fragment thereof comprises a light chain variable region having an amino acid sequence having at least 80% identity with the amino acid sequence set forth in SEQ ID NO:7 and a heavy chain variable region having an amino acid sequence at least 80% identity with the amino acid sequence set forth in SEQ ID NO:8.

In some embodiments, the anti-clusterin antibody or antigen binding fragment thereof comprises a light chain variable region having an amino acid sequence having at least 90% identity with the amino acid sequence set forth in SEQ ID NO:7 and a heavy chain variable region having an amino acid sequence at least 90% identity with the amino acid sequence set forth in SEQ ID NO:8.

In some embodiments, the anti-clusterin antibody or antigen binding fragment thereof comprises a light chain variable region having an amino acid sequence identical to the amino acid sequence set forth in SEQ ID NO:7 and a heavy chain variable region having an amino acid sequence identical to the amino acid sequence set forth in SEQ ID NO:8.

In some embodiments, the anti-clusterin antibody or antigen binding fragment thereof is capable of competing with an antibody comprising a light chain variable region having the amino acid sequence set forth in SEQ ID NO:7 and a heavy chain variable region having the amino acid sequence set forth in SEQ ID NO:8 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain variable region having an amino acid sequence having at least 80% identity with the amino acid sequence set forth in SEQ ID NO:9 and a heavy chain variable region having an amino acid sequence at least 80% identity with the amino acid sequence set forth in SEQ ID NO: 10.

In some embodiments, the anti-clusterin antibody or antigen binding fragment thereof comprises a light chain variable region having an amino acid sequence having at least 90% identity with the amino acid sequence set forth in SEQ ID NO:9 and a heavy chain variable region having an amino acid sequence at least 90% identity with the amino acid sequence set forth in SEQ ID NO: 10.

In some embodiments, the anti-clusterin antibody or antigen binding fragment thereof comprises a light chain variable region having an amino acid sequence identical to the amino acid sequence set forth in SEQ ID NO:9 and a heavy chain variable region having an amino acid sequence identical to the amino acid sequence set forth in SEQ ID NO:10.

In some embodiments, the anti-clusterin antibody or antigen binding fragment thereof is capable of competing with an antibody comprising a light chain variable region having the amino acid sequence set forth in SEQ ID NO:9 and a heavy chain variable region having the amino acid sequence set forth in SEQ ID NO:10 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain having an amino acid sequence having at least 80% identity with the amino acid sequence set forth in SEQ ID NO:11 and a heavy chain having an amino acid sequence having at least 80% identity with the amino acid sequence set forth in SEQ ID NO:12.

In some embodiments, the anti-clusterin antibody or antigen binding fragment thereof comprises a light chain having an amino acid sequence having at least 90% identity with the amino acid sequence set forth in SEQ ID NO:11 and a heavy chain having an amino acid sequence having at least 90% identity with the amino acid sequence set forth in SEQ ID NO:12.

In some embodiments, the anti-clusterin antibody or antigen binding fragment thereof comprises a light chain having an amino acid sequence identical the amino acid sequence set forth in SEQ ID NO:11 and a heavy chain having an amino acid sequence identical to the amino acid sequence set forth in SEQ ID NO: 12.

In some embodiments, the anti-clusterin antibody or antigen binding fragment thereof is capable of competing with an antibody comprising a light chain having the amino acid sequence set forth in SEQ ID NO:11 and a heavy chain having the amino acid sequence set forth in SEQ ID NO: 12 for the binding of clusterin (e.g., secreted clusterin (sCLU) or tumor-associated sCLU (TA-sCLU)) or for binding to a polypeptide comprising the amino acid sequence set forth in SEQ ID NO:41.

In other particular embodiments, the anti-clusterin antibody or antigen binding fragment thereof comprises a light chain variable region comprising a CDRL1 having the amino acid sequence set forth in SEQ ID NO:15, a CDRL2 having the amino acid sequence set forth in SEQ ID NO:16, a CDRL3 having the amino acid sequence set forth in SEQ ID NO:17.

In some exemplary embodiments, the anti-clusterin antibody or antigen binding fragment thereof comprises a heavy chain variable region comprising a CDRH1 having the amino acid sequence set forth in SEQ ID NO:18, a CDRH2 having the amino acid sequence set forth in SEQ ID NO:19, a CDRH3 having the amino acid sequence set forth in SEQ ID NO:20.

In some exemplary embodiments, the anti-clusterin antibody or antigen binding fragment thereof comprises a heavy chain variable region comprising a CDRH1 having the amino acid sequence set forth in SEQ ID NO:38, a CDRH2 having the amino acid sequence set forth in SEQ ID NO:39, a CDRH3 having the amino acid sequence set forth in SEQ ID NO:40.

In some embodiments, the anti-clusterin antibody or antigen binding fragment thereof comprises a light chain variable region comprising a CDRL1 having the amino acid sequence set forth in SEQ ID NO:15, a CDRL2 having the amino acid sequence set forth in SEQ ID NO:16, a CDRL3 having the amino acid sequence set forth in SEQ ID NO: 17 and a heavy chain variable region comprising a CDRH1 having the amino acid sequence set forth in SEQ ID NO:18, a CDRH2 having the amino acid sequence set forth in SEQ ID NO:19, a CDRH3 having the amino acid sequence set forth in SEQ ID NO:20.

In some embodiments, the anti-clusterin antibody or antigen binding fragment thereof comprises a light chain variable region comprising a CDRL1 having the amino acid sequence set forth in SEQ ID NO:15, a CDRL2 having the amino acid sequence set forth in SEQ ID NO:16, a CDRL3 having the amino acid sequence set forth in SEQ ID NO: 17 and a heavy chain variable region comprising a CDRH1 having the amino acid sequence set forth in SEQ ID NO:38, a CDRH2 having the amino acid sequence set forth in SEQ ID NO:39, a CDRH3 having the amino acid sequence set forth in SEQ ID NO:40.

In some embodiments, the anti-clusterin antibody or antigen binding fragment thereof comprises a light chain variable region having an amino acid sequence having at least 80% identity with the amino acid sequence set forth in SEQ ID NO:21 and a heavy chain variable region having an amino acid sequence at least 80% identity with the amino acid sequence set forth in SEQ ID NO:22.

In some embodiments, the anti-clusterin antibody or antigen binding fragment thereof comprises a light chain variable region having an amino acid sequence having at least 90% identity with the amino acid sequence set forth in SEQ ID NO:21 and a heavy chain variable region having an amino acid sequence at least 90% identity with the amino acid sequence set forth in SEQ ID NO:22.

In some embodiments, the anti-clusterin antibody or antigen binding fragment thereof comprises a light chain variable region having an amino acid sequence identical to the amino acid sequence set forth in SEQ ID NO:21 and a heavy chain variable region having an amino acid sequence identical to the amino acid sequence set forth in SEQ ID NO:22.

In some embodiments, the anti-clusterin antibody or antigen binding fragment thereof is capable of competing with an antibody comprising a light chain variable region having the amino acid sequence set forth in SEQ ID NO:21 and a heavy chain variable region having the amino acid sequence set forth in SEQ ID NO:22 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain variable region having an amino acid sequence having at least 80% identity with the amino acid sequence set forth in SEQ ID NO:23 and a heavy chain variable region having an amino acid sequence at least 80% identity with the amino acid sequence set forth in SEQ ID NO:24.

In some embodiments, the anti-clusterin antibody or antigen binding fragment thereof comprises a light chain variable region having an amino acid sequence having at least 90% identity with the amino acid sequence set forth in SEQ ID NO:23 and a heavy chain variable region having an amino acid sequence at least 90% identity with the amino acid sequence set forth in SEQ ID NO:24.

In some embodiments, the anti-clusterin antibody or antigen binding fragment thereof comprises a light chain variable region having an amino acid sequence identical to the amino acid sequence set forth in SEQ ID NO:23 and a heavy chain variable region having an amino acid sequence identical to the amino acid sequence set forth in SEQ ID NO:24.

In some embodiments, the anti-clusterin antibody or antigen binding fragment thereof is capable of competing with an antibody comprising a light chain variable region having the amino acid sequence set forth in SEQ ID NO:23 and a heavy chain variable region having the amino acid sequence set forth in SEQ ID NO:24 for the binding of clusterin (e.g., secreted clusterin (sCLU) or tumor-associated sCLU (TA-sCLU)) or for binding to a polypeptide comprising the amino acid sequence set forth in SEQ ID NO:41.

In some embodiments, the anti-clusterin antibody or antigen binding fragment thereof comprises a light chain having an amino acid sequence having at least 80% identity with the amino acid sequence set forth in SEQ ID NO:25 and a heavy chain having an amino acid sequence having at least 80% identity with the amino acid sequence set forth in SEQ ID NO:26.

In some embodiments, the anti-clusterin antibody or antigen binding fragment thereof comprises a light chain having an amino acid sequence having at least 90% identity with the amino acid sequence set forth in SEQ ID NO:25 and a heavy chain having an amino acid sequence having at least 90% identity with the amino acid sequence set forth in SEQ ID NO:26.

In some embodiments, the anti-clusterin antibody or antigen binding fragment thereof comprises a light chain having an amino acid sequence identical the amino acid sequence set forth in SEQ ID NO:25 and a heavy chain having an amino acid sequence identical to the amino acid sequence set forth in SEQ ID NO:26.

In some embodiments, the anti-clusterin antibody or antigen binding fragment thereof is capable of competing with an antibody comprising a light chain having the amino acid sequence set forth in SEQ ID NO:25 and a heavy chain having the amino acid sequence set forth in SEQ ID NO:26 for the binding of clusterin (e.g., secreted clusterin (sCLU) or tumor-associated sCLU (TA-sCLU)) or for binding to a polypeptide comprising the amino acid sequence set forth in SEQ ID NO:41.

In yet other particular embodiments, the anti-clusterin antibody or antigen binding fragment thereof comprises the CDRs, variable regions or full chains amino acid sequence of the antibody or antigen binding fragment thereof listed in Table 9. The amino acid sequence of antibodies identified as 16B5, 21B12, 20E11, 11E2 and 16C11 is disclosed in international application No. PCT/CA2006/001505 filed on Sep. 13, 2006 and published on Mar. 22, 2007 under no. WO2007/030930 the entire content of which is incorporated herein by reference. The amino acid sequence of murine 16B5, humanized 16B5, murine 21B12 and humanized 21B12 is disclosed in international application No. PCT/CA2010/001882 filed on Nov. 24, 2010 and published on Jun. 3, 2011 under No. WO2011/063523, the entire content of which is incorporated herein by reference.

In yet further particular embodiments, the anti-clusterin antibody or antigen binding fragment thereof may be able to compete with one or more of the antibody or antigen binding fragment thereof listed in Table 9.

### Subject

The anti-cancer therapy disclosed herein is administered to a subject such as a subject in need. In some embodiments, the subject in need is a subject having cancer.

In accordance with the present disclosure, the subject is a human.

In some embodiments, the subject is an adult of 18 years of age or older.

The single and combination therapy disclosed herein is generally administered to a human subject.

In some aspects and embodiments of the present disclosure, the subject in need is a subject having cancer.

In some aspects and embodiments of the present disclosure, the subject in need is a subject having or selected for having an early-stage cancer.

In some aspects and embodiments of the present disclosure, the subject in need is a subject having or selected for having a late-stage cancer.

In some aspects and embodiments of the present disclosure, the subject in need is a subject having or selected for having a resectable tumor.

In other aspects and embodiments of the present disclosure, the subject in need is a subject having cancer and having a functional immune system.

Accordingly, the subject has or is selected for having a functional immune system.

In yet other aspects and embodiments of the present disclosure, the subject in need is a subject having cancer and adequate organ and immune function.

Accordingly, the subject has or is selected for having an adequate organ and immune function.

In some exemplary embodiments, the subject does not receive concurrent anti-cancer treatment with the anti-clusterin antibody or antigen binding fragment thereof single agent.

In some exemplary embodiments, the subject does not receive concurrent anti-cancer treatment with the anti-clusterin antibody or antigen binding fragment thereof and docetaxel combination therapy.

In some exemplary embodiments, the subject does not require concurrent anti-cancer treatment with the anti-clusterin antibody or antigen binding fragment thereof single agent.

In some exemplary embodiments, the subject does not require concurrent anti-cancer treatment with the anti-clusterin antibody or antigen binding fragment thereof and docetaxel combination therapy.

In some embodiments, the subject in need has or is selected for having a carcinoma.

In some embodiments, the subject in need has or is selected for having an early-stage carcinoma.

In some embodiments, the subject in need has or is selected for having a late-stage carcinoma.

In some embodiments, the subject in need has or is selected for having a metastatic carcinoma.

In some embodiments, the subject in need has or is selected for having non-small cell lung cancer (NSCLC).

In some embodiments, the subject in need has or is selected for having metastatic NSCLC.

In some embodiments, the subject in need has or is selected for having stage III to IV NSCLC.

In some embodiments, the subject in need has or is selected for having stage III NSCLC.

In some embodiments, the subject in need has or is selected for having stage IV NSCLC.

In some embodiments, the subject has or is selected for having lung adenocarcinoma.

In some embodiments, the subject has or is selected for having squamous cell lung cancer.

In some embodiments, the subject has or is selected for having large cell lung carcinoma.

In some embodiments, the subject has or is selected for having adenosquamous lung carcinoma.

In some embodiments, the subject has or is selected for having non-small cell lung cancer characterized has having a KRAS mutation.

In some embodiments, the subject in need has or is selected for having breast cancer.

In some embodiments, the subject in need has or is selected for having metastatic breast cancer.

In some embodiments, the subject in need has or is selected for having prostate cancer.

In some embodiments, the subject in need has or is selected for having metastatic prostate cancer.

In some embodiments, the subject in need has or is selected for having bladder cancer.

In some embodiments, the subject in need has or is selected for having metastatic bladder cancer.

In some embodiments, the subject in need has or is selected for having cervical cancer.

In some embodiments, the subject in need has or is selected for having metastatic cervical cancer.

In some embodiments, the subject in need has or is selected for having gastric cancer.

In some embodiments, the subject in need has or is selected for having metastatic gastric cancer.

In some embodiments, the subject in need has or is selected for having head and neck cancer.

In some embodiments, the subject in need has or is selected for having metastatic head and neck cancer.

In some embodiments, the subject in need has or is selected for having thyroid cancer.

In some embodiments, the subject in need has or is selected for having metastatic thyroid cancer.

In some embodiments, the subject in need has or is selected for having ovarian cancer.

In some embodiments, the subject in need has or is selected for having metastatic ovarian cancer.

In some embodiments, the subject in need has or is selected for having endometrial cancer.

In some embodiments, the subject in need has or is selected for having metastatic endometrial cancer.

In some embodiments, the subject in need has or is selected for having liver cancer.

In some embodiments, the subject in need has or is selected for having metastatic liver cancer.

In some embodiments, the subject in need has or is selected for having colorectal cancer.

In some embodiments, the subject in need has or is selected for having metastatic colorectal cancer.

In some embodiments, the subject in need has or is selected for having pancreatic cancer.

In some embodiments, the subject in need has or is selected for having metastatic pancreatic cancer.

In some embodiments, the subject in need has or is selected for having cholangiocarcinoma.

In some embodiments, the subject in need has or is selected for having metastatic cholangiocarcinoma.

In some embodiments, the subject in need has or is selected for having mesothelioma.

In some embodiments, the subject in need has or is selected for having metastatic mesothelioma.

In some embodiments, the subject in need has or is selected for having melanoma.

In some embodiments, the subject in need has or is selected for having metastatic melanoma.

In some embodiments, the subject in need has or is selected for having a tumor characterized as immunologically cold.

In some embodiments, the subject in need has or is selected for having one or more lesions that are immunologically cold.

In some embodiments, the subject has or is selected for having one or more lesions with poor infiltration of immune cells. In some instances, the level of immune cell infiltration may be determined by a trained pathologist. In other instances, the level of immune cell infiltration may be determined, for example, by computer-based quantification of tumor imaging. Other techniques may be used.

In some embodiments, the subject in need has or is selected for having a tumor characterized as immunologically warm or hot that is non-responsive to immunotherapy.

In some embodiments, the subject has or is selected for having one or more lesions having an EMT signature or showing signs of an EMT signature. The EMT signature may be determined by a trained pathologist. The EMT signature may be determined, for example, by computer-based quantification of tumor imaging.

In some embodiments, the EMT signature may be assessed by one or more EMT biomarkers. For example, an EMT signature may be associated with the acquisition of mesenchymal markers and/or with attenuation of epithelial markers especially associated with EMT type 3 (see Zeisber, M. and E. G. Neilson, The Journal of Clin Investig, 119:1429-1437 (2009)).

EMT biomarkers include for example, cell-surface proteins such as, for example, N-cadherin, OB-cadherin, α5β1 integrin, αVβ6 integrin, syndecan-1, cytoskeletal markers such as, for example, FSP1, α-SMA, vimentin, B-catenin, α1(I) collagen, α1(III) collagen, fibronectin, laminin 5, transcription factors such as, for example, snail1, snail2, ZEB1, CBF-A/KAP-1 complex, twist, LEF-1, Ets-1. FOXC2, goosecoid, and mircroRNAs such as, for example, miR10b, miR-21 (acquired markers), E-cadherin, ZO-1, cytokeratin, α1(IV) collagen, laminin-1 (attenuated markers) (see Zeisber, M. and E. G. Neilson, The Journal of Clin Investig, 119:1429-1437 (2009)).

In some exemplary embodiments, the subject is a subject that has not received prior treatment comprising a PD-1 or PD-L1 immune checkpoint inhibitor.

In some exemplary embodiments, the subject is a subject that has not received prior treatment comprising an anti-PD-1 or anti-PD-L1 immune checkpoint antibody.

In some exemplary embodiments, the subject is a subject that has received prior treatment comprising a PD-1 or PD-L1 immune checkpoint inhibitor.

In other exemplary embodiments, the subject is a subject that has received or is selected for having received prior treatment comprising an anti-PD-1 or anti-PD-L1 immune checkpoint antibody.

In further embodiments, the subject has received or is selected for having received prior treatment comprising an anti-PD-1 or anti-PD-L1 immune checkpoint antibody and chemotherapy.

In yet further embodiments, the subject has received or is selected for having received prior treatment comprising an anti-PD-1 or anti-PD-L1 immune checkpoint antibody and a chemotherapeutic doublet treatment.

In some embodiments, the subject in need has or is selected for having a carcinoma that progressed after a first line immune checkpoint therapy.

In other embodiments, the subject has or is selected for having a carcinoma that progressed after treatment comprising an anti-PD-1 or anti-PD-L1 immune checkpoint antibody.

In other embodiments, the subject has or is selected for having a carcinoma that progressed after chemotherapy.

In additional embodiments, the subject has or is selected for having a carcinoma that progressed after anti-PD-1 or anti-PD-L1 immune checkpoint antibody and chemotherapy doublet treatment.

In some embodiments, the subject in need has or is selected for having a carcinoma that has failed prior treatment with an immune checkpoint inhibitor and platinum-containing doublet treatment.

In some embodiments, the subject in need has or is selected for having a carcinoma that has failed prior treatment with an immune checkpoint inhibitor and a platinum-containing doublet treatment administered simultaneously or sequentially.

In some embodiments, the subject in need has or is selected for having a carcinoma that has failed prior treatment with an anti-PD1 or PDL-1 immune checkpoint antibody and a platinum-containing doublet treatment.

In some embodiments, the subject has or is selected for having a carcinoma that has failed prior treatment comprising an anti-PD-1 immune checkpoint antibody.

In some embodiments, the subject has or is selected for having a carcinoma that has failed prior treatment comprising an PD-L1 immune checkpoint antibody.

In some embodiments, the subject has or is selected for having a carcinoma that has failed prior treatment with an anti-PD-1 immune checkpoint antibody and a platinum-containing doublet treatment.

In some embodiments, the subject has or is selected for having a carcinoma that has failed prior treatment with an anti-PD-L1 immune checkpoint antibody and a platinum-containing doublet treatment.

In some embodiments, the subject has or is selected for having metastatic a carcinoma that has failed prior treatment with an anti-PD-1 or PD-L1 immune checkpoint antibody and a platinum-containing doublet treatment.

In some embodiments, the subject has or is selected for having a carcinoma that has failed prior treatment comprising an anti-PD-1 or anti-PD-L1 immune checkpoint antibody selected from ipilimumab, nivolumab, pembrolizumab, cemiplimab, atezolizumab, avelumab, or durvalumab alone or in combination with platinum-based chemotherapy.

In some embodiments, the subject in need has or is selected for having a carcinoma that has failed prior treatment with ipilimumab, nivolumab, pembrolizumab, cemiplimab, atezolizumab, avelumab, or durvalumab and a platinum-containing doublet treatment.

In some embodiments, the subject in need has or is selected for having a carcinoma that has failed prior treatment with an anti-PD1 or PDL-1 immune checkpoint antibody and a platinum-containing doublet treatment administered simultaneously or sequentially.

In some embodiments, the subject has or is selected for having a carcinoma that has failed prior treatment with pembrolizumab alone or in combination with platinum-based chemotherapy.

In some embodiments, the subject has or is selected for having low expression of PD-L1.

In some embodiments, the subject has or is selected for having one or more lesions with a PD-L1 tumor proportion score (TPS) of ≥50%. L1.

In some embodiments, the subject has or is selected for having one or more lesions with a PD-L1 tumor proportion score (TPS) of <50%.

In some embodiments, the subject has or is selected for having one or more lesions with a PD-L1 tumor proportion score (TPS) of between 1 to 49%.

In some embodiments, the subject has or is selected for having one or more lesions with a PD-L1 tumor proportion score of ≤15%.

In some embodiments, the subject has or is selected for having one or more lesions with a PD-L1 tumor proportion score of ≤1%.

In some embodiments, the subject has or is selected for having one or more lesions with a PD-L1 tumor proportion score of <1%.

In some embodiments, the subject has or is selected for having one or more lesions with a PD-L1 tumor proportion score of 0%.

In some embodiments, the subject has or is selected for having no evidence of PD-L1 expression.

In accordance with the present disclosure, a lesion may be characterized as having low expression of PD-L1 when the PD-L1 tumor proportion score (TPS) of such lesion is, for example, ≤15%.

More particularly, a lesion may be characterized as having low expression of PD-L1 when the PD-L1 TPS of such lesion is, for example, <5%.

More specifically, a lesion may be characterized as having low expression of PD-L1 when the PD-L1 TPS of such lesion is, for example, ≤1%.

Even more specifically, a lesion may be characterized as having low expression of PD-L1 when the PD-L1 TPS of such lesion is, for example, <1%.

In accordance with the present disclosure, a lesion may be characterized as having low expression of PD-L1 when the PD-L1 combined positive score (CPS) of such lesion is, for example, <10%.

More particularly, a lesion may be characterized as having low expression of PD-L1 when the PD-L1 CPS of such lesion is, for example, <5%.

More specifically, a lesion may be characterized as having low expression of PD-L1 when the PD-L1 CPS of such lesion is, for example, ≤1%.

Even more specifically, a lesion may be characterized as having low expression of PD-L1 when the PD-L1 CPS of such lesion is, for example, <1%.

In accordance with the present disclosure, a lesion is characterized as having no evidence of PD-L1 expression when the PD-L1 TPS or CPS of such lesion of is 0% or when PD-L1 expression undetectable.

In some embodiments, the tumor proportion score is determined at baseline.

In some embodiments, the tumor proportion score is determined prior to administration of the anti-cancer therapy of the present disclosure.

In some embodiments, the tumor proportion score is determined after first-line therapy.

In some embodiments, the tumor proportion score is determined at the onset of the anti-cancer therapy.

In some embodiments, the PD-L1 the tumor proportion score is determined by a scientist or pathologist.

In some embodiments, the PD-L1 the tumor proportion score is determined in accordance with drug regulator's guidance (e.g., FDA, EMA and the like).

In some embodiments, the subject has or is selected for having a carcinoma with one or more lesions having low expression of PD-L1.

In some embodiments, the subject has or is selected for having a carcinoma with no evidence of PD-L1 expression.

In some embodiments, the subject has or is selected for having a carcinoma that is not eligible for or would unlikely benefit from treatment with an anti-PD-1 or anti-PD-L1 immune checkpoint inhibitor.

In some embodiments, the subject has or is selected for having a carcinoma that is not eligible for or would unlikely benefit from treatment with an anti-PD-1 or anti-PD-L1 immune checkpoint antibody.

A subject may be characterized as being not eligible for treatment with a PD-1 or PD-L1 immune checkpoint inhibitor based on objective criteria such as for example, prior failure of treatment comprising a PD-1 or PD-L1 immune checkpoint inhibitor, level of expression of PD-1 or PD-L1, level of immune cell infiltration in the tumor environment and the like.

A subject may be characterized as being unlikely to benefit from treatment with a PD-1 or PD-L1 immune checkpoint inhibitor based on subjective criteria such as, physician assessment of risks and benefits, overall clinical condition of the subject, and the like.

In accordance with the present disclosure, the subject has or is selected for having a metastatic carcinoma with one or more lesions with low expression of PD-L1, no evidence of PD-L1 expression or that is not eligible to treatment comprising an anti-PD-1 or anti-PD-L1 immune checkpoint antibody.

In some embodiments, the subject in need is or is selected for not being immunosuppressed.

In some embodiments, the subject is or selected for being immunocompetent.

In accordance with the present disclosure, the subject is not or is selected for not being immunosuppressed or has not received an immunosuppressive medication within 1 to 14 days prior to treatment.

In some embodiments, the subject in need has not received an immunosuppressive medication within 14 days, 7 days, 6 days, 5 days, 4 days, 3 days, 2 days or 1 day prior to treatment. In some embodiments, the subject in need may have received corticosteroids prior to treatment.

In some embodiments, the subject has or is selected for not having received an immunosuppressive medication within at least 7 days prior to treatment.

In some embodiments, the subject in need has not received prior treatment with docetaxel.

In some embodiments, the subject in need is treated for at least two cycles of treatment.

### PD-L1 Expression and Immune Cells Infiltration

PD-L1 expression may be determined using methods known to a person skilled in the art.

In an exemplary embodiment, the PD-L1 tumor proportion score (TPS) is assessed with the 22C3 antibody (Agilent Technologies, Carpinteria, CA, code SK006).

In another exemplary embodiment, the PD-L1 TPS is assessed with the 28-8 antibody (Agilent Technologies, Carpinteria, CA, code SK005: Abcam, Toronto, Canada, Cat. No. Ab205921).

In yet another exemplary embodiment, PD-L1 TPS is assessed with the SP263 antibody Roche Diagnostic Ref. 740-4907.

In a further exemplary embodiment, the PD-L1 TPS is assessed with the SP142 antibody (Abcam, Toronto, Canada, Cat. No. Ab228462).

In a further exemplary embodiment, the PD-L1 TPS is assessed with the 73-10 antibody (clone MKP1A07310; Merck KgaA, Darmstadt, Germany: Abcam, Toronto, Canada, Cat. No. Ab228415).

The PD-L1 expression may be assessed using an anti-PD-L1 antibody suitable for the type of carcinoma that the subject has.

The PD-L1 expression is usually assessed in tumor cells. However, the PD-L1 expression may be assessed both in tumor cells and in tumor-infiltrating immune cells.

In some instances, the PD-L1 expression is assessed by immunohistochemistry.

In other instances, the PD-L1 expression is assessed by fluorescence assays, such as for example, by quantitative fluorescence or fluorescence in-situ hybridization.

In some embodiments, the assay is an assay approved by at least one regulatory body.

In accordance with the present disclosure, the assay is a United States Food and Drug Administration (FDA)-approved diagnostic test (Dx).

In accordance with the present disclosure, the assay is an FDA-approved test for research (Rx).

In an exemplary embodiment, the assay is based on PD-L1 IHC 22C3 pharmDx (Agilent Technologies, Carpinteria, CA, code SK006).

In another exemplary embodiment, the assay is based on PD-L1 IHC 28-8 pharmDx ((Agilent Technologies, Carpinteria, CA, code SK005)).

In yet another exemplary embodiment, the assay is based on Ventana PD-L1 (SP263) (Roche Diagnostic Ref. 740-4907).

In some aspects and embodiments, the immunohistochemistry assay is performed in accordance with manufacturer's instructions.

In some embodiments, the assay uses Autostainer Link 48 immunohistochemistry platform (Dako).

In some embodiments, the assay uses the Omnis immunohistochemistry platform (Dako).

In some embodiments, the assay is performed with the Bond-III immunohistochemistry platform (Leica).

In some embodiments, the assay uses the BenchMark ULTRA immunohistochemistry platform (Ventana).

The assay results are interpreted using the manufacturer's interpretation manual.

Exemplary assays are described in Munari, E., et al., 2021 Marchetti, A. et al., 2017, Yoshikawa, K. et al., 2021, the entire content of all of which is incorporated herein by reference.

In some embodiments, the absence or presence of immune cells in the tumor microenvironment may be confirmed by tumor biopsy.

In other embodiments, the absence or presence of immune cells in the tumor microenvironment may be confirmed by in vivo imaging (e.g., magnetic resonance imaging, e.g., see Jiang X. et al., 2020).

A tumor may be characterized as “immunologically cold” when the tumor microenvironment is not sufficiently infiltrated by immune cells (especially by lymphocytes) or when the tumor microenvironment is not inflamed. In contrast, a tumor may be characterized as “immunologically warm” or “immunologically hot” when infiltration of immune cells (especially by lymphocytes) in the tumor microenvironment is observed or when the tumor shows sign of inflammation. Failure to respond to immunotherapy may be a sign that the tumor or lesion is “immunologically cold”. As such, the present disclosure relates to a method of treating a subject having one or more lesions that do not respond to immunotherapy.

Generally, a pathologist, a technologist, a trained scientist or trained technician equipped with proper reagents and/or apparatus may be able to determine the absence or presence of immune cells in the tumor microenvironment and may thus be able to evaluate whether a tumor is “immunologically cold”, “immunologically warm” or “immunologically hot”.

### Kits

The present disclosure provides, in some aspects and embodiments, a kit comprising an anti-cancer therapy for use in a method described herein and for treating subjects described herein.

In some embodiments, the kit comprises one or more containers comprising at least one dose of an anti-clusterin antibody or antigen binding fragment thereof, one or more containers comprising at least one dose of a chemotherapeutic for use in combination therapy and a package insert comprising instructions for treating a subject in need.

In some embodiments, the kit comprises one or more containers comprising at least one dose of the medicament disclosed herein and a package insert comprising instructions for treating a subject in need.

In embodiments, the kit comprises one or more containers comprising at least one dose of an anti-clusterin antibody or antigen binding fragment thereof, one or more containers comprising at least one dose of docetaxel for use in combination therapy and a package insert comprising instructions for treating a subject in need.

In accordance with the present disclosure, the anti-clusterin antibody or antigen binding fragment thereof and docetaxel are provided in separate containers.

In accordance with the present disclosure, the antibody or antigen binding fragment thereof is as described herein.

In some embodiments, the package insert states that the combination therapy is intended for treatment of a subject having a carcinoma.

In some embodiments, the package insert states that the combination therapy is intended for treatment of a subject having metastatic carcinoma.

In some embodiments, the package insert states that the combination therapy is intended for treatment of a subject having a carcinoma that progressed after a first line immune checkpoint therapy.

In some embodiments, the package insert states that the combination therapy is intended for treatment of a subject having a carcinoma that has failed prior treatment with an immune checkpoint therapy and a platinum-containing doublet treatment.

In some embodiments, the package insert states that the combination therapy is intended for treatment of a subject having a carcinoma that has failed prior treatment with an immune checkpoint therapy and a platinum-containing doublet treatment administered either simultaneously or sequentially.

In some embodiments, the package insert states that the combination therapy is intended for treatment of a subject having a carcinoma that has failed prior treatment with an anti-PD1 or PDL-1 immune checkpoint antibody and a platinum-containing doublet treatment.

In some embodiments, the package insert states that the combination therapy is intended for treatment of a subject having a carcinoma that has failed prior treatment with an anti-PD1 or PDL-1 immune checkpoint antibody and a platinum-containing doublet treatment administered either simultaneously or sequentially.

In some embodiments, the package insert states that the combination therapy is intended for treatment of a subject having a carcinoma that has failed prior treatment with ipilimumab, nivolumab, pembrolizumab, cemiplimab, atezolizumab, avelumab, or durvalumab and a platinum-containing doublet treatment administered either simultaneously or sequentially.

In some embodiments, the package insert states that the combination therapy is intended for treatment of a subject having non-small cell lung cancer (NSCLC).

In some embodiments, the package insert states that the combination therapy is intended for treatment of a subject having advanced NSCLC.

In some embodiments, the package insert states that the combination therapy is intended for treatment of a subject having stage III NSCLC.

In some embodiments, the package insert states that the combination therapy is intended for treatment of a subject having stage IV NSCLC.

In some embodiments, the package insert states that the combination therapy is intended for treatment of a subject having breast cancer.

In some embodiments, the package insert states that the combination therapy is intended for treatment of a subject having metastatic breast cancer.

In some embodiments, the package insert states that the combination therapy is intended for treatment of a subject having prostate cancer.

In some embodiments, the package insert states that the combination therapy is intended for treatment of a subject having metastatic prostate cancer.

In some embodiments, the package insert states that the combination therapy is intended for treatment of a subject having gastric cancer.

In some embodiments, the package insert states that the combination therapy is intended for treatment of a subject having metastatic gastric cancer.

In some embodiments, the package insert states that the combination therapy is intended for treatment of a subject having head and neck cancer.

In some embodiments, the package insert states that the combination therapy is intended for treatment of a subject having metastatic head and neck cancer.

In some embodiments, the package insert states that the combination therapy is intended for treatment of a subject having thyroid cancer.

In some embodiments, the package insert states that the combination therapy is intended for treatment of a subject having metastatic thyroid cancer.

In some embodiments, the package insert states that the combination therapy is intended for treatment of a subject having ovarian cancer.

In some embodiments, the package insert states that the combination therapy is intended for treatment of a subject having metastatic ovarian cancer.

In some embodiments, the package insert states that the combination therapy is intended for treatment of a subject that is not immunosuppressed has not received an immunosuppressive medication within 14 days, 7 days, 6 days, 5 days, 4 days, 3 days, 2 days or 1 day prior to treatment.

In some embodiments, the package insert states that the combination therapy is intended for treatment of a subject that has not received prior treatment with docetaxel.

In some embodiments, the package insert states that the combination therapy is for administration essentially over the entire course of the treatment period or throughout the treatment period.

### EXAMPLES

### Example 1—Non-Clinical Pharmacokinetics

The pharmacokinetics of AB-16B5 (humanized 16B5) was investigated in single and repeat-dose studies in Sprague-Dawley rats and Rhesus monkeys. In general, peak serum AB-16B5 concentrations were reached shortly after the start of infusion. In rats, AB-16B5 systemic exposure over 24 hours increased with increasing dose levels in a dose proportional manner. There was an approximate 70% increase in systemic exposure following repeated dosing from Day 1 to Day 26 which is consistent with the elimination half-life of AB-16B5 and the dosing intervals. In monkeys, AB-16B5 systemic exposure increased with increasing dose levels in a generally greater than dose proportional manner. For repeated dosing, there were no notable differences in systemic exposure to AB-16B5 whether it was administered once weekly or twice weekly. There was no evidence of drug accumulation in terms of Cₘₐₓ while a lower extent of accumulation was observed for AUC₀₋₇₂. Interestingly, after single dose administration of AB-16B5 in Rhesus monkeys, sCLU serum concentrations decreased significantly following dosing. The sCLU concentrations were generally back to their baseline levels after 3 to 5 days. The concurrent decrease in sCLU concentrations immediately following dosing with AB-16B5 is consistent with an antigen-sink phenomenon.

### Example 2—Non-Clinical Toxicity

In Sprague-Dawley rats, weekly administration of AB-16B5 (humanized 16B5) did not result in any treatment-related clinical signs or any treatment-related changes in body weight, food consumption, ophthalmology, clinical pathology, organ weights. Similarly, there were no treatment-related macroscopic changes. A very low incidence of minimal lymphoid hypercellularity was noted in the mesenteric lymph node (2/20) and mandibular lymph node (1/20) of animals from the high dose group. This microscopic finding was not seen in any recovery animals following the 28-day recovery period indicating complete reversibility. This finding most likely reflects a slight antigenic stimulation by AB-16B5 in these 2 regional lymph nodes. The No Observable Adverse Effect Level (NOAEL) was therefore determined to be 100 mg/kg/dose.

In Rhesus monkeys, weekly administration of AB-16B5 did not result in any signs of overt toxicity. Few episodes of emesis were observed during the infusion of AB-16B5 in animals of all dose levels. There were no treatment-related effects observed on body weight, ophthalmology, electrocardiogramand organ weight. Slight decreases in mean white blood cell counts were seen at 60 mg/kg (males only) and 100 mg/kg (males and females). Slight changes in mean albumin, globulin, A/G and phosphorus levels were also seen at 20, 60 and/or 100 mg/kg. Although potentially related to AB-16B5, these changes were minor, reversible, lacked a dose-response relationship and were not associated with any histopathology correlates. Consequently, they were not considered to be toxicologically significant. Hepatocellular vacuolation was noted in the liver of 2 animals dosed at 100 mg/kg (1 main and 1 recovery animal). The relationship of this finding to treatment with AB-16B5 remains uncertain because of the low incidence and because this finding is known to occur spontaneously. In absence of clinical pathology (liver enzymes) and organ weight (liver weight) correlates, this finding was considered to be an adaptive change and non-adverse. The NOAEL was therefore determined to be 100 mg/kg/dose.

In addition, a tissue cross-reactivity study was conducted to determine the potential cross-reactivity of AB-16B5 with frozen tissues from human, Rhesus monkey, and Sprague-Dawley rat tissues. AB-16B5 staining at 2 and 10 μg/mL was observed in a number of tissue elements in human, Rhesus monkey, and Sprague-Dawley rat tissue panels. However, no membrane staining was observed in any of the tissues examined in this study supporting the lack of potential toxicological concern due to off-target binding.

### Example 3—Phase I Clinical Study

A first-in-human phase I trial to assess safety and tolerability of AB16B5 (humanized 16B5) was carried out (clinical trial registry number: NCT2412462) (Ferrario et al., 2017) in subjects with a histologically or cytologically confirmed advanced solid malignancy that has been refractory to prior therapy and is unlikely to benefit from known therapies. AB-16B5 has been administered in 15 subjects enrolled in a phase 1, single-center, open-label, dose-escalation study (AB-16B5-101).

The dose levels of AB-16B5 that were assessed during the study were 1.5, 3.0, 6.0, 9.0, and 12.0 mg/kg. The dose escalation was performed using 2 dose escalation schemes: the accelerated dose escalation scheme and the standard dose escalation scheme. Only 1 subject was enrolled in each of the first 2 cohorts as per protocol (accelerated dose escalation scheme). The last cohort (12 mg/kg) was expanded to at least 6 subjects as per protocol.

Subjects received AB-16B5 by a 60-minute IV infusion once weekly on Days 1, 8 and 15 of each cycle of treatment. One cycle of treatment consisted of 21 days.

### Subject Characteristics

Majority of AB-16B5-treated subjects were female (10 of the 15 subjects, 67%) and the mean subject age was 61 years old (range: 32-79 years old). The most common cancer diagnoses found in the subject population were breast, colorectal, prostate and thyroid (2 subjects each, 13%). Other tumor types were found in no more than 1 subject each (7%) and included endometrium, gastric, lung, ovarian, pancreatic, soft-tissue sarcoma and vulvar melanoma.

### Time on Study

Fourteen subjects (93%) received at least 2 cycles of treatment (range: 2 to 17 cycles). One subject (Subject 004) completed 17 cycles of treatment. This subject received AB-16B5 at 6 mg/kg from Cycle 1 to cycle 7 and the dose was escalated to 9 mg/kg thereafter. Thirteen subjects (87%) discontinued the study due to disease progression: 2 subjects discontinued due an adverse event (Subject 004 with Grade 3 bronchial obstruction; associated with disease progression and Subject 011 with Grade 3 biliary obstruction). Subject 011 in cohort 12 mg/kg was discontinued after one dose of AB-16B5 by the Investigator and was replaced by a new subject: however, Subject 011 was not included in the RECIST evaluable population. The mean number of cycles received in the RECIST-evaluable population was 4.4 cycles.

### Safety Profile

The administration of AB-16B5 given as a 60-minute IV weekly infusion was safe and well tolerated at doses up to 12 mg/kg. No dose-limiting toxicity was observed.

A total of 167 Aes were reported. Among those Aes, 68 out of 167 Aes were related (possibly, probably or definitely) to AB-16B5 and twelve (12) were of Grade 3 to 5.

The most frequently reported Aes (all causalities) were nausea, abdominal pain, back pain, vomiting, chills, dyspnea, constipation and pruritus.

There was no dose-related trend in the incidence of these Aes. The most frequently reported Aes related to AB-16B5 were nausea, pruritus, headache and rash. There was no dose-related trend in the incidence of drug-related Aes. The drug-related Aes were mainly of Grade 1 or 2.

There were 12 Aes of Grade 3 to 5 that were reported by 8 subjects. Only 2 of them were related to AB-16B5 (Grade 3 infusion related reaction and Grade 3 rash). Two deaths were reported in the study (Subjects 004 and 014). In both cases, death was associated with the progression of the disease.

A total of 5 of 15 (33%) subjects reported 6 SAEs. All SAEs were considered unrelated to AB-16B5. Two subjects (13%) discontinued the study due to an adverse event: Subject 004 (Grade 3 bronchial obstruction; associated with disease progression) and Subject 011 (Grade 3 biliary obstruction).

### Antitumor Activity and Best Overall Response

The best overall response (BOR) in the RECIST-evaluable population is presented in Table 1 below:

TABLE 1
Tumor Response at Last Assessment and Best Overall Response

| | | | | Tumor Response at | | |
|---|---|---|---|---|---|---|
| | | | | Last Assessment | | Best |
| Dose | | Primary | Total | Target | Non-Target | Overall |
| (mg/kg) | Subject | Tumor | Cycle | Lesions | Lesions | Response |
| 1.5 | 001 | Pancreatic | 2 | PD | PD (New | PD |
| | | | | | lesion) | |
| 3.0 | 002 | Endometrium | 4 | PD | Non- | SD |
| | | | | | CR/Non-PD | |
| 6.0 | 003 | Soft Tissue | 2 | SD | PD (New | PD |
| | | Sarcoma | | | lesion) | |
| 6.0 | 004 | Thyroid | 17 | SD | Non- | SD |
| | | | | | CR/Non-PD | |
| 6.0 | 005 | Prostate | 2 | PD | Non- | PD |
| | | | | | CR/Non-PD | |
| 9.0 | 006 | Breast | 3 | NA | Non- | SD |
| | | | | | CR/Non-PD | |
| 9.0 | 007 | Gastric | 8 | SD | PD (New | SD |
| | | | | | lesion) | |
| 9.0 | 008 | Thyroid | 4 | SD | PD (New | SD |
| | | | | | lesion) | |
| 12.0 | 009 | Lung | 6 | SD | PD (New | SD |
| | | | | | lesion) | |
| 12.0 | 010 | Colorectal | 4 | PD | PD (New | SD |
| | | | | | lesion) | |
| 12.0 | 012 | Breast | 2 | SD | PD | PD |
| 12.0 | 013 | Prostate | 2 | SD | PD (New | PD |
| | | | | | lesion) | |
| 12.0 | 014 | Vulvar | 2 | SD | PD (New | PD |
| | | Melanoma | | | lesion) | |
| 12.0 | 015 | Ovarian | 4 | SD | PD (New | SD |
| | | Cancer | | | lesion) | |

The best overall response in this population was stable disease (SD) for 8 subjects (57%) and progressive disease (PD) for 6 subjects (43%). AB-16B5 treatment lasting 4 or more cycles might suggest a clinical benefit.

It was noted that the majority of subjects who had progressed during treatment progressed due to the appearance of a new lesion. Indeed, the tumor response at the last assessment for Subjects 003, 007, 008, 009, 013, 014 and 015 was consistent with stable disease at the target lesions but progressive disease at the non-target lesions because of the appearance of a new lesion. This finding is consistent with the mechanism of action of AB-16B5. Indeed, new lesions contain tumor cells mostly of epithelial phenotype as a consequence of mesenchymal to epithelial transition. Hence, these new lesions contain low amounts of TA-sCLU since they have not been exposed to EMT-promoting conditions such as chemotherapy or hypoxia which limits exposure to AB-16B5.

### AB-16B5-101: Pharmacodynamics

Tumor biopsies were collected during the standard dose escalation to investigate the presence of AB-16B5 within the tumor. A total of 4 pre-treatment and 5 post-treatment tumor biopsies were suitable for analysis. These post-treatment tumor biopsies were collected following dosing on Cycle 2 Day 15 (between Cycle 2 Day 15 and Cycle 2 Day 18). The biopsy sites were liver (n=2), lymph node (n=2), bladder, skin and vulva.

TABLE 2
Detection of AB-16B5 in Tumor Biopsies

| | | Site of | Pre-Treatment | Post-Treatment |
|---|---|---|---|---|
| Subject | Diagnosis | Biopsy | Biopsy | Biopsy |
| 009 | Lung | Liver | NA | Positive |
| 010 | Colorectal | Liver | NA | Positive |
| 011 | Colorectal | Lymph node | Negative | NA |
| 012 | Breast | Skin | Negative | Positive |
| 013 | Prostate | Bladder | NA | Positive |
| 014 | Vulvar | Vulva | Negative | NA |
| | melanoma | | | |
| 015 | Ovarian | Lymph node | Negative | Positive |

NA: Not available

All pre-treatment tumor biopsies were negative for the presence of AB-16B5 while the presence of AB-16B5 was confirmed in all post-treatment tumor biopsies analyzed. This confirms that AB-16B5 preferentially binds to TA-sCLU and that the tumor potentially may represent an important reservoir.

Immunohistochemistry studies were conducted on paired tumor biopsies to determine if the treatment with AB-16B5 was associated with a reacquisition of an epithelial phenotype. Among the 4 evaluable paired tumor biopsies, two cases showed an increase in E-cadherin expression (Subjects 006 and 007) one of which was combined with a loss of vimentin expression (Subject 007). One case (Subject 013) showed strong epithelial characteristics in the pre-treatment biopsy that did not change with AB-16B5 treatment.

TABLE 3
Expression of E-cadherin and Immune Response of AB-16B5

| | | | E-cadherin | Necrosis | |
|---|---|---|---|---|---|
| | | Site of | Expression in | Pre- | Post- |
| Subject | Diagnosis | Biopsy | Post-Treatment | Treatment | Treatment |
| 006 | Breast | Skin | Increase | 0% | 10% |
| 007 | Gastric | Gastric | Increase | 0% | 0% |
| 012 | Breast | Skin | Decrease | 0% | 5% |
| 013 | Prostate | Bladder | No change | 0% | 10% |

| | | | | |
|---|---|---|---|---|
| 015 | Ovarian | Lymph | Not evaluable | Not evaluable |
| | | node | | |

Based on these limited results, AB-16B5 treatment may increase epithelial characteristics and induce necrosis.

### AB-16B5-101: Pharmacokinetics

Plasma PK parameters for AB-16B5 were derived from the concentration-time profiles following the first dose of Cycle 1 and the first dose of Cycle 2 using a non-compartmental analysis (NCA) with Phoenix WinNonlin.

On Cycle 1, median AB-16B5 Tₘₐₓ were similar across the dose levels. Mean Cₘₐₓ and AUC₀₋ₜ increased with the dose. This increase seemed to be dose proportional over the dose range of 3.0 to 12.0 mg/kg. AB-16B5 terminal phase was well characterized with overall residual values <20.00%. The characterization of the terminal phase was improved at 12.0 mg/kg when compared to that observed at the other doses. Mean T_{half} was similar at the 1.5, 3.0 and 6.0 mg/kg dose levels, but were slightly higher at the 9.0 and 12.0 mg/kg dose levels.

On Cycle 2, median AB-16B5 Tₘₐₓ were similar across the dose levels, with values ranging from 1.00 to 1.08 h. Mean Cₘₐₓ and AUC₀₋ₜ increased with the doses. This increase seemed to be dose proportional over the dose range of 3.0 to 12.0 mg/kg. AB-16B5 terminal phase on Cycle 2 was well characterized with overall residual values <15.00%. The characterization of the terminal phase was improved at 9.0 mg/kg and 12.0 mg/kg (<1.00%) when compared to that observed at lower doses. Mean t_{1/2} was similar across the dose range of 1.5 mg/kg to 6.0 mg/kg while mean T_{half} were slightly higher at the 9.0 and 12.0 mg/kg dose levels.

No significant accumulation of AB-16B5 was observed between Cycle 1 and Cycle 2.

A summary of the pharmacokinetic parameters is presented in Table 4.

TABLE 4
Summary of Mean (CV %) Pharmacokinetic Parameters of AB-16B5

| | | 1.5 mg/kg | 3.0 mg/kg | 6.0 mg/kg | 9.0 mg/kg | 12.0 mg/kg |
|---|---|---|---|---|---|---|
| Parameters | | (N = 1) | (N = 1) | (N = 3) | (N = 3) | (N = 7) |
| AUC₀₋ₜ | Cycle 1 | 230,321 | 645,602 | 1,357,773 | 2,083,091 | 4,045,786 |
| (ng*h/mL) | | (NC) | (NC) | (18.6) | (39.6) | (13.5) |
| | Cycle 2 | 188,584 | 688,934 | 1,351,156 | 2,459,988 | 4,147,243 |
| | | (NC) | (NC) | (11.7) | (17.0) | (18.6) |
| Cₘₐₓ | Cycle 1 | 17,958 | 50,736 | 117,663 | 162,103 | 256,977 |
| (ng/mL) | | (NC) | (NC) | (14.4) | (21.4) | (14.7) |
| | Cycle 2 | 15,662 | 61,466 | 120,466 | 163,840 | 230,268 |
| | | (NC) | (NC) | (9.9) | (10.4) | (23.5) |
| Tₘₐₓ | Cycle 1 | 1.17 | 1.08 | 1.00 | 2.00 | 1.12 |
| (h)^{a} | | (1.17-1.17) | (1.08-1.08) | (1.00-1.00) | (2.00-2.00) | (1.00-2.05) |
| | Cycle 2 | 1.00 | 1.05 | 1.03 | 1.08 | 1.04 |
| | | (1.00-1.00) | (1.05-1.05) | (1.00-1.42) | (1.00-2.00) | (1.00-2.00) |
| T_{half} | Cycle 1 | 9.35 | 7.39 | 6.99 | 10.78 | 11.99 |
| (h) | | (NC) | (NC) | (5.3) | (68.3) | (43.3) |
| | Cycle 2 | 7.97 | 6.82 | 7.70 | 13.75 | 21.04 |
| | | (NC) | (NC) | (8.2) | (53.6) | (17.4) |

^{a}Values are mean (CV %) except for Tₘₐₓ where the median (range) is presented;
NC: Not calculated

### Tumor Biomarkers

Several well-established circulating biomarkers were evaluated to monitor the response to therapy. They included CA 15-3, CA-125, CA 19-9, CEA, LDH and PSA.

In general, tumor biomarker levels increased over time which correlated with disease progression. Interestingly, the level of CEA remained stable or decreased in few patients.

### CONCLUSION

Patients received between 1 and 53 weekly doses (median: 9 doses). The most frequently reported treatment-emergent adverse events (TEAEs, all causalities) were nausea, abdominal pain, back pain, vomiting, chills, constipation and pruritus. Most of the Aes were of Grade 1 or 2. Among the Aes≥Grade 3, only 2 (Grade 3 infusion-related reaction and rash) were judged related to AB-16B5. No dose-limiting toxicities were identified during the first cycle of treatment for any patient 5 serious Aes were reported (sepsis, pyrexia, dyspnea, intra-abdominal hemorrhage and main stem bronchus obstruction), none of which were judged related to the study treatment. PK analysis across all dose levels confirmed that systemic exposure to AB-16B5 increased in a dose proportional manner. The presence of AB-16B5 at the tumor site was confirmed in all 5 pts where a post-treatment tumor lysate could be generated. Biomarker analysis in paired tumor biopsies provided some evidence for EMT inhibition as seen by increased E-cadherin expression after treatment with AB-16B5 in 2 pts. In one of these 2 pts with advanced gastric cancer, this was also accompanied by a loss of vimentin expression. This patient had stable disease (SD) with clinical benefit and remained on treatment for 24 weeks. Another patient with follicular thyroid cancer had SD for almost 1 year.

Weekly infusions of AB-16B5 are well tolerated up to 12 mg/kg. Early correlative studies on tumor tissue provide evidence of molecular modulation of the tumor environment in humans.

### Example 4—Effect of AB-16B5 on Infiltration of Immune Cells in the Tumor Microenvironment

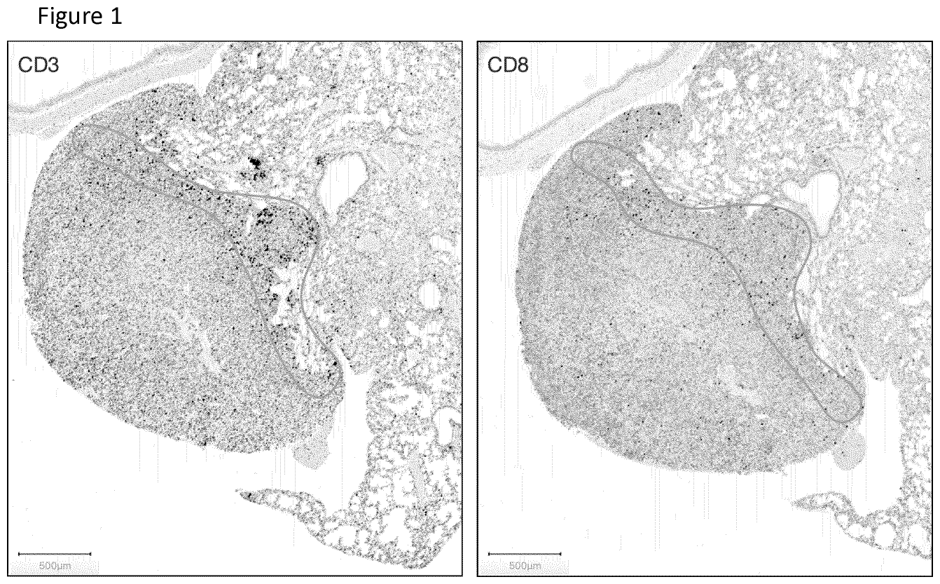

Balb/c mice were orthotopically implanted with 5×10⁵ 4T1 cells in the 4^{th} mammary fat pad. Animals received IP saline treatment thrice weekly. The primary tumor was surgically removed at Day 16 post-implantation. The animals were sacrificed at Day 36 and the lungs were excised. Tissues were fixed in paraformaldehyde and processed for paraffin embedding. Tissue sections were probed with anti-mouse CD3, anti-mouse CD8 and anti-mouse B220 antibodies. Signals were revealed with specific secondary antibodies conjugated with horseradish peroxidase and counter stained with hematoxylin and eosin. Results presented in FIG. 1 indicate that the 4T1 lung metastases create an immune cold microenvironment which prevents the infiltration of B and T lymphocytes in tumors. Delineated regions indicate that CD3 and CD8 T lymphocytes are restricted in the tumor margin as a consequence of EMT.

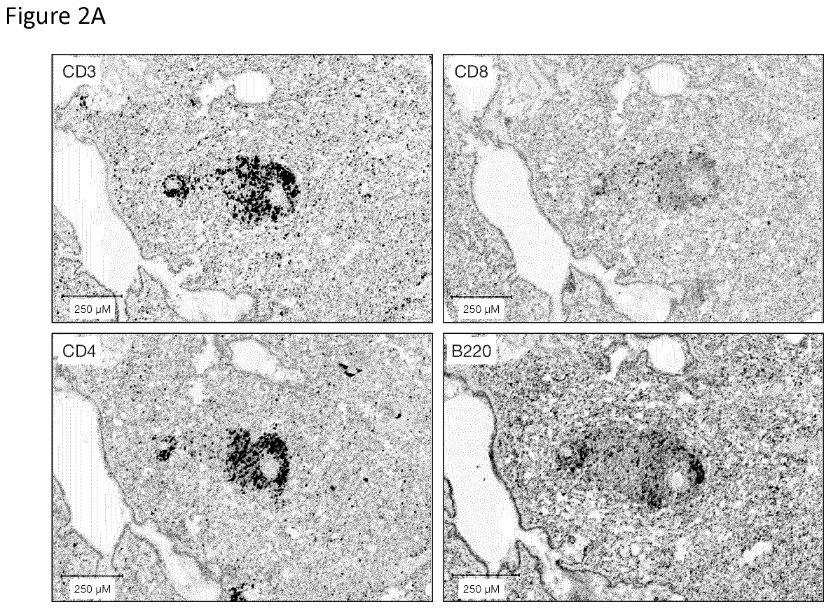

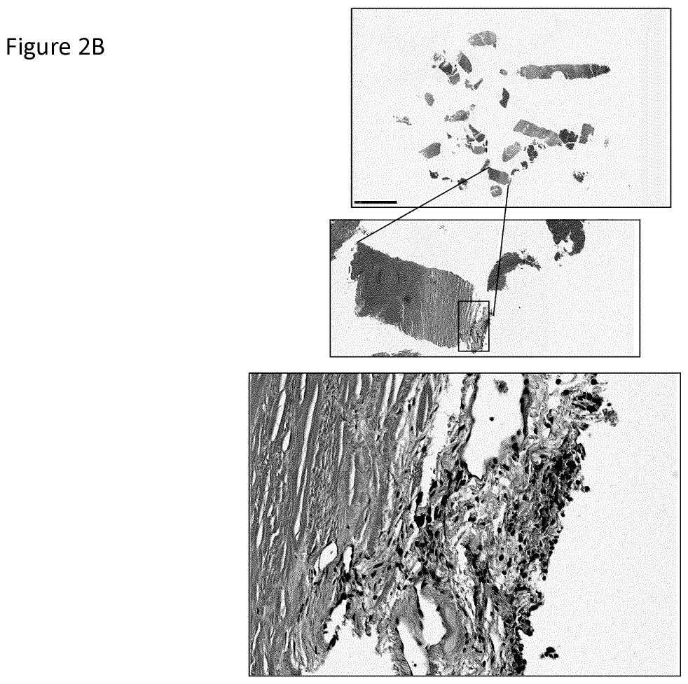

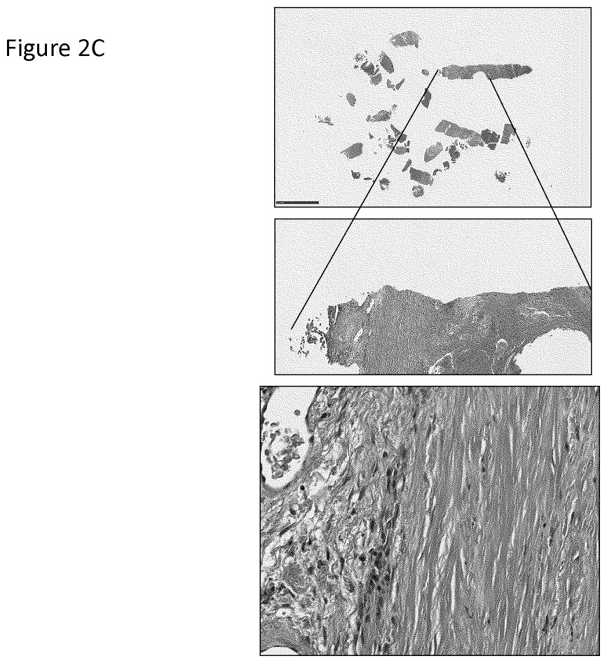

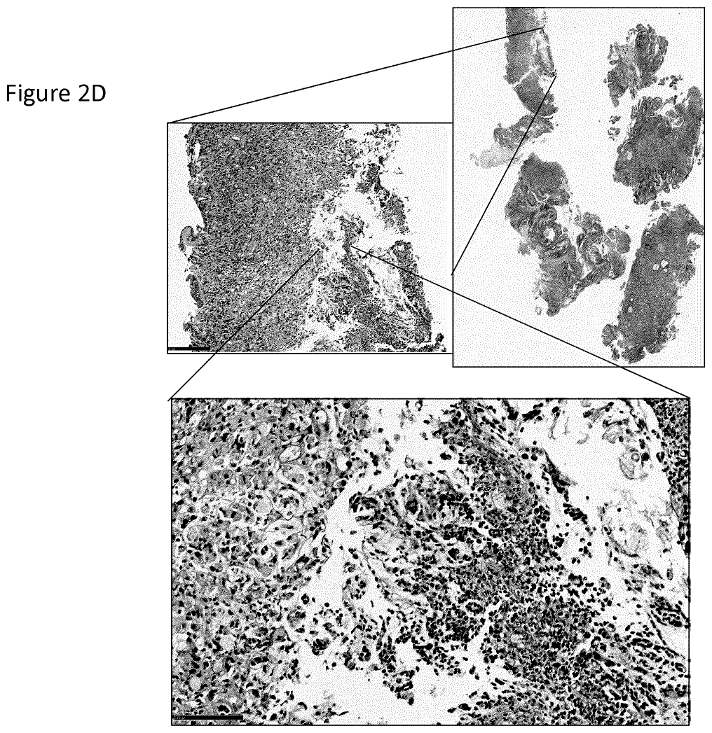

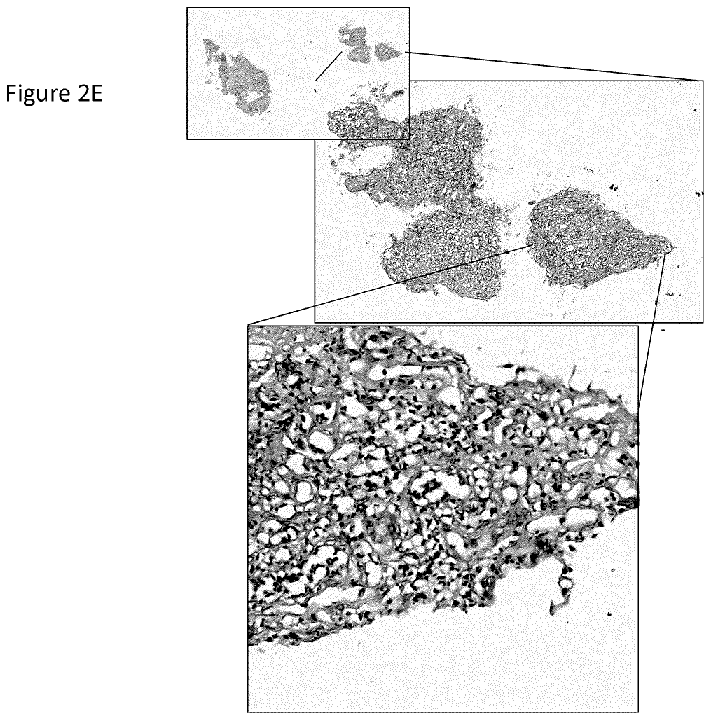

Animals bearing 4T1 tumors were treated with the AB-16B5 antibody (murine 16B5) thrice a week IP at 10 mg/kg. The primary tumor was surgically removed at Day 16 post-implantation. The animals were sacrificed at Day 36 and the lungs were excised. Tissues were fixed in paraformaldehyde and processed for paraffin embedding. Tissue sections were probed with anti-mouse CD3, anti-mouse CD8 and anti-mouse B220 antibodies. Signals were revealed with specific secondary antibodies conjugated with horseradish peroxidase and counter stained with hematoxylin and eosin. Results presented in FIG. 2A indicate that there were fewer and much smaller lung metastases that were densely infiltrated with CD3 and CD8 T cells. There was also evidence of plasmocytes penetration in 16B5-treated tumors.

AB-16B5 thus allows infiltration of immune cells in the tumor microenvironment in immunocompetent mice. AB-16B5 might represent a new therapeutic avenue to create a warmer tumor environment to stimulate a strong immune response against tumors.

In parallel human tumor biopsies of patients treated with AB-16B5 as single agent were analyzed (FIGS. 2B-2E). Needle biopsies obtained from a patient with metastatic thyroid cancer and form a patient with inoperable metastatic gastric cancer were sectioned and stained with hematoxylin and eosin. An on-treatment biopsy from a patient with thyroid cancer metastasic to the lungs was obtained after the second cycle treatment with AB-16B5. As shown in FIG. 2B, essentially all tumor fragments were necrotic. A lymphoplasmacytic infiltrate was observed along the edge of the fragment on display. Hemosiderin-laden macrophages were observed inside necrotic areas some reflecting red blood cell extravasation associated with necrosis (not shown). FIG. 2C shows a perivascular infiltrate composed of plasma cells along the edge of tumor fragment from the same patient. On display one finds. The analysis of the pre-treatment biopsy from the metastatic gastric cancer case showed several fragments of gastric mucosa infiltrated by a diffuse poorly differentiated gastric cancer (signet-ring cells) The fragment on display showed foci of necrosis with a predominantly acute neutrophilic infiltrate. FIG. 2E shows the on-treatment biopsy obtained after the second cycle of treatment with AB-16B5 comprised of three tumor fragments. The larger fragment consisted of normal superficial gastric mucosa and that the small fragments were infiltrated by a mix neutrophilic and mononucleated immune cells infiltrate.

### Example 5—Effect of the Combination Therapy of AB-16B5 and Docetaxel on Infiltration of Immune Cells in the Tumor Microenvironment

An immunocompetent mouse cancer model was selected for testing the extent of the immune response upon treatment with AB-16B5 monotherapy or combination of AB-16B5 and docetaxel using the murine 16B5.

Five groups, each consisting of 10 female Balb/c mice were assigned to this study (see Table 5 below). All animals received subcutaneous implantation of 4T1 mouse mammary carcinoma cells in the 4th inguinal mammary gland. Treatment was initiated on the day of implantation (defined as Day 1). Animals from Group 1 (Gr. 1) received IP treatment of saline vehicle control twice a week for the duration of the study. Animals from Group 2 (Gr. 2) received 10 mg/kg of docetaxel weekly for five weeks by IP administration. Animals from Group 3 (Gr. 3) received 10 mg/kg of docetaxel weekly for two weeks and 10 mg/kg of AB-16B5, twice weekly for five weeks. Animals from Group 4 (Gr. 4) received 10 mg/kg of docetaxel weekly and 5 mg/kg of 16B5 twice weekly each over the course of a five weeks treatment. Animals from Group 5 (Gr. 5) received AB-16B5 twice weekly for five weeks. On Day 36, the primary tumors were excised and on Day 37, the animals were sacrificed and the number of grossly visible metastatic nodules on the surface of the lungs was counted.

TABLE 5
Dosing schedule:

| | | | | | Week 3 to 5 | | |
|---|---|---|---|---|---|---|---|
| | | Week 1 and 2 | | | | Day 2 | |
| Group # | Treatment | Day 1 | Day 2 | Day 4 | Day 1 | or 3* | Day 4 |
| Group 1 | Vehicle IP, 2X/week for 5 weeks | Vehicle | | Vehicle | Vehicle | | Vehicle |
| Group 2 | Docetaxel 10 mg/kg (IP, 1X/week | | DTX | | | DTX | |
| | for 5 weeks | | | | | | |
| Group 3 | Docetaxel 10 mg/kg (IP, 1X/week | AB16B5 | DTX | AB16B5 | AB16B5 | | AB16B5 |
| | for 2 weeks) and AB-16B5 10 | | | | | | |
| | mg/kg (IP, 2X/week for 5 weeks) | | | | | | |
| Group 4 | Docetaxel 10 mg/kg (IP, 1X/week | AB16B5 | DTX | AB16B5 | AB16B5 | DTX | AB16B5 |
| | for 5 weeks) and AB-16B5 10 | | | | | | |
| | mg/kg (IP, 2X/week for 5 weeks) | | | | | | |
| Group 5 | AB-16B5 10 mg/kg (IP, 2X/week | AB16B5 | | AB16B5 | AB16B5 | | AB16B5 |
| | for 5 weeks) | | | | | | |

*2 animals from Group 4 received docetaxel on the same day as AB16B5 (day 4) on week 3

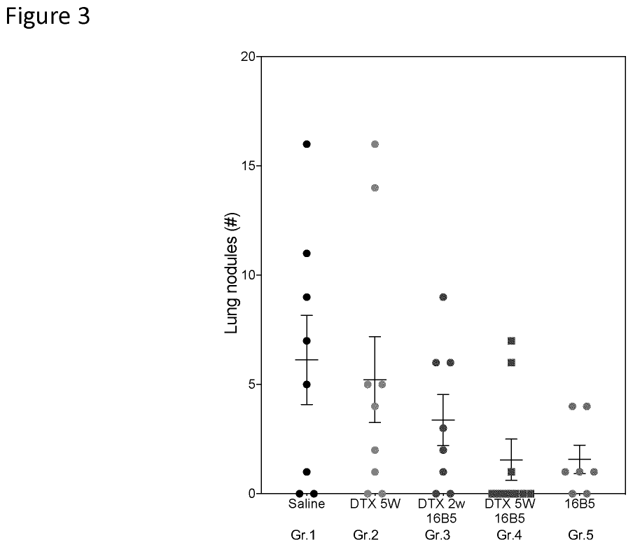

Results presented in FIG. 3, show that lungs of the animals from Group 4 and Group 5 comprised less metastatic lung nodules than the saline-control-treated mice. As well, mice that were treated in monotherapy with docetaxel had as many metastatic lung nodules as the saline control group. Treatment with docetaxel for two weeks in combination with 16B5 led to fewer metastatic lung nodules that in Group 1 and Group 2 but the response to treatment was not as extensive as in Group 4 and Group 5. There were more animals in Group 4 for which no nodules could be detected than in any other groups. These results suggest that AB-16B5 monotherapy or combination therapy with docetaxel effectively inhibit metastatic invasion in immunocompetent mice. These results also suggest that it may be preferable to administer AB-16B5 and docetaxel over the entire course of treatment.

The primary tumors excised at Day 16 post implantation, were processed with collagenase and hyaluronidase and immune cells were purified by positive selection using magnetic latex beads coated with an anti-CD45 antibody. The purified cells were transferred into small petri dishes containing culture medium supplemented with IL2 and IL7 to perform phenotypic analyses. It was found that very few CD45+ were present in the primary tumors retrieved from Group 1 and Group 2 animals. In contrast, there were more immune cells in tumors retrieved from Group 3, Group 4 and Group 5 animals.

Treatment of mice implanted with 4T1 tumor cells with docetaxel (DTX 5W) was relatively ineffective. The 4T1 tumors bear an EMT-high signature that causes resistance to many chemotherapeutic agents including docetaxel. Treatment of mice with docetaxel for 2 weeks and with 16B5 for 5 weeks was not as effective as treatment with 16B5 in monotherapy possibly because transient exposure of tumors to docetaxel resulted in increased resistance of tumors. The combination of docetaxel with 16B5 for 5 weeks proved to be the most effective therapeutic regimen. The combined increase of shed antigens caused by docetaxel and inhibition of EMT resulted in an increased immune response that translated in fewer lung metastases in this group compared to 16B5 in monotherapy.

AB-16B5 in monotherapy and the combination of AB-16B5 with docetaxel thus allow infiltration of immune cells in the tumor microenvironment in immunocompetent mice. These results also suggest that the primary tumors may be affected by the AB-16B5 monotherapy or combination therapy.

### Example 6—Characterization of Tumor Infiltrating Lymphocytes

Balb/c mice were orthotopically implanted with 5×10⁵ 4T1 cells in the 4th mammary fat pad. Animals received intraperitoneal (IP) AB-16B5 (murine 16B5) 10 mg/kg twice weekly in combination with IP docetaxel 10 mg/kg weekly (Group 15: animals 1501, 1502 and 1503) or IP AB-16B5 10 mg/kg twice weekly (Group 25: animal). The primary tumor was surgically removed at Day 16 post-implantation. The animals were sacrificed at Day 36 and the lungs were excised and each visible lung metastasis was carefully dissected. Each visible metastatic nodule, if any, was excised and processed for a rapid expansion of tumor infiltrating lymphocyte protocol. The metastatic nodules were sectioned into small pieces of 2-3 mm edge that were individually grown in 24 well plates containing culture medium supplemented with FBS, IL2, IL7, ITS (1,000 U/mL IL2, 2.0 ng/mL IL7 and 1× insulin-transferrin-selenium cocktail (Gibco 41400-045)).

After three weeks in culture, 100,000 cells were taken from each of the lymphocyte cultures (six cultures corresponding to three animals from Group 15 and three animals from Group 25) and placed in culture with 100,000 4T1 tumor cells. After overnight co-culture, the supernatant was recovered for INFγ quantification by ELISA.

The results of INFγ secretion from lymphocyte cultures in the presence of 4T1 cells indicate that lymphocytes isolated from lung metastatic nodules secrete INFγ at high levels with highest levels observed in the docetaxel-16B5 group (see Table 6). These results confirm that inhibition of EMT with the anti-sCLU 16B5 mAb contributes to the generation of a “warm” tumor microenvironment that allows the infiltration of T lymphocytes in tumors.

TABLE 6

| Sample | INFγ pg/mL |
|---|---|
| 1501 | 5370.0 |
| 1502 | 12488.8 |
| 1503 | 2326.3 |
| 2501 | 8538.8 |
| 2502 | 3770.0 |
| 2503 | 4538.8 |

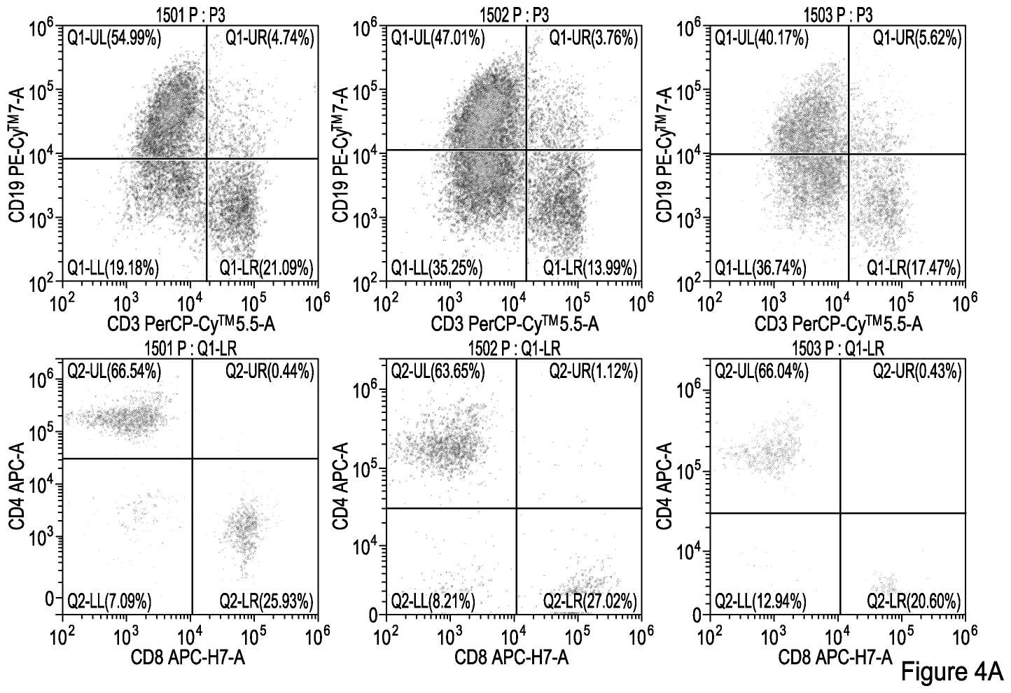

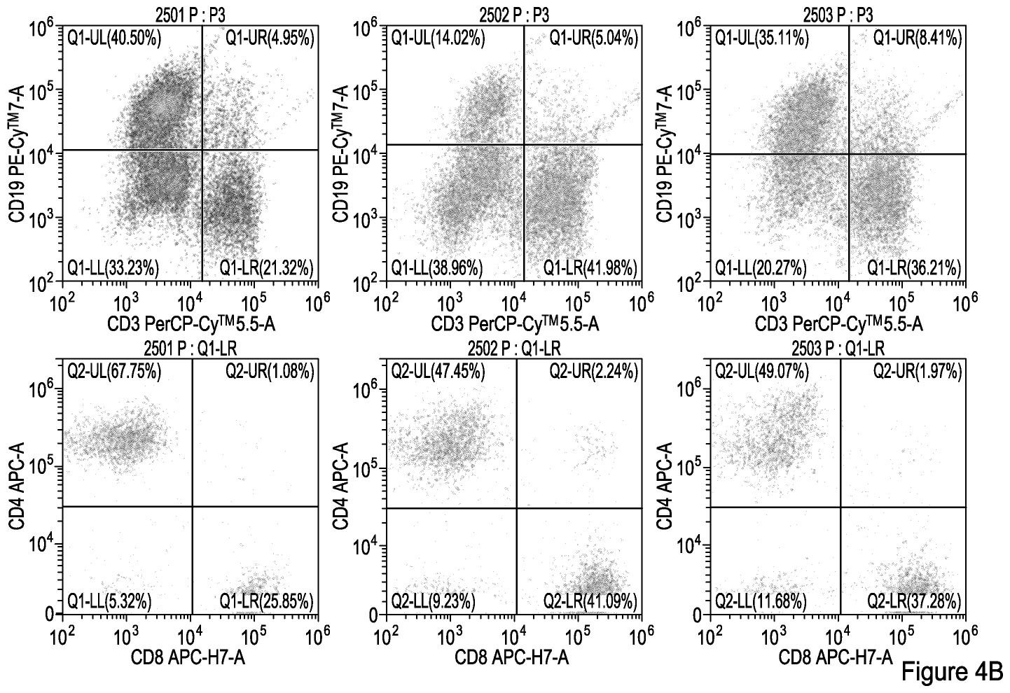

The lymphocytes were stimulated with anti-CD3 and anti-CD28 monoclonal antibodies. Lymphocytes from each donor animal were pooled and processed for flow cytometry analysis with antibodies against CD45 (lymphocyte common antigen), CD3, CD4, CD8 and CD19 (B-cell biomarker) (FIG. 4A and FIG. 4B). The resulting single cell preparations were initially selected on their size to select those corresponding to immune cells. They were further gated on an FSC/SSC plot to exclude dead cells and debris. Flow cytometric were then performed with antibodies against CD45, CD3, CD19, CD3, CD4 and CD8. Immune cells positive for CD45 were gated on CD3 and CD19 (P3). The CD3+ cells were further gated on CD4 and CD8 (Q1-LR).

Results indicated 80-90% cell viability of CD45+ cells for both groups. The CD45+ cells from group 15 (FIG. 4A) comprised 40.2% to 55.0% of CD19 cells and 14.0% to 21.1% of CD3+ cells. The CD3+ cells comprised 63.7% to 66.5% of CD4+ T cells and 20.6% to 27.0% CD8+ T cells. The CD45+ cells from group 25 (FIG. 4B) comprised 14.0% to 35.0% of CD19 cells and 21.3% to 42.0% of CD3+ cells. The CD3+ cells comprised 47.5% to 67.8% of CD4+ T cells and 25.9% to 41.1% CD8+ T cells.

Again, these results suggest that the primary tumors may be affected by the AB-16B5 monotherapy or combination therapy and that infiltration of tumor cells occur in both AB-16B5 monotherapy or combination therapy in immunocompetent mice.

Enhancing tumor T-cell infiltration with AB-16B5 may thus render tumors more susceptible to immunotherapy with checkpoint inhibitors or with cellular immunotherapy.

### Example 7—Phase II Clinical Study

The Applicant will evaluate the use of anti-clusterin antibodies combined with docetaxel in previously treated subjects with metastatic non-small cell lung cancer.

This Phase II study recruits 40 metastatic non-small cell lung cancer patients who failed treatment with a platinum-containing doublet treatment and an anti-PD1 or PDL-1 immune checkpoint antibody, administered simultaneously or sequentially. All recruited patients receive AB-16B5 (herein referred to as humanized 16B5) at a dose of 12 mg/kg once weekly combined with docetaxel at a dose of 75 mg/m² once every 3 weeks.

### Objectives

A primary objective of this study is to determine the objective response rate (ORR) per RECIST 1.1 in subjects receiving the combination of AB-16B5 and docetaxel.

Another primary objective of this study is to determine the objective response rate (ORR) per RECIST 1.1 in subjects receiving the combination of AB-16B5 and docetaxel.

Yet another primary objective of this study is to determine the safety and tolerability of the combination of AB-16B5 and docetaxel.

A secondary objective of this study is to determine the clinical benefit rate (complete response (CR), partial response (PR) and stable disease (SD)) per RECIST 1.1 in subjects receiving the combination of AB-16B5 and docetaxel.

Another secondary objective of this study is to determine the duration of response (CR and PR) per RECIST 1.1 in subjects receiving the combination of AB-16B5 and docetaxel.

Yet another secondary objective of this study is to determine the duration of stable disease per RECIST 1.1 in subjects receiving the combination of AB-16B5 and docetaxel.

A further secondary objective of this study is to determine the progression free survival (PFS) per RECIST 1.1 in subjects receiving the combination of AB-16B5 and docetaxel.

Another secondary objective of this study is to determine the overall survival (OS) in subjects receiving the combination of AB-16B5 and docetaxel.

Yet another secondary objective of this study is to determine the pharmacokinetics of AB-16B5 in this subject population.

An exploratory objective of this study is to perform exploratory pharmacodynamic evaluation of the effect of the combination of AB-16B5 and docetaxel on epithelial to mesenchymal transition (EMT) biomarkers, immune cell biomarkers and immune checkpoints in tumor biopsies.

An exploratory objective of this study is to evaluate disease response using iRECIST in subjects pursuing treatment beyond progression.

### Study Design

The study is an open-label, single-arm, multi-center Phase II trial of AB-16B5 in combination with docetaxel in previously treated subjects with metastatic non-small cell lung cancer who have experienced disease progression following treatment with a platinum-containing doublet treatment and an anti-PD1 or PDL-1 immune checkpoint antibody, administered simultaneously or sequentially. Approximately 40 subjects are enrolled in this trial and receive AB-16B5 at a dose of 12 mg/kg once weekly on Days 1, 8 and 15 combined with docetaxel at a dose of 75 mg/m² once every 3 weeks on Day 1. One cycle of treatment consists of 21 days (3 weeks). The safety profile of the AB-16B5 and docetaxel combination is examined during a safety lead-in period with the first 8 subjects completing one cycle of treatment.

Subjects are evaluated every 6 weeks with radiographic imaging to assess response to treatment using Response Evaluation Criteria in Solid Tumors (RECIST) 1.1 criteria for determination of the objective response rate (ORR) and progression free survival (PFS). A futility analysis is conducted to minimize subject exposure to an ineffective treatment. Paired tumor biopsies (pre-treatment and on-treatment) are collected in all subjects. Adverse events are monitored throughout the study and graded for severity according to the NCI Common Terminology Criteria for Adverse Events (CTCAE). Study treatment continues until there is evidence of disease progression (defined according to RECIST 1.1), treatment-related adverse events of unacceptable severity, subject request for discontinuation or Investigator determination that further treatment is not in the subject's best interest. Treatment beyond progression is allowed if the Investigator considers the subject to be clinically stable. Subjects who must discontinue docetaxel due to toxicity continue treatment with AB-16B5.

### Safety Lead-In Period

The safety profile of AB-16B5 at a dose of 12 mg/kg administered once weekly on Days 1, 8 and 15 combined with docetaxel at a dose of 75 mg/m² once every 3 weeks on Day 1 is examined during a safety lead-in period with the first 8 subjects completing one cycle of treatment. Decision to de-escalate the dose of AB-16B5 can be made using the modified toxicity probability interval method (mTPI)^{Error! Bookmark not defined}.

The study treatment is considered acceptable if no more than 3 dose limiting toxicity (DLTs) are observed during the first cycle in the first 8 subjects treated.

For these purposes, a DLT is defined as a Grade ≥3 non-hematologic toxicity occurring during Cycle 1 of therapy. In addition, the following hematologic toxicities are considered as a DLT:
Grade ≥4 neutropenia or thrombocytopenia >7 days
Grade ≥3 thrombocytopenia with bleeding
Grade ≥3 febrile neutropenia

Toxicities that are clearly and incontrovertibly due to disease progression or to extraneous causes are not to be considered DLTs. In addition, the following non-hematologic toxicities are not to be considered DLTs:
Grade 3 arthralgia or myalgia which returns to Grade ≤1 within <7 days with appropriate supportive care
Grade 3 nausea, vomiting or diarrhea which returns to Grade ≤1 within <72 hours with appropriate supportive care
Grade 3 fatigue lasting <7 days
Grade 3 electrolyte abnormalities that last less than 72 hours and are not associated with clinical symptoms
Grade 3 amylase or lipase elevation which is not associated with symptoms or clinical manifestations of pancreatitis

De-escalation of AB-16B5 is performed if more than 3 DLTs are observed in the first 8 subjects treated.

In such a case, the next three subjects are treated with AB-16B5 at 9 mg/kg AB-16B5 administered once weekly on Days 1, 8 and 15 combined with docetaxel at a dose of 75 mg/m² once every 3 weeks on Day 1. If 0 or 1 DLT is observed during the first cycle in these 3 subjects, AB-16B5 at the 9 mg/kg dose is considered acceptable.

If more than 1 DLTs are observed, a final de-escalation of AB-16B5 to 6 mg/kg is performed and the safety profile is evaluated using the same process as described above.

### Inclusion Criteria

Subjects enrolled in the study meet the following inclusion criteria:
Subjects (male or non-pregnant female) having >18 years of age on the day of signing the informed consent.
Subjects having a histologically or cytologically confirmed diagnosis of (Stage III-IV) non-small cell lung cancer (NSCLC) and with at least one measurable lesion defined by RECIST 1.1.
Subjects having experienced a disease progression following treatment with a an anti-PD1 or PDL-1 immune checkpoint antibody and a platinum-containing doublet treatment, administered simultaneously or sequentially.
Subjects having targetable driver mutation in EGFR or ALK gene are allowed on trial after failing available targeted therapies and having experienced a disease progression following treatment with an anti-PD1 or PDL-1 immune checkpoint antibody and a platinum-containing doublet treatment, administered simultaneously or sequentially.
Subjects having adequate organ and immune function as indicated in Table 7 below:

| | |
|---|---|
| System | Laboratory Value |

Hematological

| | |
|---|---|
| Absolute neutrophil count (ANC) | ≥1.5 × 10⁹/L (1,500 cells/mm³) |
| Platelets | ≥100 × 10⁹/L (100,000 cells/mm³ |
| Hemoglobin | ≥90 g/L |

Hepatic

| | |
|---|---|
| Total bilirubin | ≤ULN |
| AST (SGOT) and ALT (SGPT) | ≤1.5 × ULN, unless secondary |
| | to liver metastases |
| Alkaline phosphatase | ≤2.5 × ULN |

Renal

| | |
|---|---|
| Creatinine clearance^{a} | ≥60 mL/min |

Coagulation

| | |
|---|---|
| International Normalized Ratio (INR) | ≤1.5 × ULN^{b} |

^{a}Creatinine clearance should be calculated using the Cockcroft-Gault Method: CrCl = [(140-age) × weight (kg) × (0.85 for females only)]/(72 × serum creatinine)
^{b}if subject is receiving anticoagulant therapy, then INR must be within therapeutic range.

Subjects having a tumor lesion amenable for biopsies with no contraindication for biopsy.
Subjects having an Eastern Cooperative Oncology Group (ECOG) performance status of ≤2.
Subjects having a life expectancy of at least 3 months.
Subjects having recovered from the toxic effects resulting from the most recent cancer treatment to Grade 1 or less. If the subjects underwent major surgery or received radiation therapy, they have recovered from the complications and/or toxicity.
Female subjects of childbearing potential having a negative urine or serum pregnancy test within 72 hours prior to the first dose of study treatment. If the urine test is positive or cannot be confirmed as negative, a serum pregnancy test will be required. The serum pregnancy test should preferably be negative for the subject to be eligible.
Subjects (both male and female) of reproductive potential are willing to practice highly effective methods of contraception throughout the study and for up to 90 days after the last dose of study medication. Abstinence is acceptable if this is the subject's usual lifestyle.
Female subjects are not considered of childbearing potential if they have a history of surgical sterility or evidence of post-menopausal status defined as any of the following:
   ≥45 years of age and has not had menses for more than 2 years.
   Amenorrhoeic for <2 years without hysterectomy and oophorectomy and a follicle stimulating hormone (FSH) value in the postmenopausal range at screening.
   Post hysterectomy, oophorectomy or tubal ligation. Documented hysterectomy or oophorectomy must be confirmed with medical records of the actual procedure or by ultrasound. Tubal ligation must be confirmed with medical records of the actual procedure.

It is to be understood herein that the inclusion criteria are for the purpose of the clinical trial only and are not to be considered limitations of the approved drug for treatment.

### Exclusion Criteria

Subjects enrolled in the study meet the following exclusion criteria:
Subjects having received prior therapy with AB-16B5.
Subjects having received prior therapy with docetaxel for the treatment of NSCLC.
Subjects who are currently participating or has participated in a study of an investigational agent or using an investigational device within 21 days prior to the first dose of study treatment. The 21-day window should be calculated using the last dose of an antineoplastic investigational agent or last use of an investigational device with antineoplastic intent.
Subjects who have received any anti-cancer treatment within 3 weeks or radiation therapy within 2 weeks prior to receiving the first dose of study treatment or who have not recovered from adverse events to Grade 1 or less. Subjects with alopecia are eligible to participate.
Subjects who are expected to require any other form of systemic or localized antineoplastic therapy while on the trial. This includes maintenance therapy with another agent or radiation therapy.
Subjects who are receiving a dose >10 mg/day of prednisone (or equivalent) within 7 days prior to the first dose of study treatment or any other form of immunosuppressive medication (corticosteroid pre-treatment and/or post-treatment of docetaxel is allowed).
Subjects who require treatment with a strong CYP3A4 inhibitor (e.g., ketoconazole, itraconazole, clarithromycin, atazanavir, indinavir, nefazodone, nelfinavir, ritonavir, saquinavir, telithromycin and voriconazole). Subjects may be included if there is an alternate treatment with a weak CYP3A4 inhibitor and they are willing to change prior to randomization. If the subject agrees to change from a strong inhibitor to a weak CYP3A4 inhibitor, the strong inhibitor must be stopped at least 7 days prior to the first dose of study treatment.
Subjects who have another malignancy that is progressing or requires active treatment. Exceptions include basal cell carcinoma of the skin, squamous cell carcinoma of the skin or in situ cervical cancer.
Subjects who have known active central nervous system metastases and/or carcinomatous meningitis. Subjects with previously treated brain metastases may participate if they have been clinically stable for at least 2 weeks prior to the first dose of study treatment, if they have no evidence of new or enlarging brain metastases and if they are not receiving a dose >10 mg/day of prednisone (or equivalent) within 7 days prior to the first dose of study treatment.
Subjects with clinically significant ECG abnormalities.
Subjects who have received or will receive a live vaccine within 30 days prior to the first dose of study treatment.
Subjects with a known history of human immunodeficiency (HIV).
Subjects with an active Hepatitis B or C infection.
Subjects with an active infection requiring antibiotic therapy.
Subjects with a known history of alcohol or other substance abuse within the last year.
Subjects with known hypersensitivity to docetaxel or drugs formulated with polysorbate 80.
Subjects who have a history or current evidence of any condition, therapy or laboratory abnormalities that may confound the results of the trial, interfere with the subject's participation for the full duration of the trial or if it is not in the best interest of the subject to participate in the trial.
Subjects with medical, social or psychosocial factors that, in the opinion of the treating Investigator, could impact the safety or compliance with study procedures.
Subjects who are pregnant or lactating or who are expecting to conceive or father children within the projected duration of the trial through 90 days after the last dose of AB-16B5 or the last dose of docetaxel.

It is to be understood herein that the exclusion criteria are for the purpose of the clinical trial only and are not to be considered limitations of the approved drug for treatment.

### Study Treatment

### Study Drug, Dose and Mode of Administration

### AB-16B5

AB-16B5 is a humanized IgG₂ monoclonal antibody targeting sCLU for the inhibition of cancer-associated EMT (humanized 16B5). AB-16B5 is provided in 10 mL vials at a protein concentration of 10.0 mg/mL. AB-16B5 is formulated in a citrate buffer solution at pH 6.0.

AB-16B5 vials are stored upright at 2-8° C.

Subjects will receive AB-16B5 by a 60-minute IV infusion once weekly (refer to Pharmacy Manual for the infusion conditions) on Days 1 (prior to docetaxel infusion), 8 and 15. The dose of AB-16B5 will be determined during the safety lead-in period.

Subjects who experience infusion-related reactions will be treated with corticosteroids such as dexamethasone. Antihistamines and acetaminophen can also be used, as deemed appropriate.

Premedication to prevent infusion reaction related to AB-16B5 will not be employed initially. Subjects who have experienced infusion-related reactions will be premedicated as follows:
Grade 1 (mild): No premedication at subsequent dosing
Grade 2 (moderate): Dexamethasone 8 mg PO BID the day prior to AB-16B5 infusion and acetaminophen 650 mg PO and diphenhydramine 25-50 mg PO 30-60 minutes prior to AB-16B5 infusion
Grade 3 (severe) and Grade 4 (life-threatening): Subject will be discontinued from further treatment with AB-16B5

At any point during the study, if clinically significant infusion related reactions are observed in multiple subjects, the Investigator could decide in agreement with the Sponsor to implement a premedication for all new subjects.

### Docetaxel

Docetaxel will be administered at the dosage of 75 mg/m² by a 60-minute IV infusion once every 3 weeks on Day 1. Docetaxel will be prepared and administered as per the approved product label/monograph.

All subjects should be premedicated with corticosteroids as per hospital standard practices. Vein extravasation and accidental spillages should be dealt with according to the hospital standard practices.

### Treatment Duration

One cycle of treatment will consist of 21 days (3 weeks).

Study treatment will continue until there is evidence of disease progression, unacceptable toxicity, subject requests discontinuation of study treatment, or the Investigator feels that further treatment is not in the subject's best interest. Subjects who must discontinue docetaxel due to toxicity will continue on AB-16B5.

Treatment beyond progression will be allowed if the Investigator considers the subject to be clinically stable. The clinical judgment decision by the site should be based on the clinical stability of the subjects as defined below:

Clinical stability is defined as the following:
Absence of symptoms and signs indicating clinically significant progression of disease, including worsening of laboratory values
No decline in ECOG performance status
Absence of rapid progression of disease
Absence of progressive tumor at critical anatomical sites (e.g., cord compression) requiring urgent alternative medical intervention

Any subject deemed clinically unstable should be discontinued from trial treatment at first radiologic evidence of PD.

Study Treatment, Dose Reduction after Safety Lead-In

AB-16B5 Dose Reduction

Subjects who experience any Grade ≥3 adverse event that is judged to be possibly, probably or likely related to AB-16B5 and not requiring treatment discontinuation should have a reduction in AB-16B5 dose by one dose level (Table 8 below).

Treatment will be re-initiated at the lower dose only after recovery of the adverse event to Grade ≤1.

| Starting Dose Level | Reduced Dose |
|---|---|
| 12 mg/kg | 9 mg/kg |
| 9 mg/kg | 6 mg/kg |
| 6 mg/kg | Discontinue AB-16B5 |

Subjects whose original AB-16B5 dose has been reduced for toxicity will not be re-escalated.

### Docetaxel Dose Reduction

Subjects who experience either febrile neutropenia, neutrophils <500 cells/mm³ for more than one week, severe or cumulative cutaneous reactions, or other Grade ≥3 non-hematological toxicities that is judged to be related to docetaxel should have treatment withheld until resolution of the toxicity and then resumed at 60 mg/m². Based upon subject's condition, treatment with AB-16B5 may continue during this period.

Subjects who develop Grade ≥3 peripheral neuropathy should have docetaxel discontinued.

### Example 8—Phase II Clinical Study-Preliminary Results

The safety lead-in treatment period confirmed that a dose of AB-16B5 of 12 mg/kg once per week and a dose of docetaxel of 75 mg/m² is safe. It is thus expected that lower dosage could also be safe.

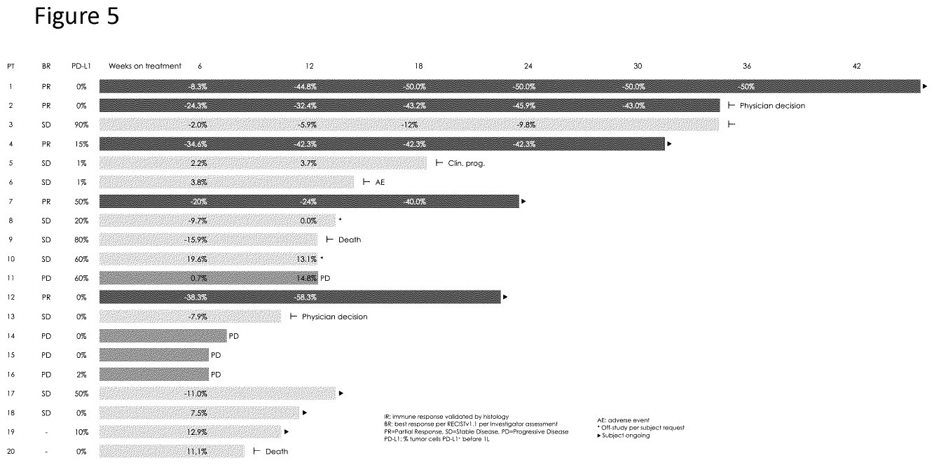

FIG. 5 is a chart showing the duration of patients on treatment as the best response as per RECIST 1.1 with 5 patients showing partial response (PR), 11 patients showing stable disease (SD) and 4 patients showing progressive diseases (PD) in 20 evaluable patients.

As can be seen from FIG. 5, 4 NSCLC patients out of 5 (Patient 1, 2, 4, and 12) showing evidence of partial response have low expression of PD-L1 ((TPS) of ≤15%).

Moreover, as can be seen from FIG. 5, in some patients, the clinical benefit is observed after only two cycles of treatment and lasts for at least 6 weeks and more after first determination of response to treatment.

Additionally, screening for KRAS mutation was performed in eight patients showing evidence of partial response or stable disease (Patient 3, 4, 6, 7, 8, 9, 12 and 13). Four were found to be negative for KRAS mutation (Patient 3, 4, 6 and 8) and four were positive (7, 9, 12 and 13) (data not shown).

Biopsies from some patients under treatment were obtained for analysis of various parameters. H&E stains were analyzed by a certified pathologist. Detections of tumor cell markers and immune cells were performed on successive histological sections. Slides were digitized and analyzed using algorithms created using open-source software QuPath™.

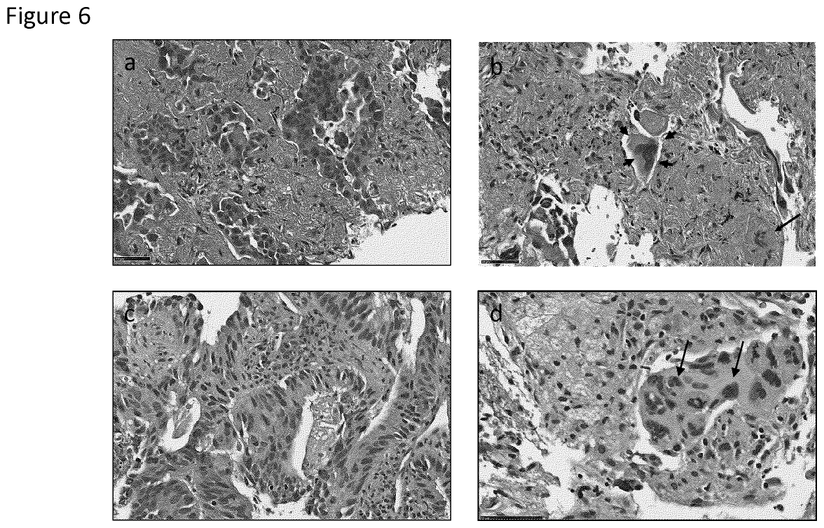

Histology analyses of pre- and on-treatment biopsies from two patients under study are illustrated in FIGS. 6a-6d. Tumor sections were stained with hematoxylin and eosin. The pre-treatment samples are largely composed of neoplastic tumor cells with no evidence of inflammatory cells (a and c). In contrast, very few tumor cells could be detected in the on-treatment samples (b and d). In addition, cytopathic changes caused by docetaxel were observed (arrows in b and d) as well as a brisk inflammatory infiltrate.

### Patient 1 (PT1)

The PD-L1 TPS score determined during the first-line therapy was 0%. Results from a scan after the eight cycles of treatment indicated a 50% decrease in the size of the target lesions. This patient therefore shows evidence of partial response.

Analysis of the pre-treatment biopsy revealed a modest lymphocytic infiltration in one specimen while fibrosis and a chronic inflammatory infiltrate was present in another one. However, the largest portion of another core was replaced by neoplastic tumor cells with massive necrosis. No residual tumor cells could be found on any of the four cores from the on-treatment biopsy. Fibrotic tissue with chronic lymphoplasmacytic inflammation was observed in on-treatment tissue cores. Lymphocytes and plasma cells infiltration was found to be pronounced in certain areas. On-treatment samples fail to demonstrate residual tumor cells. Of note, fibrosis and chronic inflammation are present in on-treatment cores. Tumor cells were noted only in pre-treatment conditions.

Analysis of the Ki-67 proliferation biomarker indicated a dense staining in the pre-treatment condition and a strong reduction of the signal in the on-treatment samples. Image analyses revealed a decrease of 42% of proliferative tumor cells after the second cycle of treatment.

Image analysis of E-cadherin staining in the pre-treatment biopsy revealed a large number of tumor cells with an H-score of 166. No E-cadherin staining could be observed in the on-treatment biopsy suggesting that the treatment was effective in eradicating the tumor cells.

Image analysis of CD3 staining in the pre-treatment biopsy revealed that the tumor was poorly infiltrated with T lymphocytes before initiation of treatment. However, a 45% increase of CD3 signal was observed in the on-treatment biopsy.

Image analysis of CD4 staining showed an 100% increase of CD4 T cells in the on-treatment biopsy which increased from 4% to 8% of cells in the region of interest.

Image analysis of CD8 staining showed an increase of 86% of CD8 T cells in the on-treatment biopsy which increased from 7% to 13% of cells in the region of interest.

### Patient 2 (PT2)

The PD-L1 TPS score determined during the first-line therapy was 0%. The patient has stopped receiving docetaxel at week 33. CT scans were performed after the second, fourth, sixth, eighth and tenth cycle indicating a decrease of the size of the target lesions from 24% at the end the cycle 2 to 43% at the end of cycle 10. This patient therefore shows evidence of partial response.

Analysis of tumor biopsies revealed that pre-treatment samples are more basophilic reflecting the presence of numerous tumor cells aggregates. In contrast, samples from the on-treatment (end of cycle 2) showed large eosinophilic areas reflecting extensive stromal fibrosis. Higher power view of pre-treatment condition showed widespread tumor involvement. Of note, tumor nuclei display cytological abnormalities but no alteration to suggest recent exposure to chemotherapy. There was no inflammatory reaction surrounding neoplastic foci. In on-treatment condition, important findings were reported: cytopathic effects, fibrosis and chronic lymphoplasmacytic and histiocytic inflammation. There were areas of dense sclerotic tissue with chronic inflammatory cells and few residual tumor cells with cytopathic changes consistent with chemotherapeutic agent acting on cellular spindle. Large aneuploid and giant cells are reminiscent of changes induced by docetaxel.

Analysis of the Ki-67 proliferation biomarker indicated a dense staining in the pre-treatment condition and a strong reduction of the signal in the on-treatment samples. Image analyses revealed a decrease of 67.2% of proliferative cells after the second cycle of treatment. Image analysis of E-cadherin staining in the pre-treatment biopsy revealed a large number of tumor cells with an H-score of 166. No E-cadherin staining could be observed in the on-treatment biopsy suggesting that the treatment was effective in eradicating the tumor cells.

Image analysis of CD3 staining in the pre-treatment biopsy revealed that the tumor was poorly infiltrated with T lymphocytes before initiation of treatment. However, a 304% increase of CD3 signal was observed in the on-treatment biopsy.

Image analysis of CD4 staining showed an 100% increase of CD4 T cells in the on-treatment biopsy which increased from 4% to 8% of cells in the region of interest.

Image analysis of CD8 staining showed an increase of 86% of CD8 T cells in the on-treatment biopsy which increased from 7% to 13% of cells in the region of interest.

### Patient 3 (PT3)

The PD-L1 TPS score determined during the first-line therapy was 90%. The patient remained on first-line treatment for 14 months until tumor progression. Results from CT-scan after the sixth cycle of treatment indicated a 12% decrease in the size of the tumor lesions. This patient therefore shows evidence of stable disease.

### Patient 4 (PT4)

The PD-L1 TPS score determined during the first-line therapy was 15%. Results from CT-scan indicated a 34.6% and 42% decrease in the size of the tumor lesions after two and four cycles of treatment respectively. This patient therefore shows evidence of partial response.

In summary, tumor biopsies obtained prior to the anti-clusterin antibody and docetaxel combination treatment revealed that the tumor was poorly infiltrated with T lymphocytes. In contrast, on-treatment biopsies showed evidence of lymphoplasmacytic and/or histiocytic inflammation. The presence of tumor infiltrating lymphocytes (TILs), tertiary lymphoid structures (TLSs), macrophages and NK cells was particularly observed.

Records also show that some of the subjects enrolled in this clinical trial had tumors with low PD-L1 expression or with no evidence of PD-L1 expression prior to immune checkpoint antibody treatment. Nine out of fifteen patients have a TPS of ≤15%. Out of these nine patients, seven are characterized has having a TPS of ≤1% and five may be further characterized as having a TPS of <1%.

The relatively poor immune cell infiltration observed in tumor biopsies combined with low expression of PD-L1 may explain in part, failure of treatment comprising an anti-PD-1 or anti-PD-L1 immune checkpoint antibody.

Preliminary results of NCT04364620 indicate that the best response as per Response Evaluation Criteria in Solid Tumors (RECIST) 1.1 criteria is partial response (PR) for 5 patients, stable disease (SD) for 11 patients and progressive diseases (PD) for 4 patients for a total of 20 evaluable patients (For RECIST 1.1 criteria see Eisenhauer, Eur J Cancer, 45 (2009) 228-247). These preliminary data suggest signs of efficacy. Moreover, current data suggest an increase in progression-free survival compared to single-agent docetaxel in the same patient population.

It also appears that NSCLC patients with low PD-L1 tumor proportion score (TPS) may benefit from the treatment of the present disclosure.

For example, evidence of partial response (as defined by RECIST 1.1) was found for four NSCLC patients having a tumor with a PD-L1 tumor proportion score (TPS) of ≤15%. Three of these patients have a PD-L1 TPS of <1% including one patient having a KRAS mutation.

Evidence of stable disease as defined by RECIST 1.1 has also been observed in three NSCLC patient having a tumor with a PD-L1 tumor proportion score (TPS) of ≤1%, including one patient having a tumor with a PD-L1 tumor proportion score (TPS) of <1% carrying a KRAS mutation.

The anti-cancer therapy of the present disclosure may represent an alternative to anti-PD-1 or anti-PD-L1 immune checkpoint monotherapy or in combination with chemotherapy especially in patients that carries lesions with low expression of PD-L1 or with no evidence of PD-L1 expression. The anti-cancer therapy of the present disclosure may also represent an alternative for cancer patients that are not eligible for or would unlikely benefit from treatment comprising a PD-1 or PD-L1 immune checkpoint inhibitor.

The data presented herein show that the anti-cancer treatment of the present disclosure is safe and show signs of efficacy.

The data presented herein also show that the anti-cancer treatment of the present disclosure promotes infiltration of immune cells. The treatment may in turn lead to regression of lesions or stabilization of their growth. The anti-cancer therapy of the present disclosure may thus be used to modulate the antitumor immune response.

The embodiments and examples described herein are illustrative and are not meant to limit the scope of the claims. Variations of the foregoing embodiments, including alternatives, modifications and equivalents, are intended by the inventors to be encompassed by the claims. Citations listed in the present application are incorporated herein by reference.

### REFERENCES

Al-Lazikani et al., Standard conformations for the canonical structures of immunoglobulins. J Mol Biol 273:927-948, 1997.
Brochet et al. IMGT/V-QUEST: the highly customized and integrated system for IG and TR standardized V-J and V-D-J sequence analysis. Nucl Acids Res 36:W503-W508, 2008.
Andrew C. R. Martin, Antibody Engineering Vol. 2, Chapter 3: Protein Sequence and Structure Analysis of Antibody Variable Domains. R. Kontermann and S. Dubel (eds.), DOI 10.1007/978-3-642-01147-4_3, #Springer-Verlag Berlin Heidelberg 2010
Shibue, T., Weinberg, R. EMT, CSCs, and drug resistance: the mechanistic link and clinical implications. Nat Rev Clin Oncol 14: 611-629 (2017).
Terry, S., Savagner, P., Ortiz-Cuaran, S., Mahjoubi, L., Saintigny, P., Thiery, J.-P. and Chouaib, S., New insights into the role of EMT in tumor immune escape. Mol Oncol, 11: 824-846 (2017).
Lenferink, A., Cantin, C., Nantel, A. et al. Transcriptome profiling of a TGF-β-induced epithelial-to-mesenchymal transition reveals extracellular clusterin as a target for therapeutic antibodies. Oncogene 29: 831-844 (2010).
New response evaluation criteria in solid tumours: Revised RECIST guideline (version 1.1)” E. A. Eisenhauer, P. Therasse, J. Bogaerts, L. H. Schwartz, D. Sargent, R. Ford, J. Dancey, S. Arbuck, S. Gwyther, M. Mooney, L. Rubinstein, L. Shankar, L. Dodd, R. Kaplan, D. Lacombe, J. Verweij: Eur J Cancer, 45 (2009) 228-24.
Cristiano Ferrario, Julie Laurin, Leon Van Kempen, Caroline Lambert, Alan Spatz, Oksana Markova, Gerald Batist, Adrian Langleben, Mario Filion, Jacques Jolivet. Phase 1 first-in-human study of anti-clusterin antibody AB-16B5 in patients with advanced solid malignancies [abstract]. In: Proceedings of the American Association for Cancer Research Annual Meeting 2017: 2017 Apr. 1-5: Washington, DC. Philadelphia (PA): AACR: Cancer Res 2017:77(13 Suppl): Abstract nr CT098. doi: 10.1158/1538-7445.AM2017-CT098.
Hodge, J. W. Garnett, C. T., Farsaci, B., et al. Chemotherapy-induced immunogenic modulation of tumor cells enhances killing by cytotoxic T lymphocytes and is distinct from immunogenic cell death. Int. J. Cancer. 133: 624-636 (2013).
Jiang X, Dudzinski S, Beckermann K E, et al. MRI of tumor T cell infiltration in response to checkpoint inhibitor therapy. Journal for ImmunoTherapy of Cancer 2020:8:e000328. Doi: 10.1136/jitc-2019-000328.
MacCallum, R. M., Martin, A. C. R. and Thornton, J. T. ‘Antibody-antigen interactions: Contact analysis and binding site topography J. Mol. Biol. 262:732-745, 1996.
Wu and Kabat, An analysis of the sequences of the variable regions of Bence Jones proteins and myeloma light chains and their implications for antibody complementarity. J Exp Med 132:211-250, 1993.

TABLE 9
SEQUENCE LISTING TABLE

| | | SEQ ID |
|---|---|---|
| ID | SEQUENCE | NO: |
| 16B5 CDRL1 | KSSQSLLNSRTRKNYLA | 1 |
| 16B5 CDRL2 | WASTRES | 2 |
| 16B5 CDRL3 | KQSYNLWT | 3 |
| 16B5 CDRH1_v1 | GFNIKDIYMH | 4 |
| 16B5 CDRH2_v1 | RIDPAYGNTKYDPKFQG | 5 |
| 16B5 CDRH3_v1 | RYDTAMDY | 6 |
| 16B5 CDRH1_v2 | GFNIKDIY | 35 |
| 16B5 CDRH2_v2 | IDPAYGNT | 36 |
| 16B5 CDRH3_v2 | ARRYDTAMDY | 37 |
| murine 16B5 VL | DIVMSQSPSSLAVSAGEKVTMSCKSSQSLLNSRTRKN | 7 |
| | YLAWYQQKPGQSPKLLIYWASTRESGVPDRFTGSGSG | |
| | TDFTLTISSVQAEDLAVYYCKQSYNLWTFGGGTKLEF | |
| | K | |
| murine 16B5 VH | EVQLQQSGAELVKPGASVRLSCTTSGFNIKDIYMHWV | 8 |
| | KQRPEQGLEWIGRIDPAYGNTKYDPKFQGKATITADT | |
| | SSNTAYLQLSSLTSEDTAVYYCARRYDTAMDYWGQG | |
| | TSVTVSS | |
| Humanized 16B5 | DIVMTQSPDSLAVSLGERATINCKSSQSLLNSRTRKNY | 9 |
| VL | LAWYQQKPGQPPKLLIYWASTRESGVPDRFSGSGSGT | |
| | DFTLTISSLQAEDVAVYYCKQSYNLWTFGQGTKLEIK | |
| Humanized 16B5 | QVQLVQSGAEVKKPGATVKISCKVSGFNIKDIYMHW | 10 |
| VH | VQQAPGKGLEWMGRIDPAYGNTKYDPKFQGRVTITA | |
| | DTSTDTAYMELSSLRSEDTAVYYCARRYDTAMDYWG | |
| | QGTLVTVSS | |
| Humanized 16B5 | DIVMTQSPDSLAVSLGERATINCKSSQSLLNSRTRKNYL | 11 |
| light chain | AWYQQKPGQPPKLLIYWASTRESGVPDRFSGSGSGTDF | |
| | TLTISSLQAEDVAVYYCKQSYNLWTFGQGTKLEIKVAA | |
| | PSVFIFPPSDEQLKSGTASVVCLLNNFYPREAKVQWKV | |
| | DNALQSGNSQESVTEQDSKDSTYSLSSTLTLSKADYEK | |
| | HKVYACEVTHQGLSSPVTKSFNRGEC | |
| Humanized 16B5 | QVQLVQSGAEVKKPGATVKISCKVSGFNIKDIYMHWV | 12 |
| heavy chain | QQAPGKGLEWMGRIDPAYGNTKYDPKFQGRVTITADT | |
| | STDTAYMELSSLRSEDTAVYYCArRYDTAMDYwgqgtlvt | |
| | VSSASTKGPSVFPLAPCSRSTSESTAALGCLVKDYFPEPV | |
| | TVSWNSGALTSGVHTFPAVLQSSGLYSLSSVVTVPSSN | |
| | FGTQTYTCNVDHKPSNTKVDKTVERKCCVECPPCPAPP | |
| | VAGPSVFLFPPKPKDTLMISRTPEVTCVVVDVSHEDPE | |
| | VQFNWYVDGVEVHNAKTKPREEQFNSTFRVVSVLTVV | |
| | HQDWLNGKEYKCKVSNKGLPAPIEKTISKTKGQPREPQ | |
| | VYTLPPSREEMTKNQVSLTCLVKGFYPSDIAVEWESNG | |
| | QPENNYKTTPPMLDSDGSFFLYSKLTVDKSRWQQGNV | |
| | FSCSVMHEALHNHYTQKSLSLSPGK | |
| Humanized16B5 | ATGGTGCTGCAGACCCAGGTGTTCATCTCCCTGCTGC | 13 |
| light chain | TGTGGATCTCCGGCGCCTACGGCGACATCGTGATGA | |
| nucleic acid | CCCAGTCCCCCGACTCCCTGGCCGTGTCCCTGGGCGA | |
| | GAGAGCCACCATCAACTGCAAGTCCTCCCAGTCCCT | |
| | GCTGAACTCCCGGACCCGGAAGAACTACCTGGCCTG | |
| | GTATCAGCAGAAGCCTGGCCAGCCTCCTAAGCTGCT | |
| | GATCTACTGGGCCTCCACCCGGGAGTCCGGCGTGCC | |
| | TGACCGGTTCTCCGGCTCCGGCAGCGGCACCGACTT | |
| | CACCCTGACCATCAGCTCCCTGCAGGCCGAGGACGT | |
| | GGCCGTGTACTACTGCAAGCAGTCCTACAACCTGTG | |
| | GACCTTCGGCCAGGGCACCAAGCTGGAGATCAAGCG | |
| | GACTGTGGCTGCACCATCTGTCTTCATCTTCCCGCCA | |
| | TCTGATGAGCAGTTGAAATCTGGAACTGCCTCTGTTG | |
| | TGTGCCTGCTGAATAACTTCTATCCCAGAGAGGCCA | |
| | AAGTACAGTGGAAGGTGGATAACGCCCTCCAATCGG | |
| | GTAACTCCCAGGAGAGTGTCACAGAGCAGGACAGCA | |
| | AGGACAGCACCTACAGCCTCAGCAGCACCCTGACGC | |
| | TGAGCAAAGCAGACTACGAGAAACACAAAGTCTAC | |
| | GCCTGCGAAGTCACCCATCAGGGCCTGAGCTCGCCC | |
| | GTCACAAAGAGCTTCAACAGGGGAGAGTGTTAG | |
| Humanized 16B5 | ATGGACTGGACCTGGCGGATCCTGTTCCTGGTGGCC | 14 |
| heavy chain | GCTGCTACCGGCACCCACGCCCAGGTGCAGCTGGTG | |
| nucleic acid | CAGTCTGGCGCCGAGGTGAAGAAGCCTGGCGCCACC | |
| | GTCAAGATCAGCTGCAAGGTGTCCGGCTTCAACATC | |
| | AAGGACATCTACATGCACTGGGTGCAGCAGGCTCCA | |
| | GGCAAGGGACTGGAGTGGATGGGCCGGATCGACCCT | |
| | GCCTACGGCAACACCAAGTACGACCCTAAGTTCCAG | |
| | GGCCGGGTGACCATCACCGCCGACACCTCCACCGAC | |
| | ACCGCCTACATGGAACTGTCCTCCCTGCGGTCCGAG | |
| | GACACCGCCGTGTACTACTGCGCCCGGAGATACGAC | |
| | ACCGCCATGGATTACTGGGGCCAGGGCACCCTGGTG | |
| | ACCGTGTCCTCCGCTTCCACCAAGGGCCCATCGGTCT | |
| | TCCCCCTGGCGCCCTGCTCCAGGAGCACCTCCGAGA | |
| | GCACAGCGGCCCTGGGCTGCCTGGTCAAGGACTACT | |
| | TCCCCGAACCGGTGACGGTGTCGTGGAACTCAGGCG | |
| | CTCTGACCAGCGGCGTGCACACCTTCCCAGCTGTCCT | |
| | ACAGTCCTCAGGACTCTACTCCCTCAGCAGCGTGGT | |
| | GACCGTGCCCTCCAGCAACTTCGGCACCCAGACCTA | |
| | CACCTGCAACGTAGATCACAAGCCCAGCAACACCAA | |
| | GGTGGACAAGACAGTTGAGCGCAAATGTTGTGTCGA | |
| | GTGCCCACCGTGCCCAGCACCACCTGTGGCAGGACC | |
| | GTCAGTCTTCCTCTTCCCCCCAAAACCCAAGGACACC | |
| | CTCATGATCTCCCGGACCCCTGAGGTCACGTGCGTG | |
| | GTGGTGGACGTGAGCCACGAAGACCCCGAGGTCCAG | |
| | TTCAACTGGTACGTGGACGGCGTGGAGGTGCATAAT | |
| | GCCAAGACAAAGCCACGGGAGGAGCAGTTCAACAG | |
| | CACGTTCCGTGTGGTCAGCGTCCTCACCGTTGTGCAC | |
| | CAGGACTGGCTGAACGGCAAGGAGTACAAGTGCAA | |
| | GGTCTCCAACAAAGGCCTCCCAGCCCCCATCGAGAA | |
| | AACCATCTCCAAAACCAAAGGGCAGCCCCGAGAACC | |
| | ACAGGTGTACACCCTGCCCCCATCCCGGGAGGAGAT | |
| | GACCAAGAACCAGGTCAGCCTGACCTGCCTGGTCAA | |
| | AGGCTTCTACCCCAGCGACATCGCCGTGGAGTGGGA | |
| | GAGCAATGGGCAGCCGGAGAACAACTACAAGACCA | |
| | CACCTCCCATGCTGGACTCCGACGGCTCCTTCTTCCT | |
| | CTACAGCAAGCTCACCGTGGACAAGAGCAGGTGGCA | |
| | GCAGGGGAACGTCTTCTCATGCTCCGTGATGCATGA | |
| | GGCTCTGCACAACCACTACACGCAGAAGAGCCTCTC | |
| | CCTGTCTCCGGGTAAATGA | |
| 21B12 CDRL1 | KSSQSLLYSSNQKNYLA | 15 |
| 21B12 CDRL2 | WASTRES | 16 |
| 21B12 CDRL3 | QQYYIYPRT | 17 |
| 21B12 | GYTFTNYGMH | 18 |
| CDRH1_v1 | | |
| 21B12 | WINTYTGEPTYADDFKG | 19 |
| CDRH2_v1 | | |
| 21B12 | DGFLYFFDY | 20 |
| CDRH3_v1 | | |
| 21B12 | GYTFTNYG | 38 |
| CDRH1_v2 | | |
| 21B12 | INTYTGEP | 39 |
| CDRH2_v2 | | |
| 21B12 | DGFLYFFDY | 40 |
| CDRH3_v2 | | |
| Murine 21B12 | DIVMSQSPSSLAVSVGEKVTMSCKSSQSLLYSSNQKNY | 21 |
| VL | LAWYQQRPGQSPKLLIYWASTRESGVPDRFTGSGSGTD | |
| | FTLTISSVKAEDLAVYYCQQYYIYPRTFGGGTKLEIK | |
| Murine 21B12 | QIQLVQSGPELKKPGETVKISCKASGYTFTNYGMHWV | 22 |
| VH | KQAPGKGLKWMGWINTYTGEPTYADDFKGRFAFSLET | |
| | SASTAYLQINNLKNEDTATYFCARDGFLYFFDYWGQG | |
| | TTLTVSS | |
| Humanized | DIVMTQSPDSLAVSLGERATINCKSSQSLLYSSNQKNYL | 23 |
| 21B12 VL | AWYQQKPGQPPKLLIYWASTRESGVPDRFSGSGSGTDF | |
| | TLTISSLQAEDVAVYYCQQYYIYPRTFGQGTKLEIK | |
| Humanized | QVQLVQSGSELKKPGASVKVSCKASGYTFTNYGMHW | 24 |
| 21B12 VH | VRQAPGQGLEWMGWINTYTGEPTYADDFKGRFVFSLD | |
| | TSVSTAYLQISSLKAEDTAVYYCARDGFLYFFDYWGQ | |
| | GTLVTVSS | |
| Humanized | DIVMTQSPDSLAVSLGERATINCKSSQSLLYSSNQKNYL | 25 |
| 21B12 light chain | AWYQQKPGQPPKLLIYWASTRESGVPDRFSGSGSGTDF | |
| | TLTISSLQAEDVAVYYCQQYYIYPRTFGQGTKLEIKRTV | |
| | AAPSVFIFPPSDEQLKSGTASVVCLLNNFYPREAKVQW | |
| | KVDNALQSGNSQESVTEQDSKDSTYSLSSTLTLSKADY | |
| | EKHKVYACEVTHQGLSSPVTKSFNRGEC | |
| Humanized | QVQLVQSGSELKKPGASVKVSCKASGYTFTNYGMHW | 26 |
| 21B12 heavy | VRQAPGQGLEWMGWINTYTGEPTYADDFKGRFVFSLD | |
| chain | TSVSTAYLQISSLKAEDTAVYYCARdGFLYFFDYWGQG | |
| | TLVTVSSASTKGPSVFPLAPCSRSTSESTAALGCLVKDY | |
| | FPEPVTVSWNSGALTSGVHTFPAVLQSSGLYSLSSVVT | |
| | VPSSNFGTQTYTCNVDHKPSNTKVDKTVERKCCVECPP | |
| | CPAPPVAGPSVFLFPPKPKDTLMISRTPEVTCVVVDVSH | |
| | EDPEVQFNWYVDGVEVHNAKTKPREEQFNSTFRVVSV | |
| | LTVVHQDWLNGKEYKCKVSNKGLPAPIEKTISKTKGQ | |
| | PREPQVYTLPPSREEMTKNQVSLTCLVKGFYPSDIAVE | |
| | WESNGQPENNYKTTPPMLDSDGSFFLYSKLTVDKSRW | |
| | QQGNVFSCSVMHEALHNHYTQKSLSLSPGK | |
| Humanized | ATGGTGCTGCAGACCCAGGTGTTCATCTCCCTGCTGC | 27 |
| 21B12 light chain | TGTGGATCTCTGGCGCCTACGGCGACATCGTGATGA | |
| nucleic acid | CCCAGTCCCCCGACTCTCTGGCTGTGTCCCTGGGCGA | |
| | GCGGGCCACCATCAACTGCAAGTCCTCCCAGTCCCT | |
| | GCTGTACTCCTCCAACCAGAAGAACTACCTGGCCTG | |
| | GTATCAGCAGAAGCCTGGCCAGCCTCCTAAGCTGCT | |
| | GATCTACTGGGCCTCCACCCGGGAATCTGGCGTGCC | |
| | TGACCGGTTCTCCGGCTCTGGCTCCGGCACCGACTTC | |
| | ACCCTGACCATCAGCTCCCTGCAGGCCGAGGACGTG | |
| | GCCGTGTACTACTGCCAGCAGTACTACATCTACCCTC | |
| | GGACCTTCGGCCAGGGCACCAAGCTGGAAATCAAGC | |
| | GGACCGTGGCCGCTCCTTCCGTGTTCATCTTCCCCCC | |
| | TTCCGACGAGCAGCTGAAGTCCGGCACCGCCTCTGT | |
| | GGTGTGCCTGCTGAACAACTTCTACCCCCGGGAGGC | |
| | CAAGGTGCAGTGGAAGGTGGACAACGCCCTGCAGTC | |
| | CGGCAACTCCCAGGAATCCGTCACCGAGCAGGACTC | |
| | CAAGGACTCTACCTACTCCCTGTCCTCCACCCTGACC | |
| | CTGTCCAAGGCCGACTACGAGAAGCACAAGGTGTAC | |
| | GCCTGCGAAGTGACCCACCAGGGCCTGTCCTCTCCC | |
| | GTGACCAAGTCCTTCAACCGGGGCGAGTGCTGA | |
| Humanized | ATGGACTGGACCTGGCGGATCCTGTTTCTGGTGGCC | 28 |
| 21B12 heavy | GCTGCTACCGGCACACACGCCCAGGTGCAGCTGGTG | |
| chain nucleic acid | CAGTCCGGCTCCGAGCTGAAGAAACCTGGCGCCTCC | |
| | GTGAAGGTGTCCTGCAAGGCCTCCGGCTACACCTTC | |
| | ACCAACTACGGCATGCACTGGGTGCGCCAGGCACCT | |
| | GGACAGGGACTGGAATGGATGGGCTGGATCAACACC | |
| | TACACCGGCGAGCCTACCTACGCCGACGACTTCAAG | |
| | GGCAGATTCGTGTTCTCCCTGGACACCTCCGTGTCCA | |
| | CCGCCTACCTGCAGATCTCCTCCCTGAAGGCCGAGG | |
| | ACACCGCCGTGTACTACTGCGCCAGGGACGGCTTCC | |
| | TGTACTTCTTCGACTACTGGGGCCAGGGCACCCTGGT | |
| | GACCGTGTCCTCTGCCTCCACCAAGGGCCCTTCCGTG | |
| | TTCCCTCTGGCCCCTTGCTCCCGGTCCACCTCTGAGT | |
| | CTACCGCCGCTCTGGGCTGCCTGGTGAAGGACTACTT | |
| | CCCTGAGCCTGTGACAGTGTCCTGGAACTCTGGCGC | |
| | CCTGACCTCTGGCGTGCACACCTTCCCTGCCGTGCTG | |
| | CAGTCCTCCGGCCTGTACTCCCTGTCCTCCGTGGTGA | |
| | CAGTGCCTTCCTCCAACTTCGGCACCCAGACCTACAC | |
| | CTGCAACGTGGACCACAAGCCTTCCAACACCAAGGT | |
| | GGACAAGACCGTGGAGCGGAAGTGCTGCGTGGAGT | |
| | GCCCTCCTTGTCCTGCTCCTCCTGTGGCTGGCCCTAG | |
| | CGTGTTCCTGTTCCCTCCTAAGCCTAAGGACACCCTG | |
| | ATGATCTCCCGGACCCCTGAAGTGACCTGCGTGGTG | |
| | GTGGACGTGTCCCACGAGGACCCTGAGGTGCAGTTC | |
| | AATTGGTACGTGGACGGCGTGGAGGTGCACAACGCC | |
| | AAGACCAAGCCTCGGGAGGAACAGTTCAACTCCACC | |
| | TTCCGGGTGGTGTCCGTGCTGACCGTGGTGCACCAG | |
| | GACTGGCTGAACGGCAAAGAATACAAGTGCAAGGT | |
| | GTCCAACAAGGGCCTGCCTGCCCCTATCGAAAAGAC | |
| | CATCTCTAAGACCAAGGGCCAGCCTCGCGAGCCTCA | |
| | GGTGTACACCCTGCCTCCCTCCCGCGAGGAAATGAC | |
| | CAAGAACCAGGTGTCCCTGACCTGTCTGGTGAAGGG | |
| | CTTCTACCCTTCCGATATCGCCGTGGAGTGGGAGTCT | |
| | AACGGCCAGCCTGAGAACAACTACAAGACCACCCCT | |
| | CCTATGCTGGACTCCGACGGCTCCTTCTTCCTGTACA | |
| | GCAAGCTGACAGTGGACAAGTCCCGGTGGCAGCAGG | |
| | GCAACGTGTTCTCCTGCTCCGTGATGCACGAGGCCCT | |
| | GCACAACCACTACACCCAGAAGTCCCTGTCCCTGTCT | |
| | CCTGGCAAGTGA | |
| 20E11 variable | DIVLTLSPASLAVSLGQRATISCRASQSVNSSNYSYMH | 29 |
| light chain | WYQQKPGQPPKLLIKYASNLESGVPARFSGSGSGTHFT | |
| | LNIHPVEEEDTATYYCQHSWEIPWTFGGGTKLEIK | |
| 20E11 variable | QIQLVQSGPELKKPGETVKISCKASGYTFTDYSMHWVK | 30 |
| heavy chain | QAPGKGLKWMGWINTETGEPTYADDFKGRFAFSLETS | |
| | ASTAYLQINNLKNEDTATYFCARTGSSGYFDCWGQGT | |
| | TLTVSS | |
| 11E2 variable | ENVLTQSPAIMSASPGEKVTMTCSASSSVSYMHWYQQ | 31 |
| light chain Gr1 | KSSTSPKLWIYDTSKLASGVPGRFSGSGSGNSYSLTISS | |
| | MEAEDVATYYCFQGSGYPFTFGSGTKLEIK | |
| 11E2 variable | DIQMTQSPSSLSASLGGKVTITCKASQDINKYIAWYQH | 32 |
| light chain GR2 | KPGKGPRLLIHYTSTLQPGIPSRFSGSGSGRDYSFSISNL | |
| | EPEDIATYYCLQYDNLLRTFGGGTKLEIK | |
| 11E2 variable | EVQLQQSGPELEKPGASVKISCKASGYSFTGYNMNWV | 33 |
| heavy chain | KQNNGKSLEWIGNIDPYYGTPNYNQKFKGKATLTVDK | |
| | SSSTAYMQLKSLTSEDSAVYYCALNSLLRLNAMDYWG | |
| | QGTSVTVSS | |
| 16C11 variable | EVQLQQSGPELGKPGASVKISCKASGYSFTGYNMYWV | 34 |
| heavy chain | KQSHRKSLEWIGYIDPYNGDTSYNQKSKGKATLTADR | |
| | SSSTAYMHLNSLTSEDSGIYYCARGAYGSSYAYWGQG | |
| | TLVAVSA | |
| Amino acids 421 | LTQGEDQYYLRVTTVASHTSDSDVPSGVTEVVVKLFD | 41 |
| and 443 of a C- | SDPITVTVPVEVSRKNPKFMETV | |
| terminal portion | AEKALQEYRKKHREE | |
| of a ß-subunit of | | |
| human clusterin | | |

1.-18. (canceled)

19. A method for allowing infiltration of immune cells in a tumor microenvironment, the method comprising administering to a subject in need thereof an anti-clusterin antibody or an antigen binding fragment thereof comprising a light chain variable region comprising a light chain complementarity determining region (CDRL) 1 having the amino acid sequence set forth in SEQ ID NO:1, a CDRL2 having the amino acid sequence set forth in SEQ ID NO:2, and a CDRL3 having the amino acid sequence set forth in SEQ ID NO:3, and a heavy chain variable region comprising a heavy chain complementarity determining region (CDRH) 1 having the amino acid sequence set forth in SEQ ID NO:4, a CDRH2 having the amino acid sequence set forth in SEQ ID NO:5, and a CDRH3 having the amino acid sequence set forth in SEQ ID NO:6, wherein the anti-clusterin antibody or an antigen binding fragment thereof is administered alone or in combination with radiation therapy or with chemotherapy.

20. The method of claim 19, wherein the chemotherapy is selected from an alkylating agent, an anti-metabolite, an alkaloid, an anti-tumor antibiotic or combination thereof or a chemotherapeutic that induces immunogenic modulation.

21. The method of claim 19, wherein the method is used prior to and/or after tumor resection or surgery.

22. The method of claim 19, wherein the method is used as a neoadjuvant therapy.

23. The method of claim 19, wherein the method is used as an adjuvant therapy.

24. The method of claim 19, wherein the subject has a functional immune system, an adequate organ and immune function, is not immunosuppressed and/or has not received immunosuppressive medication within 14, 13, 12, 11, 10, 9, 8, 7, 6, 5, 4, 3, 2 days or 1 day prior to treatment.

25. The method of claim 19, wherein the method is used as a first-line therapy.

26.-28. (canceled)

29. The method of claim 19, wherein the anti-clusterin antibody or antigen binding fragment thereof comprises:
a) a light chain variable region having an amino acid sequence having at least 80% at least 90% identical to the amino acid sequence set forth in SEQ ID NO:9 and a heavy chain variable region having an amino acid sequence at least 90% identical to the amino acid sequence set forth in SEQ ID NO: 10; or
b) a light chain having an amino acid sequence at least 90% identical to the amino acid sequence set forth in SEQ ID NO:11 and a heavy chain having an amino acid sequence at least 90% identical to the amino acid sequence set forth in SEQ ID NO:12.

30.-31. (canceled)

32. The method of claim 19, wherein the immune cells comprise plasmocytes, T cells, CD4⁺ T cells, CD8⁺ T cells, B cells, NK cells and/or macrophages.

33.-38. (canceled)

39. The method of claim 19, wherein the anti-clusterin antibody or antigen binding fragment thereof is administered at a dose of between approximately 3 mg/kg to approximately 20 mg/kg and wherein the chemotherapy comprises docetaxel and is administered at a dose of between approximately 60 mg/m² to approximately 100 mg/m².

40.-46. (canceled)

47. The method of claim 21, wherein subject has a resectable tumor.

48.-49. (canceled)

50. The method of claim 19, wherein the subject in need has or is selected for having:
a) a tumor or one or more lesions characterized as immunologically cold;
b) a tumor or one or more lesions characterized as immunologically warm or hot that is non-responsive to immunotherapy;
c) a carcinoma that has failed prior anti-cancer treatment;
d) a carcinoma that progressed after a first line immune checkpoint therapy;
e) a carcinoma that has failed prior treatment with an immune checkpoint therapy and a platinum-containing doublet treatment; or
f) a carcinoma that has failed prior treatment with an anti-PD1 or PDL-1 immune checkpoint antibody and a platinum-containing doublet treatment.

51.-55. (canceled)

56. The method of claim 19, wherein the subject in need has an early-stage cancer, a late-stage cancer, a metastatic cancer, a carcinoma, a metastatic carcinoma, an endometrial cancer, a breast cancer, a liver cancer, a prostate cancer, a renal cancer, bladder cancer, cervical cancer, an ovarian cancer, a colorectal cancer, a pancreatic cancer, a lung cancer, a gastric cancer, a head and neck cancer, a thyroid cancer, a cholangiocarcinoma, a mesothelioma, a melanoma, a metastatic endometrial cancer, a metastatic breast cancer, a metastatic liver cancer, a metastatic prostate cancer, a metastatic renal cancer, a metastatic bladder cancer, a metastatic cervical cancer, a metastatic ovarian cancer, a metastatic colorectal cancer, a metastatic pancreatic cancer, a metastatic lung cancer, a metastatic gastric cancer, a metastatic head and neck cancer, a metastatic thyroid cancer, a metastatic cholangiocarcinoma, a metastatic mesothelioma, or a metastatic melanoma.

57.-66. (canceled)

67. The method of claim 19, wherein the treatment comprises administering immunotherapy after one or more cycle of the anti-clusterin antibody or antigen binding fragment thereof or of combination therapy.

68. The method of claim 67, wherein the wherein the immunotherapy comprises cellular immunotherapy or an immune checkpoint inhibitor.

69.-86. (canceled)

87. The method of claim 19, wherein the chemotherapy is selected from altretamine, busulfan, carboplatin, carmustine, cisplatin, cyclophosphamide, dacarbazine, ifosfamide, lomustine, melphalan, temozolomide, trabectedin, 5-fluorouracil, 6-mercaptopurine, azacytidine, capecitabine, clofarabine, cytarabine, floxuridine, fludarabine, gemcitabine, methotrexate, pemetrexed, pentostatin, pralatrexate, trifluridine, tipiracil, vincristine, vinblastine, vinorelbine, taxanes, etoposide, teniposide, irinotecan, topotecan, daunorubicin, doxorubicin, doxorubicin liposomal, epirubicin, idarubicin, or valrubicin and optionally wherein the taxane is selected from paclitaxel, docetaxel, Abraxane, Cabazitaxel, larotaxel, milataxel, ortataxel, or tesetaxel.

88. A combination therapy comprising a pharmaceutical composition comprising an anti-clusterin antibody or antigen binding fragment thereof formulated for administration at a dose of between approximately 3 mg/kg to approximately 20 mg/kg and a pharmaceutical composition comprising docetaxel formulated for administration at a dose of approximately 60 mg/m² to 100 mg/m², wherein the anti-clusterin antibody or antigen binding fragment thereof comprises a light chain variable region comprising a light chain complementarity determining region (CDRL) 1 having the amino acid sequence set forth in SEQ ID NO:1, a CDRL2 having the amino acid sequence set forth in SEQ ID NO:2, and a CDRL3 having the amino acid sequence set forth in SEQ ID NO:3, and a heavy chain variable region comprising a heavy chain complementarity determining region (CDRH) 1 having the amino acid sequence set forth in SEQ ID NO:4, a CDRH2 having the amino acid sequence set forth in SEQ ID NO:5, and a CDRH3 having the amino acid sequence set forth in SEQ ID NO:6.

89. The combination therapy of claim 88, wherein the anti-clusterin antibody or antigen binding fragment thereof comprises:
a) a light chain variable region having an amino acid sequence at least 90% identical to the amino acid sequence set forth in SEQ ID NO:9 and a heavy chain variable region having an amino acid sequence at least 90% identical to the amino acid sequence set forth in SEQ ID NO: 10; or
b) a light chain having an amino acid sequence at least 90% identical to the amino acid sequence set forth in SEQ ID NO:11 and a heavy chain having an amino acid sequence at least 80% identical to the amino acid sequence set forth in SEQ ID NO:12.

90. A method for treating a subject in need having cancer, the method comprising administering a combination therapy comprising a pharmaceutical composition comprising an anti-clusterin antibody or antigen binding fragment thereof formulated for administration at a dose of between approximately 3 mg/kg to approximately 20 mg/kg and a pharmaceutical composition comprising docetaxel formulated for administration at a dose of approximately 60 mg/m² to 100 mg/m², wherein the anti-clusterin antibody or antigen binding fragment thereof comprises a light chain variable region comprising a light chain complementarity determining region (CDRL) 1 having the amino acid sequence set forth in SEQ ID NO:1, a CDRL2 having the amino acid sequence set forth in SEQ ID NO:2, and a CDRL3 having the amino acid sequence set forth in SEQ ID NO:3, and a heavy chain variable region comprising a heavy chain complementarity determining region (CDRH) 1 having the amino acid sequence set forth in SEQ ID NO:4, a CDRH2 having the amino acid sequence set forth in SEQ ID NO:5, and a CDRH3 having the amino acid sequence set forth in SEQ ID NO:6.

91. The method of claim 90, wherein the subject in need has an early-stage cancer, a late-stage cancer, a metastatic cancer, a carcinoma, a metastatic carcinoma, an endometrial cancer, a breast cancer, a liver cancer, a prostate cancer, a renal cancer, bladder cancer, cervical cancer, an ovarian cancer, a colorectal cancer, a pancreatic cancer, a lung cancer, a gastric cancer, a head and neck cancer, a thyroid cancer, a cholangiocarcinoma, a mesothelioma, a melanoma, a metastatic endometrial cancer, a metastatic breast cancer, a metastatic liver cancer, a metastatic prostate cancer, a metastatic renal cancer, a metastatic bladder cancer, a metastatic cervical cancer, a metastatic ovarian cancer, a metastatic colorectal cancer, a metastatic pancreatic cancer, a metastatic lung cancer, a metastatic gastric cancer, a metastatic head and neck cancer, a metastatic thyroid cancer, a metastatic cholangiocarcinoma, a metastatic mesothelioma, or a metastatic melanoma.

92. The method of claim 90, wherein the subject in need:
a) has a functional immune system,
b) has an adequate organ and immune function,
c) is not immunosuppressed,
d) has not received an immunosuppressive medication within 14, 13, 12, 11, 10, 9, 8, 7, 6, 5, 4, 3, 2 days or 1 day prior to treatment and/or
e) has or is selected for having:
i) a tumor or one or more lesions characterized as immunologically cold;
ii) a tumor or one or more lesions characterized as immunologically warm or hot that is non-responsive to immunotherapy;
iii) a carcinoma that has failed prior anti-cancer treatment;
iv) a carcinoma that progressed after a first line immune checkpoint therapy;
v) a carcinoma that has failed prior treatment with an immune checkpoint therapy and a platinum-containing doublet treatment; or
vi) a carcinoma that has failed prior treatment with an anti-PD1 or PDL-1 immune checkpoint antibody and a platinum-containing doublet treatment.

93. The method of claim 90, wherein the anti-clusterin antibody or antigen binding fragment thereof is administered at a dose of approximately 6 mg/kg, at a dose of approximately 9 mg/kg, or at a dose of approximately 12 mg/kg and/or wherein docetaxel is administered at a dose of approximately 60 mg/m2 or at a dose of approximately 75 mg/m².

94. The method of claim 90, wherein:
a) the anti-clusterin antibody or antigen binding fragment thereof is administered at a dose of approximately 12 mg/kg once weekly and docetaxel is administered at a dose of approximately 75 mg/m² once every three weeks;
b) the anti-clusterin antibody or antigen binding fragment thereof is administered at a dose of approximately 12 mg/kg once weekly and docetaxel is administered at a dose of approximately 60 mg/m² once every three weeks;
c) the anti-clusterin antibody or antigen binding fragment thereof is administered at a dose of approximately 9 mg/kg once weekly and docetaxel is administered at a dose of approximately 75 mg/m² once every three weeks;
d) the anti-clusterin antibody or antigen binding fragment thereof is administered at a dose of approximately 9 mg/kg once weekly and docetaxel is administered at a dose of approximately 60 mg/m² once every three weeks;
e) the anti-clusterin antibody or antigen binding fragment thereof is administered at a dose of approximately 6 mg/kg once weekly and docetaxel is administered at a dose of approximately 75 mg/m² once every three weeks; or
f) the anti-clusterin antibody or antigen binding fragment thereof is administered at a dose of approximately 6 mg/kg once weekly and docetaxel is administered at a dose of approximately 60 mg/m² once every three weeks.

95. A method for allowing infiltration of immune cells in a tumor microenvironment, the method comprising administering to a subject in need thereof, an anti-clusterin antibody or an antigen binding fragment thereof that competes with an antibody comprising a light chain variable region having an amino acid sequence set forth in SEQ ID NO:9 and a heavy chain variable region having an amino acid sequence set forth in SEQ ID NO:10 for the binding of clusterin, wherein the anti-clusterin antibody or antigen binding fragment thereof is administered alone or in combination with docetaxel.
